(19) United States
(12) Patent Application Publication
(10) Pub. No.: US 2009/0136449 A1
(43) Pub. Date: May 28, 2009

(54) Tetracyclic Indole Derivatives as Antiviral Agents

(76) Inventors: Marcello Di Filippo, Rome (IT); Steven Harper, Albano Laziale (Rome) (IT); Frank Narjes, Ariccia (IT); Barbara Pacini, Pomezia (IT); Alessia Petrocchi, Rome (IT); Michael Rowley, Axa (IT); Ian Stansfield, Rome (IT)

Correspondence Address: MERCK AND CO., INC, P O BOX 2000, RAHWAY, NJ, 07065-0907, US

(21) Appl. No.: 12/084,846

(22) PCT Filed: Nov. 9, 2006
(86) PCT No.: PCT/GB2006/050378

§ 371 Date: May 9, 2008

(30) Foreign Application Priority Data
Nov. 10, 2005 (GB) 0522881.2

(51) Int. Cl. A61K 38/21; C07D 487/04; A61K 31/407; C07D 498/04

(52) U.S. Cl. 424/85.5; 540/471; 514/410; 540/468; 424/85.7; 424/85.6

(57) ABSTRACT

The present invention relates to tetracyclic indole derivatives of formula (I); wherein Ar, D¹, D², D³, D⁴, W, X, Y and Z are defined herein, and pharmaceutically acceptable salts thereof, pharmaceutical compositions comprising them, and their use for the treatment or prevention of infection by hepatitis C virus.

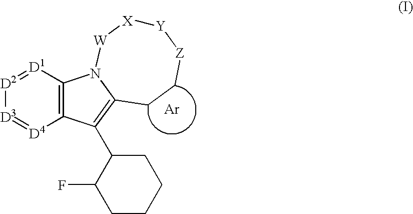

The present invention relates to fluorinated tetracyclic indole compounds, to pharmaceutical compositions containing them, to their use in the prevention and treatment of hepatitis C infections and to methods of preparation of such compounds and compositions.

Hepatitis C (HCV) is a cause of viral infections. There is as yet no adequate treatment for HCV infection but it is believed that inhibition of its RNA polymerase in mammals, particularly humans, would be of benefit.

Published International patent application WO 93/00334 (Fidia-Georgetown Institute for the Neurosciences) discloses the following indole derivatives:

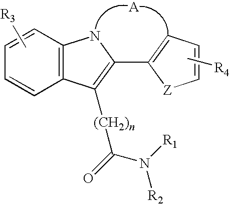

where A, Z, R₁, R₂, R₃, R₄ and n are defined therein, as useful in compositions and methods for treating psychiatric and neurological disorders. However, this document does not disclose the use of tetracyclic indole derivatives in treating or preventing viral infections.

Published International patent application WO 2005/080399 (Japan Tobacco Inc.) discloses the following fused heterotetracyclic compounds:

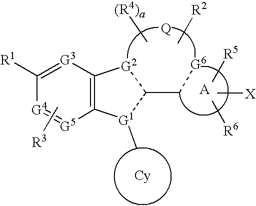

where A, X, Cy, G¹, G², G³, G⁴, G⁵, G⁶, R¹, R², R³, R⁴, R⁵, R⁶ and a are defined therein, and their use as HCV polymerase inhibitors.

Published International patent application WO 2006/020082 (Bristol-Myers Squibb Company) discloses the following fused tetracyclic compounds:

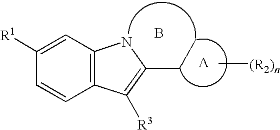

where A, B, R¹, R², R³ and n are defined therein, and their use in treating hepatitis C.

Published International applications WO2006/046030 and WO2006/046039 (both Istituto Di Ricerche Di Biologia Molecolare P. Angeletti SpA) disclose certain tetracyclic indole derivatives:

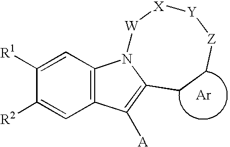

wherein R¹, R², A, Ar, W, X, Y, and Z are defined therein, useful for the treatment or prevention of infection by hepatitis C virus.

Thus, the present invention provides the compound of the formula (I):

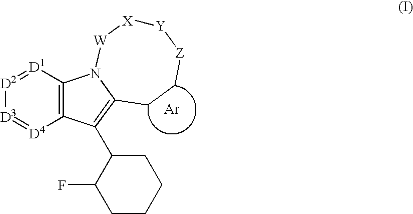

wherein

Ar is a moiety containing at least one aromatic ring and possesses 5-, 6-, 9- or 10-ring atoms optionally containing 1, 2 or 3 heteroatoms independently selected from N, O and S, which ring is optionally substituted at any substitutable position by groups Q¹ and Q²;

Q¹ is halogen, hydroxy, C₁₋₆alkyl, C₁₋₆alkoxy, aryl, heteroaryl, CONR^{c}R^{d}, (CH₂)₀₋₃NR^{c}R^{d}, O(CH₂)₀₋₃C₃₋₈cycloalkyl, O(CH₂)₁₋₃NR^{c}R^{d}, O(CH₂)₀₋₃CONR^{c}R^{d}, O(CH₂)₀₋₃aryl, OCH(CH₃)aryl, O(CH₂)₀₋₃heteroaryl, OCH(CH₃)heteroaryl or OCHR^{e}R^{f};

R^{c} and R^{d} are each independently selected from hydrogen, C₁₋₄alkyl and C(O)C₁₋₄alkyl;

or R^{c}, R^{d} and the nitrogen atom to which they are attached form a heteroaliphatic ring of 4 to 7 ring atoms, where said ring is optionally substituted by halogen, hydroxy, C₁₋₄alkyl or C₁₋₄alkoxy;

R^{e} and R^{f} are each independently selected from hydrogen and C₁₋₄alkoxy;

or R^{e} and R^{f} are linked by a heteroatom selected from N, O and S to form a heteroaliphatic ring of 4 to 7 ring atoms, where said ring is optionally substituted by halogen, hydroxy, C₁₋₄alkyl or C₁₋₄alkoxy;

and wherein said C₁₋₄alkyl, C₁₋₄alkoxy and aryl groups are optionally substituted by halogen or hydroxy;

Q² is halogen, hydroxy, C₁₋₄alkyl or C₁₋₄alkoxy, where said C₁₋₄alkyl and C₁₋₄alkoxy groups are optionally substituted by halogen or hydroxy;

or Q¹ and Q² may be linked by a bond or a heteroatom selected from N, O and S to form a ring of 4 to 7 atoms, where said ring is optionally substituted by halogen, hydroxy, C₁₋₄alkyl or C₁₋₄alkoxy;

D¹ is N or CR^{a};

D² is N or CR¹;

D³ is N or CR²;

D⁴ is N or CR^{b};

with the proviso that D² and D³ are not both N;

R^{a} and R^{b} are each independently selected from hydrogen, fluorine, chlorine, C₁₋₄alkyl, C₂₋₄alkenyl or C₁₋₄alkoxy, where said C₁₋₄alkyl, C₂₋₄alkenyl and C₁₋₄alkoxy groups are optionally substituted by hydroxy or fluorine;

one of R¹ or R² is hydrogen, halogen, C₁₋₄alkyl, C₁₋₄alkoxy, CN, CO₂H, CO₂C₁₋₄alkyl, aryl, heteroaryl or C(O)NR³R⁴, where said C₁₋₄alkyl, C₁₋₄alkoxy, aryl and heteroaryl groups are optionally substituted by hydroxy or fluorine;

R³ is hydrogen or C₁₋₄alkyl;

R⁴ is hydrogen, C₁₋₄alkyl, C₂₋₄alkenyl, (CH₂)₀₋₃R⁵, SO₂R⁶ or -L-CO₂R²⁰;

R⁵ is NR^{h}Rⁱ, OR^{h}, aryl, heteroaryl or Het;

R^{h} and Rⁱ are each independently selected from hydrogen and C₁₋₄alkyl;

Het is a heteroaliphatic ring of 4 to 7 ring atoms, which ring may contain 1, 2 or 3 heteroatoms selected from N, O or S or a group S(O), S(O)₂, NH or NC₁₋₄alkyl;

R⁶ is C₁₋₄alkyl, C₁₋₄alkenyl or (CH₂)₀₋₃R⁷;

R⁷ is aryl, heteroaryl, C₁₋₄alkyl, C₃₋₈cycloalkyl, CO₂R⁸, Het or NR^{m}Rⁿ, wherein Het is as hereinbefore defined, R^{m} and Rⁿ are each independently selected from hydrogen, C₁₋₄alkyl and CO₂(CH₂)₀₋₃aryl, and R⁸ is hydrogen or C₁₋₆alkyl,

and wherein R⁷ is optionally substituted by halogen, C₁₋₄alkyl or NR^{o}R^{p}, wherein R^{o} and R^{p} are each independently selected from hydrogen and C₁₋₄alkyl;
and where R⁴ is optionally substituted by hydroxy, fluorine, chlorine, C₁₋₄alkyl, ═O, CO₂H or CO₂C₁₋₄alkyl;

or R³, R⁴ and the nitrogen atom to which they are attached form a heteroaliphatic ring of 4 to 7 ring atoms, where said ring is optionally substituted by halogen, hydroxy, ═O, C₁₋₄alkyl or C₁₋₄alkoxy;

the other of R¹ and R² is hydrogen, fluorine, chlorine, C₁₋₄alkyl, C₂₋₄alkenyl or C₁₋₄alkoxy, where said C₁₋₄alkyl, C₂₋₄alkenyl and C₁₋₄alkoxy groups are optionally substituted by hydroxy or fluorine;

R²⁰ is hydrogen or C₁₋₆alkyl;

L is

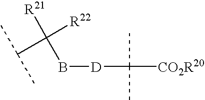

wherein R²¹ and R²² are independently selected from hydrogen, halogen, C₁₋₄alkyl, C₂₋₄alkenyl or C₁₋₄alkoxy;

or R²¹ and R²² are linked to form a C₃₋₈cycloalkyl group;

B is aryl, heteroaryl or CONR²³R²⁴, optionally substituted by halogen, C₁₋₄alkyl, C₂₋₄alkenyl or C₁₋₄alkoxy;

R²³ is hydrogen or C₁₋₆alkyl;

or R²³ is linked to R²¹ and/or R²² to form a 5- to 10-membered ring, where said ring may be saturated, partially saturated or unsaturated, and where said ring is optionally substituted by halogen, C₁₋₄alkyl, C₂₋₄alkenyl, C₂₋₄alkynyl or C₁₋₄alkoxy;

R²⁴ is aryl or heteroaryl;

or R²³, R²⁴ and the nitrogen atom to which they are attached form a 5- to 10-membered mono- or bi-cyclic ring system, where said ring may be saturated, partially saturated or unsaturated, and where said ring is optionally substituted by halogen, C₁₋₄alkyl, C₂₋₄alkenyl, C₂₋₄alkynyl or C₁₋₄alkoxy;

D is a bond, C₁₋₆alkylene, C₂₋₆alkenylene, C₂₋₆alkynylene, aryl or heteroaryl, where said aryl or heteroaryl is optionally substituted by halogen, C₁₋₄alkyl or C₂₋₄alkenyl;

W and Z are independently selected from a bond, C═O, O, S, S(O), S(O)₂, —(CR¹⁰R¹¹)—(CR¹²R¹³)₀₋₁— and NR¹⁰;

X and Y are independently selected from a bond, C═O, O, —CR¹⁴R¹⁵—, —CR¹⁴(OR¹⁵)— and NR¹⁴;

and none, one or two of W, X, Y and Z are a bond;

R¹⁰, R¹¹, R¹², R¹³, R¹⁴ and R¹⁵ are each independently selected from hydrogen, hydroxy, C₁₋₆alkyl, C₂₋₆alkenyl, C₁₋₆alkoxy, C(O)C₁₋₆alkyl, (CH₂)₀₋₃C₃₋₈cycloalkyl, (CH₂)₀₋₃heteroaryl, (CH₂)₀₋₃Het, (CH₂)₀₋₃C(O)(CH₂)₀₋₃Het, (CH₂)₀₋₃NR¹⁶R¹⁷, (CH₂)₀₋₃C(O)(CH₂)₀₋₃NR¹⁶R¹⁷ and NHC(O)(CH₂)₀₋₃NR¹⁶R¹⁷;

R¹⁶ and R¹⁷ are independently selected from hydrogen, C₁₋₆alkyl and (CH₂)₀₋₄NR¹⁸R¹⁹; or R¹⁶, R¹⁷ and the nitrogen atom to which they are attached form a heteroaliphatic ring of 4 to 7 ring atoms, which ring may optionally contain 1 or 2 more heteroatoms selected from O or S or a group S(O), S(O)₂, NH or NC₁₋₄alkyl, and which ring is optionally substituted by halogen, hydroxy, C₁₋₄alkyl or C₁₋₄alkoxy;

R¹⁸ and R¹⁹ are independently selected from hydrogen and C₁₋₆alkyl;

or R¹⁸, R¹⁹ and the nitrogen atom to which they are attached form a heteroaliphatic ring of 4 to 7 ring atoms, which ring may optionally contain 1 or 2 more heteroatoms selected from O or S or a group S(O), S(O)₂, NH or NC₁₋₄alkyl, and which ring is optionally substituted by halogen, hydroxy, C₁₋₄alkyl or C₁₋₄alkoxy;
and pharmaceutically acceptable salts thereof.

In one embodiment of the present invention, there is provided the compound of the formula (Io):

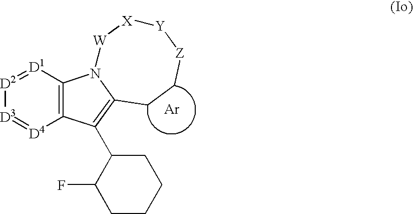

wherein

Ar is a moiety containing at least one aromatic ring and possesses 5-, 6-, 9- or 10-ring atoms optionally containing 1, 2 or 3 heteroatoms independently selected from N, O and S, which ring is optionally substituted at any substitutable position by groups Q¹ and Q²;

Q¹ is halogen, hydroxy, C₁₋₆alkyl, C₁₋₆alkoxy, aryl, heteroaryl, CONR^{c}R^{d}, (CH₂)₀₋₃NR^{c}R^{d}, O(CH₂)₀₋₃C₃₋₈cycloalkyl, O(CH₂)₁₋₃NR^{c}R^{d}, O(CH₂)₀₋₃CONR^{c}R^{d}, O(CH₂)₀₋₃aryl, O(CH₂)₀₋₃heteroaryl, OCHR^{e}R^{f};

R^{c} and R^{d} are each independently selected from hydrogen, C₁₋₄alkyl and C(O)C₁₋₄alkyl;

or R^{c}, R^{d} and the nitrogen atom to which they are attached form a heteroaliphatic ring of 4 to 7 ring atoms, where said ring is optionally substituted by halogen, hydroxy, C₁₋₄alkyl or C₁₋₄alkoxy;

R^{e} and R^{f} are each independently selected from hydrogen and C₁₋₄alkoxy;

or R^{e} and R^{f} are linked by a heteroatom selected from N, O and S to form a heteroaliphatic ring of 4 to 7 ring atoms, where said ring is optionally substituted by halogen, hydroxy, C₁₋₄alkyl or C₁₋₄alkoxy;

and wherein said C₁₋₄alkyl, C₁₋₄alkoxy and aryl groups are optionally substituted by halogen or hydroxy;

Q² is halogen, hydroxy, C₁₋₄alkyl or C₁₋₄alkoxy, where said C₁₋₄alkyl and C₁₋₄alkoxy groups are optionally substituted by halogen or hydroxy;

or Q¹ and Q² may be linked by a bond or a heteroatom selected from N, O and S to form a ring of 4 to 7 atoms, where said ring is optionally substituted by halogen, hydroxy, C₁₋₄alkyl or C₁₋₄alkoxy;

D¹ is N or CR^{a};

D² is N or CR¹;

D³ is N or CR²;

D⁴ is N or CR^{b};

with the proviso that D² and D³ are not both N;

R^{a} and R^{b} are each independently selected from hydrogen, fluorine, chlorine, C₁₋₄alkyl, C₂₋₄alkenyl or C₁₋₄alkoxy, where said C₁₋₄alkyl, C₂₋₄alkenyl and C₁₋₄alkoxy groups are optionally substituted by hydroxy or fluorine;

one of R¹ or R² is hydrogen, halogen, C₁₋₄alkyl, C₁₋₄alkoxy, CN, CO₂H, CO₂C₁₋₄alkyl, aryl, heteroaryl or C(O)NR³R⁴, where said C₁₋₄alkyl, C₁₋₄alkoxy, aryl and heteroaryl groups are optionally substituted by hydroxy or fluorine;

R³ is hydrogen or C₁₋₄alkyl;

R⁴ is hydrogen, C₁₋₄alkyl, C₂₋₄alkenyl, (CH₂)₀₋₃R⁵ or SO₂R⁶;

R⁵ and R⁶ are as defined in relation to formula (I);

and where R⁴ is optionally substituted by hydroxy, fluorine, chlorine, C₁₋₄alkyl, ═O, CO₂H or CO₂C₁₋₄alkyl;

or R³, R⁴ and the nitrogen atom to which they are attached form a heteroaliphatic ring of 4 to 7 ring atoms, where said ring is optionally substituted by halogen, hydroxy, ═O, C₁₋₄alkyl or C₁₋₄alkoxy;

the other of R¹ and R² is hydrogen, fluorine, chlorine, C₁₋₄alkyl, C₂₋₄alkenyl or C₁₋₄alkoxy, where said C₁₋₄alkyl, C₂₋₄alkenyl and C₁₋₄alkoxy groups are optionally substituted by hydroxy or fluorine;

W, X, Y and Z are as defined in relation to formula (I);

and pharmaceutically acceptable salts thereof.

In another embodiment of the present invention, Ar is a five- or six-membered aromatic ring optionally containing 1, 2 or 3 heteroatoms independently selected from N, O and S, and which ring is optionally substituted by groups Q¹ and Q² as hereinbefore defined.

Preferably, Ar is a five- or six-membered aromatic ring optionally containing a heteroatom selected from N, O or S such as phenyl, pyridyl, pyrrolyl, furanyl and thienyl, which ring is optionally substituted by groups Q¹ and Q² as hereinbefore defined. More preferably, Ar is phenyl or 2-thienyl, particularly phenyl, optionally substituted by groups Q¹ and Q² as hereinbefore defined.

Preferably, Q¹ is halogen, hydroxy, C₁₋₆alkyl or C₁₋₆alkoxy, O(CH₂)₀₋₃C₃₋₈cycloalkyl, O(CH₂)₁₋₃NR^{c}R^{d}, O(CH₂)₀₋₃aryl, OCH(CH₃)aryl, O(CH₂)₀₋₃heteroaryl or OCH(CH₃)heteroaryl, where R^{c} and R^{d} are as hereinbefore defined. More preferably, Q¹ is halogen, C₁₋₄alkyl, C₁₋₄alkoxy, O(CH₂)₀₋₃C₃₋₆cycloalkyl, O(CH₂)₁₋₂NR^{c}R^{d}, O(CH₂)₀₋₃phenyl, OCH(CH₃)phenyl, O(CH₂)₀₋₂heteroaryl or OCH(CH₃)heteroaryl, where R^{c} and R^{d} are independently selected from hydrogen and C₁₋₄alkyl, and heteroaryl is a 5- or 6-membered heteroaromatic ring containing 1, 2 or 3 heteroatoms selected from N, O and S. Most preferably, Q¹ is halogen, C₁₋₂alkyl, C₁₋₃alkoxy, O(CH₂)C₃₋₆cycloalkyl, O(CH₂)₁₋₂N(C₁₋₄alkyl)₂, O(CH₂)₀₋₁phenyl, OCH(CH₃)phenyl, O(CH₂)₀₋₁heteroaryl or OCH(CH₃)heteroaryl, where heteroaryl is a 5- or 6-membered heteroaromatic ring containing 1 or 2 heteroatoms selected from N and S.

Examples of suitable Q¹ groups include fluorine, chlorine, methyl, methoxy, ethoxy, n-propoxy,

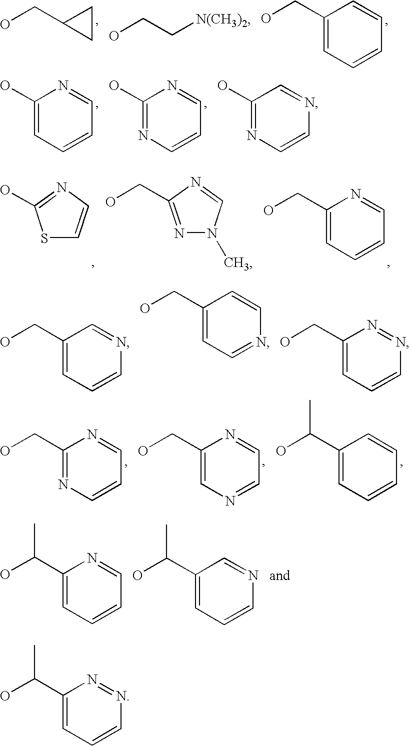

Preferably Q² is absent.

In a further embodiment, D¹ is CR^{a} wherein R^{a} is as hereinbefore defined. Preferably, R^{a} is hydrogen, fluorine, methyl or trifluoromethyl. More preferably, R^{a} is hydrogen.

In a further embodiment, D⁴ is CR^{b} wherein R^{b} is as hereinbefore defined. Preferably, R^{b} is hydrogen, fluorine, methyl, methoxy or trifluoromethyl. More preferably, R^{b} is hydrogen.

In a further embodiment, D² is CR¹ wherein R¹ is as hereinbefore defined. Preferably, R¹ is CO₂H, CO₂C₁₋₄alkyl, heteroaryl or C(O)NR³R⁴, where R³ and R⁴ are as hereinbefore defined. More preferably, R¹ is CO₂H, heteroaryl or C(O)NHR⁴, where heteroaryl is a 5- or 6-membered heteroaromatic ring containing 1 to 4 nitrogen atoms, and R⁴ is as hereinbefore defined. Most preferably, R¹ is CO₂H, a 5-membered heteroaromatic ring containing 1 to 4 nitrogen atoms, C(O)NH(C₁₋₄alkyl), C(O)NHSO₂R⁶ or C(O)NH-L-CO₂R²⁰, where R⁶, L and R²⁰ are as hereinbefore defined. Examples of suitable R¹ groups include CO₂H, tetrazolyl, C(O)NH(CH₃), C(O)NHSO₂(CH₂CH₃), C(O)NHSO₂N(CH₃)₂ and

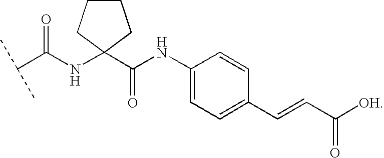

In a further embodiment, D³ is CR² wherein R² is as hereinbefore defined. Preferably, R² is hydrogen, fluorine, chlorine, C₁₋₄alkyl, C₂₋₄alkenyl or C₁₋₄alkoxy, where said C₁₋₄alkyl, C₂₋₄alkenyl and C₁₋₄alkoxy groups are optionally substituted by hydroxy or fluorine. More preferably, R² is hydrogen or C₁₋₄alkyl. Most preferably, R² is hydrogen.

In a further embodiment, W is a bond, C═O, —(CR¹⁰R¹¹)—(CR¹²R¹³)₀₋₁— or NR¹⁰ where R¹⁰, R¹¹, R¹² and R¹³ are as hereinbefore defined. Preferably, W is —(CR¹⁰R¹¹)—(CR¹²R¹³)₀₋₁— such as —CH₂—, —CH₂CH₂—, —CH(CH₃)—, —CH(CH₃)—CH(CH₃)—, —C(CH₃)₂— or —C(CH₃)₂—C(CH₃)₂—. More preferably, W is —CH₂— or —CH₂CH₂—. Most preferably, W is —CH₂—.

In a further embodiment, Z is a bond, C═O, O, —(CR¹⁰R¹¹)—(CR¹²R¹³)₀₋₁— or NR¹⁰ where R¹⁰, R¹¹, R¹² and R¹³ are as hereinbefore defined. Preferably, Z is a bond, O or —(CR¹⁰R¹¹)—(CR¹²R¹³)₀₋₁—. More preferably, Z is a bond O or —CH₂—.

In a further embodiment, X is C═O, —CR¹⁴R¹⁵— or —CR¹⁴(OR¹⁵)— where R¹⁴ and R¹⁵ are as hereinbefore defined.

Preferably, X is CO, —CHR¹⁵— or —CH(OR¹⁵)— where R¹⁵ are as hereinbefore defined. More preferably, X is C═O, —CH₂—, —CH[(CH₂)₀₋₃NR¹⁶NR¹⁷]— or CH(O[(CH₂)₁₋₃NR¹⁶R¹⁷])—, where R¹⁶ and R¹⁷ are as hereinbefore defined. Most preferably, X is C═O, —CH₂—, —CH[NR¹⁶R¹⁷]—, CH₂CH₂[NR¹⁶R¹⁷]— or —CH(O[(CH₂)₁₋₃N(C₁₋₄alkyl)₂])—. Especially, X is C═O, —CH₂—, —CH[N(CH₃)₂]—, —CH[N(CH₃)CH₂CH₂N(CH₃)₂]—, CH₂CH₂N(CH₃)₂, CH₂CH₂N(C₂H₅)₂ or —CH(OCH₂CH₂N(CH₃)₂).

In a further embodiment, Y is —CR¹⁴R¹⁵— or NR¹⁴ where R¹⁴ and R¹⁵ are as hereinbefore defined. Preferably, Y is —CH₂— or R¹⁴ where R¹⁴ is hydrogen, C₁₋₆alkyl, (CH₂)₀₋₃C₃₋₈cycloalkyl, (CH₂)₀₋₃C(O)(CH₂)₀₋₃Het, (CH₂)₁₋₃NR¹⁶R¹⁷, (CH₂)₀₋₃heteroaryl or (CH₂)₀₋₃C(O)(CH₂)₀₋₃NR¹⁶R¹⁷, where R¹⁶ and R¹⁷ are as hereinbefore defined. More preferably, Y is —CH₂— or NR¹⁴ where R¹⁴ is hydrogen, C₁₋₄alkyl, C₃₋₆cycloalkyl, C(O)Het, (CH₂)₂NR¹⁶R¹⁷, (CH₂)₀₋₃pyridyl or (CH₂)₀₋₁C(O)(CH₂)₀₋₁NR¹⁶R¹⁷, where R¹⁶ and R¹⁷ are independently selected from hydrogen and C₁₋₄alkyl,

or R¹⁶ and R¹⁷, together with the nitrogen atom to which they are attached, form a heteroaliphatic ring of 5or 6 ring atoms, which ring may optionally contain one oxygen atom and/or a NH or N(C₁₋₄alkyl) group.

Examples of suitable R¹⁴ groups include hydrogen, methyl, ethyl, n-propyl, i-propyl, cyclopropyl,

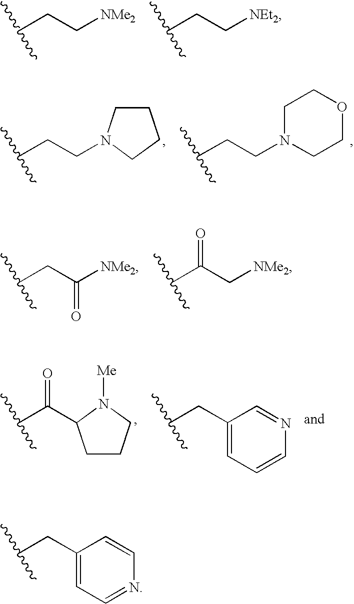

In one embodiment, the compound of formula (I) as hereinbefore described has the following relative stereochemical configuration:

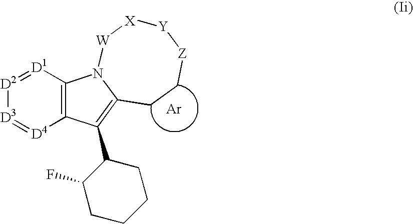

One favoured group of compounds of the present invention is of formula (Ia) and pharmaceutically acceptable salts thereof:

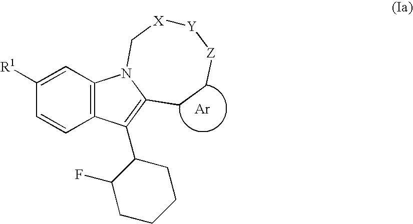

wherein Ar, R¹, X, Y and Z are as defined in relation to formula (Io).

Preferably, the compound of formula (Ia) as hereinbefore described has the following relative stereochemical configuration:

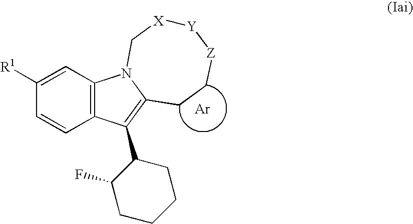

It will be appreciated that the preferred definitions of the various substituents recited herein may be taken alone or in combination and, unless otherwise stated, apply to the generic formula for compounds of the present invention as well as the preferred classes of compound represented by formulae (Io), (Ii), (Ia) and (Iai).

When any variable occurs more than one time in formula (I) or in any substituent, its definition on each occurrence is independent of its definition at every other occurrence.

As used herein, the term “alkyl” or “alkoxy” as a group or part of a group means that the group is straight or branched. Examples of suitable alkyl groups include methyl, ethyl, n-propyl, i-propyl, n-butyl, s-butyl and t-butyl. Examples of suitable alkoxy groups include methoxy, ethoxy, n-propoxy, i-propoxy, n-butoxy, s-butoxy and t-butoxy.

The cycloalkyl groups referred to herein may represent, for example, cyclopropyl, cyclobutyl, cyclopentyl or cyclohexyl. A suitable cycloalkylalkyl group may be, for example, cyclopropylmethyl.

As used herein, the term “alkenyl” as a group or part of a group means that the group is straight or branched. Examples of suitable alkenyl groups include vinyl and allyl.

When used herein, the term “halogen” means fluorine, chlorine, bromine and iodine.

When used herein, the term “aryl” as a group or part of a group means a carbocyclic aromatic ring. Examples of suitable aryl groups include phenyl and naphthyl.

When used herein, the term “heteroaryl” as a group or part of a group means a 5- to 10-membered heteroaromatic ring system containing 1 to 4 heteroatoms selected from N, O and S. Particular examples of such groups include pyrrolyl, furanyl, thienyl, pyridyl, pyrazolyl, imidazolyl, oxazolyl, isoxazolyl, thiazolyl, isothiazolyl, pyrazinyl, pyrimidinyl, pyridazinyl, triazolyl, oxadiazolyl, thiadiazolyl, triazinyl, tetrazolyl, indolyl, benzothienyl, benzimidazolyl and quinolinyl.

When used herein, the term “Het” as a group or part of a group means a heteroaliphatic ring of 4 to 7 atoms, which ring may contain 1, 2 or 3 heteroatoms selected from N, O and S or a group S(O), S(O)₂, NH or NC₁₋₄alkyl.

Where a compound or group is described as “optionally substituted” one or more substituents may be present. Optional substituents may be attached to the compounds or groups which they substitute in a variety of ways, either directly or through a connecting group of which the following are examples: amine, amide, ester, ether, thioether, sulfonamide, sulfamide, sulfoxide, urea, thiourea and urethane. As appropriate an optional substituent may itself be substituted by another substituent, the latter being connected directly to the former or through a connecting group such as those exemplified above.

Specific compounds within the scope of this invention include those named in the Examples and Tables below and their pharmaceutically acceptable salts.

For use in medicine, the salts of the compounds of formula (I) will be non-toxic pharmaceutically acceptable salts. Other salts may, however, be useful in the preparation of the compounds according to the invention or of their non-toxic pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, fumaric acid, p-toluenesulfonic acid, maleic acid, succinic acid, acetic acid, citric acid, tartaric acid, carbonic acid, phosphoric acid or sulfuric acid. Salts of amine groups may also comprise quaternary ammonium salts in which the amino nitrogen atom carries a suitable organic group such as an alkyl, alkenyl, alkynyl or aralkyl moiety. Furthermore, where the compounds of the invention carry an acidic moiety, suitable pharmaceutically acceptable salts thereof may include metal salts such as alkali metal salts, e.g. sodium or potassium salts; and alkaline earth metal salts, e.g. calcium or magnesium salts.

The salts may be formed by conventional means, such as by reacting the free base form of the product with one or more equivalents of the appropriate acid in a solvent or medium in which the salt is insoluble, or in a solvent such as water which is removed in vacuo or by freeze drying or by exchanging the anions of an existing salt for another anion on a suitable ion exchange resin.

The present invention includes within its scope prodrugs of the compounds of formula (I) above. In general, such prodrugs will be functional derivatives of the compounds of formula (I) which are readily convertible in vivo into the required compound of formula (I). Conventional procedures for the selection and preparation of suitable prodrug derivatives are described, for example, in “Design of Prodrugs”, ed. H. Bundgaard, Elsevier, 1985.

A prodrug may be a pharmacologically inactive derivative of a biologically active substance (the “parent drug” or “parent molecule”) that requires transformation within the body in order to release the active drug, and that has improved delivery properties over the parent drug molecule. The transformation in vivo may be, for example, as the result of some metabolic process, such as chemical or enzymatic hydrolysis of a carboxylic, phosphoric or sulfate ester, or reduction or oxidation of a susceptible functionality.

The present invention includes within its scope solvates of the compounds of formula (I) and salts thereof, for example, hydrates.

The present invention also includes within its scope N-oxides of the compounds of formula (I).

The present invention also includes within its scope any enantiomers, diastereomers, geometric isomers and tautomers of the compounds of formula (I). It is to be understood that all such isomers and mixtures thereof are encompassed within the scope of the invention.

The present invention further provides a compound of formula (I) or a pharmaceutically acceptable salt thereof for use in therapy.

In another aspect, the invention provides the use of a compound of formula (I) as defined above, or a pharmaceutically acceptable salt thereof, for the manufacture of a medicament for treatment or prevention of infection by hepatitis C virus in a human or animal.

A further aspect of the invention provides a pharmaceutical composition comprising a compound of formula (I) as defined above, or a pharmaceutically acceptable salt thereof, in association with a pharmaceutically acceptable carrier. The composition may be in any suitable form, depending on the intended method of administration. It may for example be in the form of a tablet, capsule or liquid for oral administration, or of a solution or suspension for administration parenterally.

The pharmaceutical compositions optionally also include one or more other agents for the treatment of viral infections such as an antiviral agent, or an immunomodulatory agent such as α-, β- or γ-interferon.

In a further aspect, the invention provides a method of inhibiting hepatitis C virus polymerase and/or of treating or preventing an illness due to hepatitis C virus, the method involving administering to a human or animal (preferably mammalian) subject suffering from the condition a therapeutically or prophylactically effective amount of the pharmaceutical composition described above or of a compound of formula (I) as defined above, or a pharmaceutically acceptable salt thereof. “Effective amount” means an amount sufficient to cause a benefit to the subject or at least to cause a change in the subject's condition.

The dosage rate at which the compound is administered will depend on a variety of factors including the activity of the specific compound employed, the metabolic stability and length of action of that compound, the age of the patient, body weight, general health, sex, diet, mode and time of administration, rate of excretion, drug combination, the severity of the particular condition and the host undergoing therapy. Suitable dosage levels may be of the order of 0.02 to 5 or 10 g per day, with oral dosages two to five times higher. For instance, administration of from 1 to 20 or 50 mg of the compound per kg of body weight from one to three times per day may be in order. Appropriate values are selectable by routine testing. The compound may be administered alone or in combination with other treatments, either simultaneously or sequentially. For instance, it may be administered in combination with effective amounts of antiviral agents, immunomodulators, anti-infectives or vaccines known to those of ordinary skill in the art. It may be administered by any suitable route, including orally, intravenously, cutaneously and subcutaneously. It may be administered directly to a suitable site or in a manner in which it targets a particular site, such as a certain type of cell. Suitable targeting methods are already known.

An additional aspect of the invention provides a method of preparation of a pharmaceutical composition, involving admixing at least one compound of formula (I) as defined above, or a pharmaceutically acceptable salt thereof, with one or more pharmaceutically acceptable adjuvants, diluents or carriers and/or with one or more other therapeutically or prophylactically active agents.

The present invention also provides a process for the preparation of compounds of formula (I).

According to a general process (a), compounds of formula (I) where Y is NR¹⁴ may be prepared by internal ring closure of a compound of formula (II):

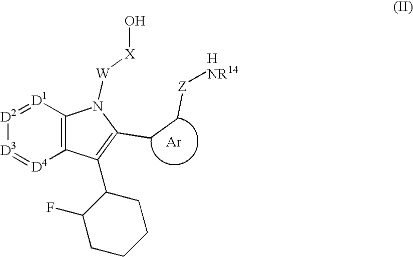

wherein R¹⁴, D¹, D², D³, D⁴, Ar, W, X and Z are defined in relation to formula (I). The reaction is conveniently performed in the presence of a coupling reagent, such as HATU, and a base, such as diisopropylethylamine, in a solvent Suitable solvents include dichloromethane.

According to a general process (b), compounds of formula (I) where Y is NR¹⁴ and Z is —CH₂— may be prepared by reduction and internal ring closure of a compound of formula (III):

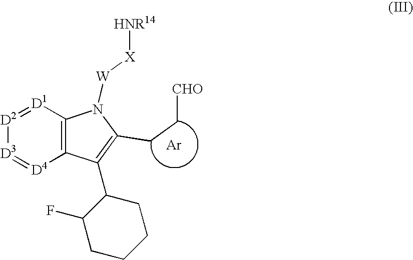

wherein R¹⁴, D¹, D², D³, D⁴, Ar, W and X are as defined in relation to formula (I). The reduction is conveniently performed in the presence of a mild reducing agent, such as sodium cyanoborohydride, in a suitable solvent, such as methanol. The ring closure is conveniently performed in the presence of a coupling reagent, such as HATU, and a base, such as diisopropylethylamine, in a solvent. Suitable solvents include dichloromethane.

According to a general process (c), compounds of formula (I) may be prepared by internal ring closure of a compound of formula (IV):

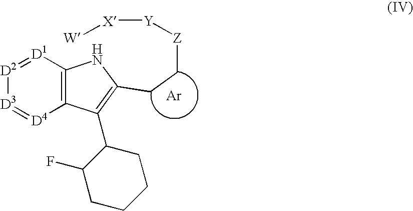

wherein R¹, R², A, Ar, Y and Z are as defined in relation to formula (I) and X′ is X as defined in relation to formula (I) or is converted to X during or after the cyclisation reaction, and W′ is W as defined in relation to formula (I) or is converted to W during or after the cyclisation reaction. W′ and X′ may be suitable activated precursors of groups W and X respectively which can be converted into W and X respectively during the ring closure or after it using methods described in the accompanying Schemes and Examples or known to the person skilled in the art. For example, W′ may be CH₂-halogen or W′ and X′ together may be an epoxide or aziridine group. When W′ is CH₂-halogen, such as CH₂—Br, the reaction is conveniently performed in the presence of a base, such as sodium hydroxide, in a suitable solvent, such as DMF. When W′ and X′ are an epoxide group, the reaction is conveniently performed in the presence of a base, such as sodium hydroxide, in a suitable solvent, such as DMF.

Compounds of formulae (II), (III) and (IV) are either known in the art or may be prepared by conventional methodology well known to one of ordinary skill in the art using, for instance, procedures described in the accompanying Schemes and Examples, or by alternative procedures which will be readily apparent.

Further details of suitable procedures will be found in the accompanying Schemes and Examples. For instance, compounds of formula (I) can be converted into other compounds of formula (I) using synthetic methodology well known in the art.

Thus, for example, the compound of formula (I) where D² is CCO₂alkyl may be converted into the compound of formula (I) where D² is CCO₂H by conversion of the ester to the carboxylic acid, for example, by treatment with KOH or NaOH in a suitable solvent, such as dioxane (optionally mixed with water), THF and/or methanol.

Furthermore, the compound of formula (I) where D² is CCO₂H may be converted into the compound of formula (I) where D² is CC(O)NR³R⁴ by reacting the carboxylic acid with HNR³R⁴ in the presence of a coupling reagent, such as HATU, and a base, such as diisopropylethylamine, in a solvent. Suitable solvents include DMF.

In addition, the compound of formula (I) where X or Y is C═O may be converted into the compound of formula (I) where X or Y is CH₂ by reduction of the oxo group with, for instance, a borane reagent, such as BH₃.THF, in a suitable solvent, such as THF.

Also, the compound of formula (I) where Q¹ is OH may be converted into the compound of formula (I) where Q¹ is O(CH₂)₀₋₃heteroaryl, O(CH₂)₀₋₃aryl or O(CH₂)₀₋₃C₃₋₈cycloalkyl by reacting the hydroxy group with hal-(CH₂)₀₋₃heteroaryl, hal-(CH₂)₀₋₃aryl or hal-(CH₂)₀₋₃C₃₋₈cycloalkyl respectively, where hal is a suitable halogen atom such as bromine or chlorine. The reaction is conveniently carried out in the presence of a base, such as sodium hydride, in a suitable solvent, such as DMF.

### General Synthetic Schemes

In general, five synthetic schemes may be used to obtain the compounds of formula (I).

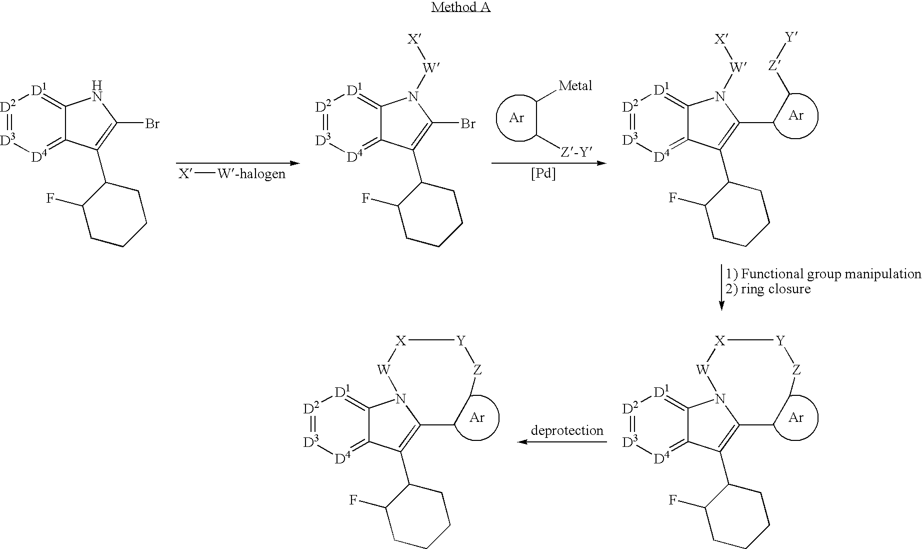

2-Bromoindole intermediate (prepared as described in Example 1) was functionalised on the indole nitrogen to introduce precursor functionality W′/X′ to either or both of the elements W/X of the tether. Pd mediated cross-coupling methodology (e.g., Suzuki, Stille, etc.) then brought in the C2 aromatic bearing precursor functionality Z′/Y′ to either or both of the elements Z/Y of the tether. Functional group manipulation followed by ring closure afforded the tetracyclic system. Ester deprotection then yielded the target indole carboxylic acids, with the C2 aromatic tethered to the indole nitrogen. The skilled person will readily understand that the order of the reactions may be reversed (i.e. Pd-mediated coupling followed by functionalisation on the indole nitrogen).

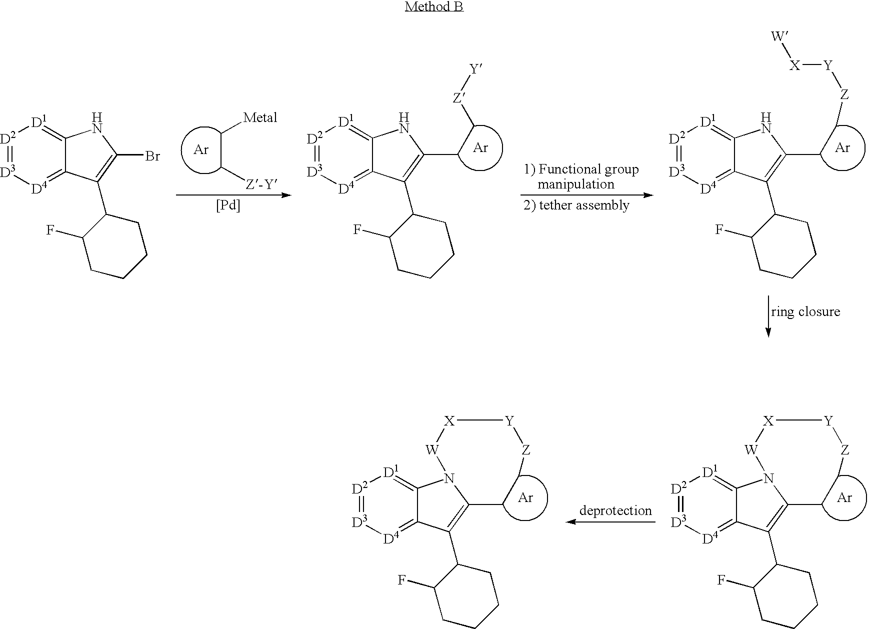

The C2 aromatic was introduced at the outset via Pd mediated cross-coupling methodology (Suzuki, Stille, etc.). The tether was then built up, with cyclisation onto the indole nitrogen finally closing the ring. Ester deprotection then yielded the target indole carboxylic acids, with the C2 aromatic tethered to the indole nitrogen.

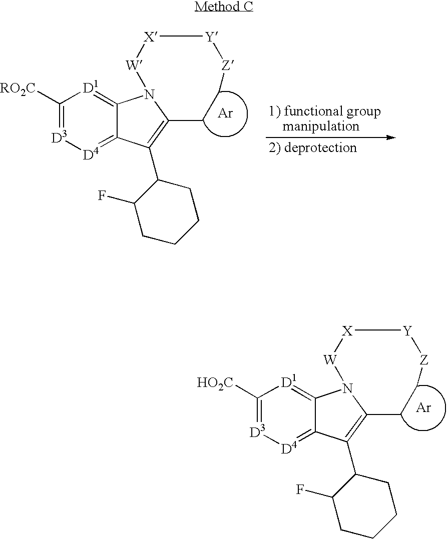

Fused tetracyclic intermediates arising from Methods A and B underwent manipulation of the functionality in the tether prior to ester deprotection to yield the target C2-tethered indole carboxylic acids.

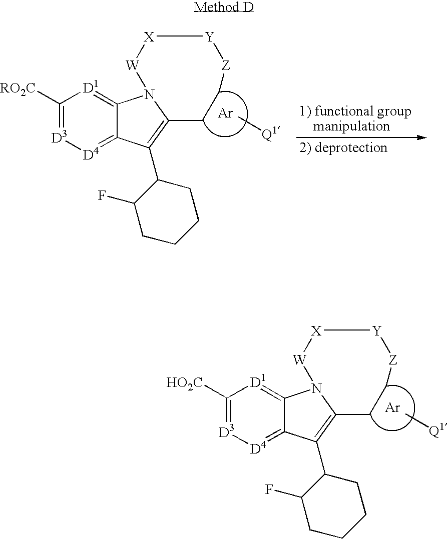

Fused tetracyclic intermediates arising from Methods A-C underwent manipulation of the functionality Q¹ on the Ar residue. This could be done before or after final functionalisation of the tether residues. Ester deprotection then yielded the target C2-tethered indole carboxylic acids.

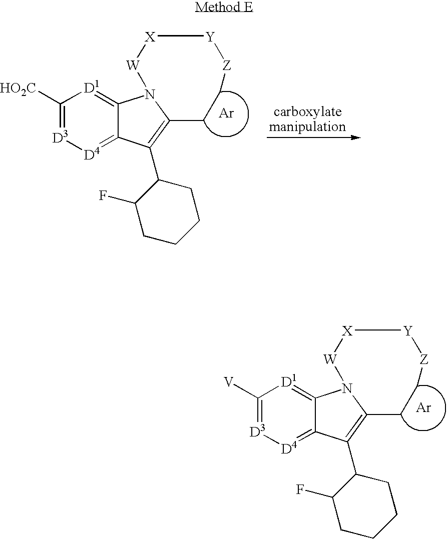

Tethered indole carboxylic acids arising from Methods A-D were further derivatised through manipulation of the carboxylate functionality to give compounds bearing a carboxylate replacement or carboxamide.

During any of the above synthetic sequences it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 3rd edition, 1999. The protecting groups may be removed at a convenient subsequent stage using methods known from the art.

The following Examples are illustrative of this invention.

The compounds of the invention were tested for inhibitory activity against the HCV RNA dependent RNA polymerase (NS5B) in an enzyme inhibition assay (example i)) and in a cell based sub-genomic replication assay (example ii)). The compounds have IC50's below 5 μM in the enzyme assay and several examples have EC50's below 2 μM in the cell based assay.

Compound names in the examples were generated using software from ACDLabs (version 6.0).

### i) In-Vitro HCV NS5B Enzyme Inhibition Assay

Published International patent application WO 96/37619 describes the production of recombinant HCV RdRp from insect cells infected with recombinant baculovirus encoding the enzyme. The purified enzyme was shown to possess in vitro RNA polymerase activity using RNA as template. The reference describes a polymerisation assay using poly(A) and oligo(U) as a primer or an heteropolymeric template. Incorporation of tritiated UTP or NTPs is quantified by measuring acid-insoluble radioactivity. This assay has been employed to screen the various compounds described above as inhibitors of HCV RdRp.

Incorporation of radioactive UMP was measured as follows. The standard reaction (50 μl) was carried out in a buffer containing 20 mM tris/HCl pH 7.5, 5 mM MgCl₂, 1 mM DTT, 50 mM NaCl, 0.03% N-octylglucoside, 1 μCi [³H]-UTP (40 Ci/mmol, NEN), 10 μM UTP and 10 μg/ml poly(A) or 5 μM NTPs and 5 μg/ml heteropolymeric template. Oligo(U)₁₂ (1 μg/ml, Genset) was added as a primer in the assay working on Poly(A) template. The final NS5B enzyme concentration was 5 nM. The order of assembly was: 1) compound, 2) enzyme, 3) template/primer, 4) NTP. After 1 h incubation at 22° C. the reaction was stopped by adding 50 μl of 20% TCA and applying samples to DE81 filters. The filters were washed thoroughly with 5% TCA containing 1M Na₂HPO₄/NaH₂PO₄, pH 7.0, rinsed with water and then ethanol, air dried, and the filter-bound radioactivity was measured in the scintillation counter. Carrying out this reaction in the presence of various concentrations of each compound set out above allowed determination of IC₅₀ values by utilising the formula:

% Residual activity=100/(1+[I]/IC₅₀)^{S}

where [I] is the inhibitor concentration and “s” is the slope of the inhibition curve.

### ii) Cell Based HCV Replication Assay

Cell clones that stably maintain subgenomic HCV replicon were obtained by transfecting Huh-7 cells with an RNA replicon identical to I₃₇₇neo/NS3-3′/wt described by Lohmann et al. (1999) (EMBL-genbank No. AJ 242652), followed by selection with neomycin sulfate (G418). Viral replication was monitored by measuring the expression of the NS3 protein by an ELISA assay performed directly on cells grown in 96 wells microtiter plates (Cell-ELISA) using the anti-NS3 monoclonal antibody 10E5/24 (as described in published International patent application WO02/59321). Cells were seeded into 96 well plates at a density of 10⁴ cells per well in a final volume of 0.1 ml of DMEM/10% FCS. Two hours after plating, 50 μl of DMEM/10% FCS containing a 3× concentration of inhibitor were added, cells were incubated for 96 hours and then fixed for 10′ with ice-cold isopropanol. Each condition was tested in duplicate and average absorbance values were used for calculations. The cells were washed twice with PBS, blocked with 5% non-fat dry milk in PBS+0.1% Triton X100+0.02% SDS (PBSTS) and then incubated overnight at 4° C. with the 10E5/24 mab diluted in Milk/PBSTS. After washing 5 times with PBSTS, the cells were incubated for 3 hours at room temperature with Fc specific anti-mouse IgG conjugated to alkaline phosphatase (Sigma), diluted in Milk/PBSTS. After washing again as above, the reaction was developed with p-Nitrophenyl phosphate disodium substrate (Sigma) and the absorbance at 405/620 nm read at intervals. For calculations, data sets were used where samples incubated without inhibitors had absorbance values comprised between 1 and 1.5. The inhibitor concentration that reduced by 50% the expression of NS3 (IC₅₀) was calculated by fitting the data to the Hill equation,

Fraction inhibition=1−(Ai−b)/(A₀−b)=[I]ⁿ/([I]ⁿ+IC₅₀)

where:
Ai=absorbance value of HBI10 cells supplemented with the indicated inhibitor concentration.
A₀=absorbance value of HBI10 cells incubated without inhibitor.
b=absorbance value of Huh-7 cells plated at the same density in the same microliter plates and incubated without inhibitor,
n=Hill coefficient.
   iii) General Procedures

All solvents were obtained from commercial sources (Fluka, puriss.) and were used without further purification. With the exception of routine deprotection and coupling steps, reactions were carried out under an atmosphere of nitrogen in oven dried (110° C.) glassware. Organic extracts were dried over sodium sulfate, and were concentrated (after filtration of the drying agent) on rotary evaporators operating under reduced pressure. Flash chromatography was carried out on silica gel following published procedure (W. C. Still et al., J. Org. Chem. 1978, 43, 2923) or on commercial flash chromatography systems (Biotage corporation and Jones Flashmaster II) utilising pre-packed columns.

Reagents were usually obtained directly from commercial suppliers (and used as supplied) but a limited number of compounds from in-house corporate collections were utilised. In the latter case the reagents were readily accessible using routine synthetic steps that are either reported in the scientific literature or are known to those skilled in the art.

¹H NMR spectra were recorded on Bruker AM series spectrometers operating at (reported) frequencies between 300 and 600 MHz. Chemical shifts (δ) for signals corresponding to non-exchangeable protons (and exchangeable protons where visible) are recorded in parts per million (ppm) relative to tetramethylsilane and are measured using the residual solvent peak as reference. Signals are tabulated in the order: multiplicity (s, singlet; d, doublet; t, triplet; q, quartet; m, multiplet; b, broad, and combinations thereof); coupling constants) in hertz (Hz); number of protons. Mass spectral (MS) data were obtained on a Perkin Elmer API 100, or Waters MicroMass ZQ, operating in negative (ES⁻) or positive (ES⁺) ionisation mode and results are reported as the ratio of mass over charge (m/z) for the parent ion only. Preparative scale HPLC separations were carried out on a Waters Delta Prep 4000 separation module, equipped with a Waters 486 absorption detector or on a Gilson preparative system. In all cases compounds were eluted with linear gradients of water and MeCN both containing 0.1% TFA using flow rates between 15 and 40 mL/min.

The following abbreviations are used in the examples, the schemes and the tables: Ac: acetyl; Ar: aryl; cat.: catalytic; dioxan(e): 1,4-dioxane; dppf: (1,1′-bisdiphenylphosphino)ferrocene; DAST: (diethylamino)sulfur trioxide]; 1,2-DCE: 1,2-dichloroethane; DIPEA: diisopropylethyl amine; DMAP: N,N-methylpyridin-4-amine; DME: dimethoxyethane; DMF: dimethylformamide; DMSO: dimethylsulfoxide; DMP: Dess-Martin Periodinane; EDAC.HCl: 1-ethyl-(3-dimethylaminopropyl)carbodiimide HCl salt; eq.: equivalents); Et₃N: triethylamine; EtOAc: ethyl acetate; Et₂O: diethyl ether, EtOH: ethanol; h: hour(s); Et₃SiH: triethylsilane; HOAc: acetic acid; HATU: O-(7-azabenzotriazol-1-yl)-N,N,N′,N′-tetramethyluronium hexafluorophosphate; Me: methyl; MeCN: acetonitrile; MeOH: methanol; min: minutes; MS: mass spectrum; NBS: N-bromo succinimide; PE: PE; Ph: phenyl; quant.: quantitative; RP-HPLC: reversed phase high-pressure liquid chromatography; RT: room temperature; sec: second(s); SFC: Super-critical fluid chromatography; s.s.: saturated aqueous solution; TBTU: O-benzotriazol-1-yl-N,N,N′,N′-tetramethyluronium tetrafluoroborate; TFA: trifluoroacetic acid; THF: tetrahydrofuran; THP: terhahydropyranyl; TMS: trimethylsilyl.

### EXAMPLE 1

### 14-[(1R,2S) or (1S,2R)-2-fluorocyclohexyl]-6-isopropyl-3-(pyridin-2-ylmethoxy)-5,6,7,8-tetrahydroindolo[2,1-a][2,5]benzodiazocine-11-carboxylic acid

### Step 1: methyl 3-cyclohex-1-en-1-yl-1H-indole-6-carboxylate

A solution (0.1 M) of 3-cyclohex-1-en-1-yl-1H-indole-6-carboxylic acid (prepared as described in published International patent application WO2004/087714) in dry DMF was cooled to 0° C. and treated with K₂CO₃ (1.05 eq). A solution (3 M) of MeI (1.05 eq) in DMF was then added over 0.5 h and the temperature was raised to 20° C. After 18 h the reaction was quenched with aqueous HCl (1 N) and diluted with EtOAc. The organic phase was separated and washed several times with aqueous HCl (1 N), then with brine. The dried organics were concentrated to give the title compound (99%) as a solid; MS m/z (ES⁺) 256 (M+H)⁺.

### Step 2: (±)-methyl 3-[(trans)-2-hydroxycyclohexyl]-1H-indole-6-carboxylate

A solution (0.2 M) of the preceding material in dry THF was treated over 1 h at 0° C. with BH₃.SMe₂ (2M in THF, 1.1 eq). The mixture was stirred at 20° C. for 12 h, then cooled to 0° C. and treated sequentially with aqueous NaOH (3 M, 5.7 eq) and H₂O₂ (30% in H₂O 8.4 eq). This mixture was stirred at 20° C. for 3 h then diluted with EtOAc and neutralized with s.s. NH₄Cl. The organic phase was washed with s.s. NaHCO₃ and brine, then dried and concentrated. The residue was washed several times with Et₂O to give the title compound (73%) as a white powder, MS m/z (ES⁺) 274 (M+H)⁺.

### Step 3: (±)-methyl 3-[(trans)-2-fluorocyclohexyl]-1H-indole-6-carboxylate

A solution (0.08 M) of the foregoing material in dry EtOAc was treated with DAST (1.2 eq) over 15 min at −50° C. The mixture was stirred for 1 h then warmed to 20° C. After 3 h the mixture was quenched with s.s. NaHCO₃ and diluted with EtOAc. The organic phase was washed with brine, dried and concentrated under reduced pressure. The residue was crystallized from hot EtOAc to give the title compound (61%). The filtrate was concentrated and the residue purified by flash chromatography (10% to 30% EtOAc: PE) to give a second crop of the title compound (17%) as a solid; MS m/z (ES⁺) 276 (M+H)⁺.

### Step 4: (±)-methyl 2-bromo-3-[(trans)-2-fluorocyclohexyl]-1H-indole-6-carboxylate

A solution (0.16 M) of the foregoing material in CH₂Cl₂ was treated with NBS (1.1 eq) over 2 h. The resulting mixture was stirred for 4 h then diluted with aqueous Na₂S₂O₃ (1 N) and stirred for 12 h. The organic phase was separated and washed with aqueous Na₂S₂O₃ (1 N) and brine. The dried organics were concentrated to afford a residue that was purified by flash chromatography (1:9 to 2:8 EtOAc:PE) to give the title compound (56%) as a pale solid; MS m/z (ES⁺) 354 (M+H)⁺.

### Step 5: methyl 2-bromo-3-[(1R,2S)-2-fluorocyclohexyl]-1H-indole-6-carboxylate and methyl 2-bromo-3-[(1S,2R)-2-fluorocyclohexyl]-1H-indole-6-carboxylate

The preceding material was dissolved in MeOH and the enantiomers were separated by SFC chromatography (stationary phase: Chiralcel OJ-H 250×10 mm; mobile phase: 25% MeOH containing 0.2% diethylamine/CO₂; flow rate 10 mL/min; column pressure: 100 bar, column temperature: 35° C.; detection UV 254 nm). The enantiomeric excess of the two fractions thus obtained (compound recovery 95%) were determined by chiral phase analytical HPLC (stationary phase: Chiralpak AD 250×4.6 mm; mobile phase 95:5 n-hexane:isopropyl alcohol containing 0.2% TFA; flow rate 1 mL/min; detection: UV 300 nM; sample concentration: 1 mg/mL; injection volume 10 uL): Isomer A (retention time 37.82 min, e.e. 99.8%, [α]_{D}²⁰=−8.0 (c=0.77, CHCl₃)); Isomer B (retention time 43.89 min, 99%, [α]_{D}²⁰=+8.0 (c=0.77, CHCl₃)).

### Step 6: methyl 2-bromo-1-(2-tert-butoxy-2-oxoethyl)-3-[(1R,2S) or (1S,2R)-2-fluorocyclohexyl]-1H-indole-6-carboxylate

A solution (0.16 M) of (−)-methyl 2-bromo-3-[(trans)-2-fluorocyclohexyl]-1H-indole-6-carboxylate (Isomer A from the preceding step) in dry DMF was cooled to 0° C. and treated with NaH (1.2 eq). After stirring for 1 h at RT, tert-butyl bromoacetate (1.1 eq) was added. After 2.5 h the reaction was quenched by addition of aqueous HCl (1 N) and diluted with EtOAc. The organic phase was separated then washed with aqueous HCl (1 N) and brine. The dried organics were concentrated to give a residue that was triturated with hexanes to afford the title compound (89%) as a white solid. ¹H NMR (300 MHz, DMSO-d₆, 300 K) δ 1.30-1.59 (m, 3H), 1.41 (s, 9H), 1.68-2.12 (m, 5H), 2.92-3.11 (m, 1H), 3.88 (s, 3H), 4.83-5.20 (m, 3H), 7.68 (d, J 8.4, 1H), 7.87 (d, J 8.4, 1H), 8.17 (s, 1H); [α]_{D}²⁰=−7.1 (c=1.0, MeOH).

### Step 7: methyl 2-[4-(benzyloxy)-2-formylphenyl]-1-(2-tert-butoxy-2-oxoethyl)-3-[(1R,2S) or (1S,2R)-2-fluorocyclohexyl]-1H-indole-6-carboxylate

A pyrex tube was charged with a solution (0.2 M) of the foregoing material in toluene. The solution was degassed then treated with [4-(benzyloxy)-2-formylphenyl]boronic acid (1.5 eq), 2-dicyclohexylphosphino-2′,6′-dimethoxybiphenyl (0.06 eq), K₃PO₄ (2 eq) and Pd₂(dba)₃ (0.02 eq). The tube was sealed and heated at 100° C. for 4.5 h, then the mixture was cooled and diluted with EtOAc and H₂O. The organic phase was separated then washed with brine and dried. Removal of the volatiles afforded a residue that was purified by flash chromatography (9:1 PE:EtOAc) to give the title compound (77%) as a solid. A 1:1* mixture of isomers were observed by ¹H NMR; MS (ES⁺) m/z 600 (M+H)⁺.

### Step 8: methyl 2-{-4-(benzyloxy)-2-[(isopropylamino)methyl]phenyl}-1-(2-tert-butoxy-2-oxoethyl)-3-[(1R,2S) or (1S,2R)-2-fluorocyclohexyl]-1H-indole-6-carboxylate

To a solution (0.03 M) of the foregoing material in THF was added i-PrNH₂ (10 eq) and AcOH (10 eq). The mixture was stirred for 12 h then the volatiles were removed in vacuo and the residue was taken up in MeOH. This solution (0.03 M) was treated with NaBH₃CN (1.05 eq) then stirred for 12 h and concentrated in vacuo. The residue was taken up in EtOAc, washed with s.s. NaHCO₃ and brine then dried. Removal of the solvent in vacuo afforded the title compound (95%) as a solid; MS (ES⁺) m/z 643 (M+H)⁺.

### Step 9: methyl 3-(benzyloxy)-14-[(1R,2S) or (1S,2R)-2-fluorocyclohexyl]-6-isopropyl-7-oxo-5,6,7,8-tetrahydroindolo[2,1-a][2,5]benzodiazocine-11-carboxylate

The foregoing material was treated with a 1:1 mixture of CH₂Cl₂:TFA and the resulting solution (0.05 M) was stirred for 6 h. The volatiles were removed in vacuo to afford a residue that was diluted with CH₂Cl₂. This solution (0.02M) was treated with DIPEA (2.5 eq) and HATU (1 eq) then stirred for 12 h. The volatiles were removed and the residue was diluted with EtOAc. The organic phase was washed with aqueous HCl (1 N), s.s. NaHCO₃ and brine then dried and concentrated to afford a residue that was purified by flash chromatography (3:2 PE/EtOAc) which afforded the title compound (76%) as a solid; MS (ES⁺) m/z 569 (M+H)⁺.

### Step 10: methyl 3-(benzyloxy)-14-[(1R,2S) or (1S,2R)-2-fluorocyclohexyl]-6-isopropyl-5,6,7,8-tetrahydroindolo[2,1-a][2,5]benzodiazocine-11-carboxylate

A solution (0.04 M) of the foregoing material in THF was treated with BH₃.THF (1 M solution in THF; 5 eq) and stirred for 2 h. The mixture was cooled to 0° C., diluted to 0.02 M by drop wise addition of MeOH then treated with methanolic HCl (1.25 M, 3 eq). The solution was heated to 65° C. for 2 h then cooled to 20° C. Removal of the volatiles in vacuo gave a residue that was diluted with EtOAc and s.s. NaHCO₃. The organic layer was washed with brine and dried then concentrated in vacuo to afford the title compound (97%) as a solid; MS (ES⁺) m/z 555 (M+H)⁺.

### Step 11: methyl 14-[(1R,2S) or (1S,2R)-2-fluorocyclohexyl]-3-hydroxy-6-isopropyl-5,6,7,8-tetrahydroindolo[2,1-a][2,5]benzodiazocine-11-carboxylate

A solution (0.01M) of the foregoing material in MeOH was treated with 10% Pd/C (10% by weight) and stirred under an atmosphere of hydrogen for 8 h. The mixture was filtered through celite and the filtrate was concentrated to give the title compound (93%) as a solid; MS (ES⁺) m/z 465 (M+H)⁺.

### Step 12: methyl 14-[(1R,2S) or (1S,2R)-2-fluorocyclohexyl]-6-isopropyl-3-(pyridin-2-ylmethoxy)-5,6,7,8-tetrahydroindolo[2,1-a][2,5]benzodiazocine-11-carboxylate

A solution (0.2 M) of the preceding material in DMF was cooled to 0° C. and treated portionwise with NaH (60% in mineral oil, 2.5 eq) and 2-(chloromethyl)pyridinium chloride (1.2 eq). The mixture was stirred for 5 h then diluted with EtOAc. The organic phase was washed with s.s. NaHCO₃ and brine then dried and concentrated to afford the title compound (98%) as a solid; MS (ES⁺) m/z 556 (M+H)⁺.

### Step 13: 14-[(1R,2S) or (1S,2R)-fluorocyclohexyl]-6-isopropyl-3-(pyridin-2-ylmethoxy)-5,6,7,8-tetrahydroindolo[2,1-a][2,5]benzodiazocine-11-carboxylic acid

A solution (0.03 M) of the foregoing material in a 1:1 mixture of dioxane:H₂O was treated with aqueous KOH (5 N, 2 eq) and heated at 40° C. for 48 h. The volatiles were removed in vacuo and the residue was acidified with aqueous HCl (1 N). This mixture was purified by RP-HPLC to afford a trifluoroacetate salt of the title compound (61%) that was identified as a 1:1* mixture of isomers by ¹H NMR. ¹H NMR (600 MHz, DMSO-d₆, 300 K) δ, 1.24-1.30 (m, 1H), 1.31 (d, J 6.3, 3H), 1.42 (d, J 5.1, 3H), 1.43-1.67 (m, 3H) 1.72-1.84 (m, 1.5H), 1.96-2.12 (m, 2H), 2.21-2.28 (m, 0.5H), 2.73-2.85 (m, 1H), 3.35-3.70 (m, partly obscured by residual H₂O signal, 3H), 3.80-3.90 (m, 2H), 4.46 and 4.54* (d, J and J* 13.8, 1H), 4.85-5.16 (m, 1H), 4.90 (d, J, J* 13.8, 1H), 5.26-5.39 (m, 1H), 7.37-7.46 (m, 3H), 7.47 and 7.57* (d, J and J* 8.5, 1H), 7.63 (d, J 7.7, 1H), 7.74 (d, J 8.5, 1H), 7.90 (t, J 7.7, 1H), 7.95 and 7.93* (d, J and J* 8.5, 1H), 8.20 (s, 1H), 8.63 (d, J 4.4, 1H), 9.35 (br s, 1H), 12.65 (br s, 1H); MS (ES⁺) m/z 542 (M+H)⁺.

### EXAMPLE 2

### 7-[[2-(dimethylamino)ethyl](methyl)amino]-14-[(1R,2S) or (1S,2R)-2-fluorocyclohexyl]-7,8-dihydro-6H-indolo[1,2-e][1,5]benzoxazocine-11-carboxylic acid

### Step 1: methyl 3-[(1R,2S) or (1S,2R)-2-fluorocyclohexyl]-2-(2-hydroxyphenyl)-1H-indole-6-carboxylate

Following the procedure described in Example 9, Step 4, treatment of a solution (0.16 M) of (−)-methyl 2-bromo-3-[(trans)-2-fluorocyclohexyl]-1H-indole-6-carboxylate (Isomer A, from Example 1, Step 5) with 2-hydroxyphenylboronic acid (1.8 eq), aqueous Na₂CO₃ (2 N, 4.6 eq) and Pd(PPh₃)₄ (0.1 eq) afforded a residue that was purified by flash chromatography (8:2 PE:EtOAc) to give the title compound (90%) as a solid. ¹H NMR (300 MHz, DMSO-d₆, 300 K) δ 1.21-1.65 (m, 3H), 1.68 (m, 4H), 2.05-2.19 (m, 1H), 2.75-2.97 (m, 1H), 3.87 (s, 3H), 5.00 (dm, J_{HF} 49.0, 1H), 6.93 (t, J 7.5, 1H), 7.01 (d, J 7.5, 1H), 7.28 (t, J 7.5, 1H), 7.29 (d, J 7.5, 1H), 7.59 (d, J 8.4, 1H), 7.82 (d, J 8.4, 1H), 8.02 (s, 1H), 9.74 (s, 1H), 11.34 (s, 1H).

### Step 2: methyl 3-[(1R,2S) or (1S,2R)-2-fluorocyclohexyl]-2-{2-[(2S)-oxiran-2-ylmethoxy]phenyl}-1H-indole-6-carboxylate

Following the procedure described in Example 10, Step 4, a solution (0.08 M) of the foregoing material was treated with CsF (6 eq) and (S)-glycidyl-3-nitrobenzenesulfonate (1.9 eq). The resulting residue was purified by flash chromatography (8:2 CH₂Cl₂: PE then CH₂Cl₂) to afford the title compound (68%) as a white solid. ¹H NMR (300 MHz, DMSO-d₆, 300 K) δ 1.12-2.20 (m, 8H), 2.54 (dd, partially obscured by signal from residual DMSO, J 4.8, 2.6, 1H), 2.66 (t, 74.8, 1H), 2.67-2.75 (m, 1H), 3.18-3.22 (m, 1H), 3.87 (s, 3H), 3.93 (dd, J 11.4, 5.6, 1H), 4.34 (dd, J 11.4, 2.7, 1H), 4.98 (dm, J_{HF} 49.3, 1H), 7.12 (i, J 7.5, 1H), 7.22 (d, J 7.5, 1H), 7.37 (d, J 7.5, 1H), 7.46 (t, J 7.5, 1H), 7.61 (d, J 8.4, 1H), 7.85 (d, J 8.4, 1H), 8.02 (s, 1H).

### Step 3: methyl (7S)-14-[(1R,2S) or (1S,2R)-2-fluorocyclohexyl]-7-hydroxy-7,8-dihydro-6H-indolo[1,2-e][1,5]benzoxazocine-11-carboxylate

Following the procedure described in Example 9, Step 6, a solution (0.02 M) of the foregoing material was treated with NaHMDS (12 eq) to afford a residue that was purified by flash chromatography (98:2 to 96:4 CH₂Cl₂:EtOAc) to give the title compound (35%) as a solid; MS m/z (ES⁺) 424 (M+H)⁺.

### Step 4: methyl (7R)-7-azido-14-[(1R,2S) or (1S,2R)-2-fluorocyclohexyl]-7,8-dihydro-6H-indolo[1,2-e][1,5]benzoxazocine-11-carboxylate

The preceding material was treated as described in Example 9, Step 7, to afford a residue that was purified by flash chromatography (1:3 CH₂Cl₂: PE then CH₂Cl₂) to give the title compound (91%) as a solid; MS m/z (ES⁺) 449 (M+H)⁺.

### Step 5: methyl (7R)-7-[(tert-butoxycarbonyl)amino]-14-[(1R,2S) or (1S,2R)-2-fluorocyclohexyl]-7,8-dihydro-6H-indolo[1,2-e][1,5]benzoxazocine-11-carboxylate

A solution (0.03 M) of the preceding material in an 8:3 mixture of dioxane:MeOH was treated with 10% Pd/C (10% by weight) and stirred under an atmosphere of H₂(g) for 1 h. The mixture was filtered through celite and the filtrate was concentrated to give a residue that was taken up in MeOH. This solution (0.02 M) was treated with TEA (2 eq) and Boc₂O (1.5 eq) then stirred for 12 h. After evaporation of the volatiles the residue was taken up in EtOAc, washed with aqueous HCl (1N), s.s. NaHCO₃ and brine then dried. Removal of the solvent in vacuo afforded the title compound as a solid (93%); MS (ES⁺) m/z 523 (M+H)⁺.

### Step 6: methyl (7R)-14-[(1R,2S)-2-fluorocyclohexyl]-7-(methylamino)-7,8-dihydro-6H-indolo[1,2-e][1,5]benzoxazocine-11-carboxylate

A solution (0.16 M) of the preceding material in DMF was cooled to 0° C. and treated portionwise with NaH (60% in mineral oil, 1.7 eq). The mixture was warmed to 20° C. then treated with dimethylsulfate (1.5 eq). After 3 h the mixture was diluted with EtOAc and aqueous HCl (1 N), then the organic phase was separated and washed with s.s. NaHCO₃ and brine then dried. Removal of the solvent afforded a residue that was taken up in an 8:2 mixture of DCM:TFA. This solution (0.07 M) was stirred for 1 h then the volatiles were removed in vacuo to give a residue that was taken up in EtOAc. The organic layer was washed with s.s. NaHCO₃ and brine then dried and concentrated to afford the title compound (72%) as a white solid; MS (ES⁺) m/z 437 (M+H)⁺.

### Step 7: methyl (7R)-7-[(2-aminoethyl)(methyl)amino]-14-[(1R,2S) or (1S,2R)-2-fluorocyclohexyl]-7,8-dihydro-6H-indolo[1,2-e][1,5]benzoxazocine-11-carboxylate

To a methanolic solution (0.02 M) of the preceding material was added AcOH (1.6 eq), tert-butyl(2-oxoethyl)carbamate (2.5 eq) and NaBH₃CN (2 eq). The mixture was stirred for 12 h then additional aldehyde (0.7 eq) and NaBH₃CN (0.5 eq) were added. After 12 h the volatiles were removed in vacuo and the residue was taken up in EtOAc. This solution was washed with s.s. NaHCO₃ and brine then dried. After evaporation of the volatiles the residue was purified by flash chromatography (8:2 to 6:4 PE:EtOAc) to give a white solid that was taken up in an 8:2 mixture of DCM:TFA. This solution (0.05M) was stirred for 1 h then the volatiles were removed in vacuo and the residue was taken up in EtOAc and washed with s.s. NaHCO₃ and brine. The dried organics were concentrated to afford the title compound (70%) as a white solid; MS (ES⁺) m/z 480 (M+H)⁺.

### Step 8: 7-[[2-(dimethylamino)ethyl](methyl)amino]-14-[(1R,2S) or (1S,2R)-2-fluorocyclohexyl]-7,8-dihydro-6H-indolo[1,2-e][1,5]benzoxazocine-11-carboxylic acid

To a methanolic solution (0.02 M) of the preceding material were added formaldehyde (3 eq), AcOH (1.6 eq) and NaBH₃CN (2 eq). The mixture was stirred for 0.5 h then the volatiles were removed in vacuo to give a residue that was taken up in EtOAc. This solution was washed with s.s. NaHCO₃ and brine then dried. After evaporation of volatiles the residue was dissolved in a 2:1 mixture of dioxane:H₂O. This solution (0.04 M) was treated with aqueous KOH (5 N, 2 eq) and heated to 65° C. for 12 h. The volatiles were removed in vacuo and the residue was acidified by addition of aqueous HCl (6 N). This mixture was purified by automated RP-HPLC to afford a trifluoroacetate salt of the title compound (55%) as a white solid. ¹H NMR (300 MHz, DMSO-d₆, 300 K) δ 1.19-1.40 (m, 2H), 1.45-1.62 (m, 1H), 1.70-1.92 (m, 2H), 1.96-2.18 (m, 3H), 2.38 (s, 3H), 2.89 (s, 6H), 2.90-3.01 (m, 1H), 3.05-3.23 (m, 3H), 3.31 (m, partially obscured by signal from residual H₂O, 2H), 3.88 (dd, J 14.3, 10.0, 1H), 4.07 (dd, J 11.9, 8.4, 1H), 4.27 (dd, J 11.9, 3.8, 1H), 4.70 (d, J 14.3, 1H), 5.01 (dm, J_{HF} 48.8, 1H), 7.25-7.36 (m, 3H), 7.55 (t, J 8.7, 1H), 7.70 (d, J 8.4, 1H), 7.93 (d, J 8.4, 1H), 8.20 (s, 1H); MS m/z (ES⁺) 494 (M+H)⁺; [α]_{D}²⁰=+16.0 (c=0.33, MeOH).

### EXAMPLE 3

### 14-[(1R,2S) or (1S,2R)-2-fluorocyclohexyl]-6-isopropyl-3-(pyridin-2-yloxy)-5,6,7,8-tetrahydroindolo[2,1-a][2,5]benzodiazocine-11-carboxylic acid

After standard cycles of evacuation and back-filling with nitrogen, an oven-dried Schlenk tube was charged with Cu₂O (0.05 eq), 2-hydroxybenzaldehyde oxime (0.2 eq) and Cs₂CO₃ (3 eq). A DMF solution (0.03 M) of methyl 14-[(1R,2S) or (1S,2R)-2-fluorocyclohexyl]-3-hydroxy-6-isopropyl-5,6,7,8-tetrahydroindolo[2,1-a][2,5]benzodiazocine-11-carboxylate (prepared as described in Example 1, Step 11) and 2-bromopyridine (1.5 eq) was added and the tube was sealed under a positive pressure of nitrogen. This mixture was stirred at 110° C. for 5 days then cooled and filtered through celite. The filtrate was concentrated to give a residue that was dissolved in a 2:1 mixture of dioxane:H₂O. This solution (0.05 M) was treated with aqueous KOH (5 N, 2 eq) and heated at 65° C. for 12 h. Aqueous KOH (5 N, 4 eq) was then added and stirring was continued for a further 24 h. The mixture was cooled and neutralized by addition of aqueous HCl (6 N). Purification of this mixture by RP-HPLC gave the title compound (12%) as a solid that was identified as a 1:1* mixture of isomers by ¹H NMR. ¹H NMR (300 MHz, DMSO-d₆, 300 K) δ 1.24-1.88 (m, 11.5H), 2.10-2.20 (m, 2H), 2.20-2.30 (m, 0.5H), 2.72-2.90 (m, 1H), 3.34-3.74 (m, 3H), 3.76-3.96 (m, 2H), 4.50 and 4.60* (d, J 14.1 and J* 13.0, 1H), 4.85-5.30 (m, 2H), 7.15-7.30 (m, 2H), 7.42-7.66 (m, 3H), 7.75 (d, J 8.2, 1H), 7.92-8.03 (m, 2H), 8.21-8.31 (m, 2H); MS (ES⁺) m/z 528 (M+H)⁺.

### EXAMPLE 4

### 6-[2-(dimethylamino)ethyl]-14-[(1S,2R)-2-fluorocyclohexyl]-3-methoxy-5,6,7,8-tetrahydroindolo[2,1-a][2,5]benzodiazocine-11-carboxylic acid and 6-[2-(dimethylamino)ethyl]-14-[(1R,2S)-2-fluorocyclohexyl]-3-methoxy-5,6,7,8-tetrahydroindolo[2,1-a][2,5]benzodiazocine-11-carboxylic acid

### Step 1: methyl 1-(2-tert-butoxy-2-oxoethyl)-3-[(1R,2S) or (1S,2R)-2-fluorocyclohexyl]-2-(2-formyl-4-methoxyphenyl)-1H-indole-6-carboxylate

Following the procedure described in Example 1, Step 7, treatment of (−)-methyl 2-bromo-1-(2-tert-butoxy-2-oxoethyl)-3-[(trans)-2-fluorocyclohexyl]-1H-indole-6-carboxylate prepared as described in Example 1, Step 6) with 2-formyl-4-methoxyphenylboronic acid (1.5 eq), aqueous Na₂CO₃ (2 N, 6 eq) and PdCl₂(PPh₃)₂ (0.2 eq) gave a residue that was purified by flash chromatography (9:1 to 8:2 PE:EtOAc) to give the title compound (68%) as a solid. This material was identified as a 1:1* mixture of isomers by ¹H NMR. ¹H NMR (300 MHz, DMSO-d₆, 300 K) δ 1.15-1.51 (m, 3H), 1.25 (s, 9H), 1.55-1.96 (m, 4H), 2.00-2.15 (m, 1H), 2.49-2.52 (m, obscured by residual signal from DMSO, 1H), 3.89 (s, 3H), 3.93 (s, 3H), 4.59 and 4.63* (d, J, J* 17.7, 1H), 4.88 and 4.89* (d, J, J* 17.7, 1H), 4.74-5.09 (m, 1H), 7.31-7.52 (m, 3H), 7.73 (d, J 8.4, 1H), 7.93 and 7.95* (d, J 8.4, 1H), 8.16 (s, 1H), 9.53 and 9.61* (s, 1H).

### Step 2: methyl 6-[2-(dimethylamino)ethyl]-14-[(1R,2S) or (1S,2R)-2-fluorocyclohexyl]-3-methoxy-7-oxo-5,6,7,8-tetrahydroindolo[2,1-a][2,5]benzodiazocine-11-carboxylate

A solution (0.05 M) of the preceding material in THF was treated with N,N-dimethylethane-1,2-diamine (10 eq) and AcOH (10 eq). The mixture was stirred for 2 h then concentrated in vacuo to give a residue that was taken up in MeOH. This solution (0.1 M) was treated with NaCNBH₃ (1.2 eq) and stirred for 12 h. The mixture was diluted with EtOAc and H₂O, and the organic layer was separated then washed with brine and dried. After removal of the solvent the residue was treated as described in Example 1, Step 9 with HATU (1.3 eq) and DIPEA (5 eq) to afford the title compound as a solid that was used directly in the subsequent step; MS m/z (ES⁺) 522 (M+H)⁺.

### Step 3: 6-[2-(dimethylamino)ethyl]-14-[(1R,2S) or (1S,2R)-2-fluorocyclohexyl]-3-methoxy-5,6,7,8-tetrahydroindolo[2,1-a][2,5]benzodiazocine-11-carboxylic acid

The preceding material was treated as described in Example 1, Steps 10 and 13 to furnish an enantiomer of the title compound (32%) as a white solid. This material was identified as a 1:1* mixture of isomers by ¹H NMR. ¹H NMR (300 MHz, DMSO-d₆, 300 K) δ 1.19-1.32 (m, 1H), 1.35-1.82 (m, 4.5H), 1.90-2.12 (m, 2H), 2.17-2.30 (m, 0.5H), 2.70-2.82 (m, 1H), 2.86 (s, 6H), 2.90-3.12 (m, 4H), 3.20-3.70 (m, partially obscured by signal from residual H₂O, 4H), 3.83 (d, J 12.9, 1H), 3.88 (s, 3H), 4.54 (d, J 12.9, 1H), 4.82-5.19 (m, 1H), 7.12 and 7.13* (d, J and J* 8.6, 1H), 7.24 and 7.25* (s, 1H), 7.33 and 7.43* (d, J and J* 8.6, 1H), 7.70 (d, J 8.4, 1H), 7.91 and 7.92* (d, J and J* 8.4, 1H), 8.11 (s, 1H); MS m/z (ES⁺) 494 (M+H)⁺.

Repetition of the procedure described in Steps 1-3 above using methyl 2-bromo-1-(2-tert-butoxy-2-oxoethyl)-3-[(1R,2S) or (1S,2R)-2-fluorocyclohexyl]-1H-indole-6-carboxylate that derives from Isomer B in Example 1, Step 5 afforded the other enantiomer of the title compound; MS m/z (ES⁺) 494 (M+H)⁺.

### EXAMPLE 5

### 6-[2-(dimethylammonio)ethyl]-14-[trans-2-fluorocyclohexyl]-11-(1H-tetrazol-5-yl)-5,6,7,8-tetrahydroindolo[2,1-a][2,5]benzodiazocin-6-ium bis(trifluoroacetate)

### Step 1: 6-[2-(dimethylamino)ethyl]-14-[(trans-2-fluorocyclohexyl]-5,6,7,8-tetrahydroindolo[2,1-a][2,5]benzodiazocine-11-carboxylic acid

Following the procedures described in Example 4, Steps 1-3, treatment of (+/−)-methyl 2-bromo-1-(2-tert-butoxy-2-oxoethyl)-3-[(trans)-2-fluorocyclohexyl]-1H-indole-6-carboxylate (prepared as described in Example 1, Step 7) with 2-formylphenylboronic acid afforded the title compound (25%) as a solid; MS (ES⁺) m/z 464 (M+H)⁺.

### Step 2: 11-(aminocarbonyl)-6-[2-(dimethylammonio)ethyl]-14-[trans-2-fluorocyclohexyl]-5,6,7,8-tetrahydroindolo[2,1-a][2,5]benzodiazocin-6-ium bis(trifluoroacetate)

To a solution (0.02 M) of the preceding material in DMF was added HATU (3.0 eq), aqueous NH₃ (20 eq) and DIPEA (6.0 eq). The reaction was stirred for 1.5 h at 20° C. then the solvent was removed under a stream of nitrogen. The residue was purified by RP-HPLC to afford a trifluoroacetate salt the title compound (85%) as a solid. A 1:1* mixture of isomers was observed in the ¹H NMR spectrum; MS (ES⁺) m/z (ES⁺) 463 (M+H)⁺.

### Step 3: 2-[14-[trans-2-fluorocyclohexyl]-11-(1H-tetrazol-5-yl)-7,8-dihydroindolo[2,1-a][2,5]benzodiazocin-6(5H)-yl]-N,N-dimethylethanamine

A solution (0.044 M) of the preceding material in anhydrous toluene was cooled to 0° C. then treated with Et₃N (20.0 eq) and TFA (10.0 eq). The reaction was warmed to 20° C. and followed by LCMS. After complete consumption of substrate the solvent was evaporated and the residue was diluted with EtOAc then washed with s.s. NaHCO₃ and brine. The dried organic layer was concentrated to give a residue that was taken up in toluene. This solution (0.02 M) was treated with Bu₃SnN₃ (6.0 eq) and stirred for 24 h at 110° C. Bu₃SnN₃ (2.7 eq) was added and stirring continued for 8 days. After evaporation of the solvent the residue was purified by RP-HPLC to afford the title compound (16%) as a solid. This material was identified as an approximately 1:1* mixture of isomers by ¹H NMR. ¹H NMR (600 MHz, DMSO-d₆, 300 K) δ 1.01-1.09 (m, 1H), 1.44-1.55 (m, 4H), 1.66-1.73 (m, 2H), 1.90-2.14 (m, 2H), 2.55-2.62 (m, 1H), 2.77 (s, 3H), 2.78 (s, 3H), 2.83-2.94 (m, 2H), 3.20-3.31 (m, 4H), 3.54 and 3.56* (d, J 15.5 and J* 15.5, 1H), 3.73, 3.74* (d, J 14.0, J* 14.0, 1H), 4.41 (d, J 15.5, 1H), 4.90 and 5.00* (dm, J_{HF} 49.0 and J*_{HF} 48.5, 1H), 7.41 and 7.51* (d, J and J* 7.5, 1H), 7.42 (t, 77.5, 1H), 7.48 (t, 77.5, 1H), 7.54 and 7.56* (d, J and J* 7.5, 1H), 7.69 (d, J 8.5, 1H), 7.97 and 7.98* (d, J and J* 8.5, 1H), 8.14 (s, 1H); ¹⁹F NMR (300 MHz, DMSO-d₆, 300 K) δ −169.2 and −171.32; MS (ES⁺) m/z 488 (M+H)⁺.

### EXAMPLE 6

### 6-ethyl-14-[(1R,2S) or (1S,2R)-2-fluorocyclohexyl]-3-(pyridin-2-ylmethoxy)-5,6,7,8-tetrahydroindolo[2,1-a][2,5]benzodiazocine-11-carboxylic acid

### Step 1: methyl 3-(benzyloxy)-6-ethyl-14-[(1R,2S) or (1S,2R)-2-fluorocyclohexyl]-7-oxo-5,6,7,8-tetrahydroindolo[2,1-a][2,5]benzodiazocine-11-carboxylate

Following the procedures described in Example 1, Steps 8-9, treatment of methyl 2-[4-(benzyloxy)-2-formylphenyl]-1-(2-tert-butoxy-2-oxoethyl)-3-[(1R,2S) or (1S,2R)-2-fluorocyclohexyl]-1H-indole-6-carboxylate (prepared as described in Example 1, Step 7) with EtNH₂ afforded a residue that was purified by flash chromatography (95:5 to 7:3 PE:EtOAc) to furnish the title compound (71%) as a solid. This material was identified as a 1:1* mixture of isomers by ¹H NMR; MS (ES⁺) m/z 555 (M+H)⁺.

### Step 2: methyl 6-ethyl-14-[(1R,2S) or (1S,2R)-2-fluorocyclohexyl]-3-hydroxy-5,6,7,8-tetrahydroindolo[2,1-a][2,5]benzodiazocine-11-carboxylate

The preceding material was treated at −78° C. with BH₃.THF (1 M solution in THF, 20.0 eq). The reaction was immediately warmed to 20° C. and after 2 h was quenched by cautious addition of MeOH. The mixture was heated to 80° C. for 2 h then cooled and diluted with EtOAc. The resulting solution (0.01 M) was treated with 10% Pd/C (10% by weight) and stirred under an atmosphere of hydrogen for 14 h. The solution was filtered and the filtrate concentrated in vacuo to afford the title compound (89%) as a solid; MS (ES⁺) m/z 451 (M+H)⁺.

### Step 3: 6-ethyl-14-[(1R,2S) or (1S,2R)-2-fluorocyclohexyl]-3-(pyridin-2-ylmethoxy)-5,6,7,8-tetrahydroindolo[2,1-a][2,5]benzodiazocine-11-carboxylic acid

A solution (0.03 M) of the preceding material in DMF was treated with NaH (60% suspension in mineral oil, 2.5 eq) then with 2-picolyl chloride hydrochloride salt (1.1 eq). After 3 h the DMF was removed under a stream of nitrogen and the residue treated with a 2:2:1 mixture of MeOH:dioxane:aqueous KOH (5 N). This solution (0.02 M) was heated to 80° C., stirred for 2 h, then cooled and acidified to pH 2. The residue was purified by RP-HPLC to furnish a trifluoroacetate salt of the title compound (41%) as a solid identified as a 1:1* mixture of isomers by ¹H NMR. ¹H NMR (600 MHz, DMSO-d₆, 300 K) δ 1.29 (br s, 3H), 1.36-1.46 (m, 2H), 1.53-1.55 (m, 3H), 1.65-1.72 (m, 2H), 1.89-1.99 and 2.14* (m, 2H), 2.67-2.74 (m, 1H), 3.28-3.40 (m, 3H), 3.52 and 3.60-3.72 (d, J 13.0, and m, 3H), 4.33 (t, J 15.0, 1H), 4.76 (d, J 15.0, 1H), 4.85-4.97 (m, 1H), 5.40 (br s, 2H), 7.31-7.34 (m, 1H), 7.43 (d, J 8.5, 0.5H), 7.46-7.50 (m, 1.5H), 7.62-7.70 (m, 2H), 7.82-7.86 (m, 2H), 8.14 (s, 1H), 8.20-8.21 (m, 1H), 8.74 (s, 1H), 9.56 (br s, 1H); ¹⁹F NMR (300 MHz, DMSO-d₆, 300 K) δ −169.0, −170.9; MS (ES⁺) m/z 528 (M+H)⁺.

### EXAMPLE 7

### 14-[(1R,2S) or (1S,2R)-2-fluorocyclohexyl]-6-isopropyl-3-(pyridin-3-ylmethoxy)-5,6,7,8-tetrahydroindolo[2,1-a][2,5]benzodiazocine-11-carboxylic acid

Following the procedure described in Example 1, Steps 12-13, treatment of methyl 14-[(1R,2S)-2-fluorocyclohexyl]-3-hydroxy-6-isopropyl-5,6,7,8-tetrahydroindolo[2,1-a][2,5]benzodiazocine-11-carboxylate (prepared as described in Example 1, Step 11) with NaH (3.3 eq), 3-(bromomethyl)pyridine hydrobromide (1.1 eq) and KOH (5 eq) gave a residue that was purified by RP-HPLC to afford a trifluoroacetate salt of the title compound (48%) as a solid. This material was identified as a 1:1* mixture of isomers by ¹H NMR. ¹H NMR (300 MHz, DMSO-d₆, 300 K) δ 1.08-1.17 (m, 2H), 1.31 (d, J 6.0, 3H), 1.42 (d, J 5.0, 3H), 1.49-1.65 (m, 3H), 1.71-1.88 (m, 0.5H), 1.97-2.09 (m, 2H), 2.20-2.32 (m, 0.5H), 2.75-2.81 (m, 1H), 3.30-3.40 (m, obscured by signal from residual H₂O, 2H), 3.79-3.90 (m, 4H), 4.47 and 4.56° (d, 0.7 and/13.5, 1H), 4.91-524 (m, 2H), 5.27-5.37 (m, 2H), 7.39-7.46 (m, 2H), 7.52-7.60 (m, 2H), 7.75 (d, J 8.5, 1H), 7.97 (t, J 4.0, 1H), 8.02 (d, J 8.5, 1H), 8.22 (s, 1H), 8.65 (d, J 4.0, 1H), 8.79 (s, 1H), 12.7 (br s, 1H); ¹⁹F NMR (300 MHz, DMSO-d₆, 300 K) δ −168.9, −170.7; MS (ES⁺) m/z 542 (M+H)₄; [α]_{D}²⁰=−4.8 (c=0.33, MeOH).

### EXAMPLE 8

### 14-[(1R,2S)-2-fluorocyclohexyl]-6-isopropyl-3-(pyridazin-3-ylmethoxy)-5,6,7,8-tetrahydroindolo[2,1-a][2,5]benzodiazocine-11-carboxylic acid

Following the procedure described in Example 1, Steps 12-13, treatment of methyl 14-[(1R,2S)-2-fluorocyclohexyl]-3-hydroxy-6-isopropyl-5,6,7,8-tetrahydroindolo[2,1-a][2,5]benzodiazocine-11-carboxylate (prepared as described in Example 1, Step 11) with NaH (3.3 eq) and 3-(chloromethyl)pyridazine (1.1 eq) and KOH (5 eq) gave a residue that was purified by RP-HPLC to afford a trifluoroacetate salt of the title compound (66%) as a solid. This material was identified as a 1:1* mixture of isomers by ¹H NMR. ¹H NMR (300 MHz, DMSO-d₆, 300 K) δ 1.08-1.17 (m, 2H), 1.31 (d, J 6.0, 3H), 1.45 (d, J 4.0, 3H), 1.49-1.67 (m, 2H), 1.71-1.86 (m, 2H), 1.97-2.09 (m, 2H), 2.74-2.82 (m, 1H), 3.30-3.40 (m, obscured by signal from residual H₂O, 1H), 3.33-3.68 (m, 4H), 4.48 and 4.57* (d, J and J* 13.5, 1H), 4.86-5.23 (m, 2H), 5.53-5.63 (br s, 2H), 7.41-7.51 and 7.58* (m and d, J* 8.5, 3H), 7.75 (d, J 8.5, 1H), 7.83 and 7.86* (d, J and J* 5, 1H), 7.95-7.99 (m, 2H), 8.23 (s, 1H), 9.30 (d, J 4.0, 1H); ¹⁹F NMR (300 MHz, DMSO-d₆, 300 K) δ −168.9, −170.7; MS (ES⁺) m/z 543 (M+H)⁺; [α]_{D}²⁰=−3.9 (c=0.33, MeOH).

### EXAMPLE 9

### (7R)-7-<dimethylamino)-14-[(1R,2S) or (1S,2R)-2-fluorocyclohexyl]-3-(pyridin-3-ylmethoxy)-7,8-dihydro-6H-indolo[1,2-e][1,5]benzoxazocine-11-carboxylic acid

### Step 1: 4-bromo-3-hydroxyphenyl 4-methylbenzenesulfonate

A solution (0.4 M) of 4-bromobenzene-1,3-diol in acetone was treated with K₂CO₃ (3.0 eq) and 4-methylbenzenesulfonyl chloride (1.1 eq). The mixture was heated under reflux for 20 h, then cooled and diluted with brine. The acetone was removed in vacuo and the residue was diluted with EtOAc then washed with 6N HCl and brine. The dried organic phase was concentrated in vacuo to afford a residue that was purified by flash chromatography (EtOAc:PE 2:8) to afford the title compound (70%) as a white solid. ¹H NMR (300 MHz, DMSO-d₆, 300 K) δ 2.44 (s, 3H), 6.39 (dd, J 8.6, 2.6, 1H), 6.67 (d, J 2.6, 1H), 7.48 (d, J 8.6, 1H), 7.50 (d, J 8.4, 2H), 7.56 (d, J 8.4, 2H), 10.77 (br s, 1H).

### Step 2: 3-(Benzyloxy)-4-bromophenyl 4-methylbenzenesulfonate

A solution (0.5 M) of the preceding compound in DMF was cooled to 0° C. then treated with NaH (60% in mineral oil, 1.8 eq). After 1 h, (bromomethyl)benzene (1.2 eq) was added and the reaction was stirred for 12 h before being diluted with EtOAc and washed with aqueous HCl (1 N) and brine. The organic layer was dried and concentrated to give a residue that was triturated with hexane/Et₂O to give the title compound (88%) as a white solid. ¹H NMR (400 MHz, DMSO-d₆, 300 K) δ 2.44 (s, 3H), 5.10 (s, 2H), 6.58 (dd, J 8.6, 2.5, 1H), 6.92 (d, J 2.5, 1H), 7.34-7.46 (m, 5H), 7.48 (d, J 8.3, 2H), 7.61 (d, J 8.6, 1H), 7.74 (d, J 8.3, 2H).

### Step 3: (2-(benzyloxy)-4-{[(4-methylphenyl)sulfonyl]oxy}phenyl)boronic acid

A solution (0.2 M) of the preceding material in a 3:1 mixture of toluene:THF was treated with B(OiPr)₃ (1.5 eq). The solution was cooled to −78° C. then treated dropwise over 0.5 h with n-BuLi (1.5 eq). After 2 h the solution was warmed to 20° C. then the reaction was quenched by addition of H₂O and EtOAc. The organic phase was separated, washed with H₂O and brine, then dried and concentrated in vacuo to afford an oil that was used directly in the subsequent step.

### Step 4: Methyl 2-(2-(benzyloxy)-4-{[(4-methylphenyl)sulfonyl]oxy}phenyl)-3-[(trans)-2-fluorocyclohexyl]-1H-indole-6-carboxylate

A solution (0.06 M) of (−)-methyl 2-bromo-3-[(trans)-2-fluorocyclohexyl]-1H-indole-6-carboxylate (prepared as described in Example 1, Step 5) in dioxane was treated with aqueous Na₂CO₃ (2 N, 6.0 eq), (2-(benzyloxy)-4-{[(4-methylphenyl)sulfonyl]oxy}phenyl)boronic acid (1.7 eq) and bis(triphenylphosphine)palladium(II) chloride (0.1 eq). The mixture was stirred at 80° C. for 2 h, then diluted with EtOAc, washed with aqueous HCl (1 N) and brine. The dried organic layer was concentrated in vacuo to give a residue that was crystallized from MeOH to afford the title compound (76%) as a pale solid. ¹H NMR (400 MHz, DMSO-d₆, 300 K) δ 1.05-1.20 (m, 1H), 1.22-1.39 (m, 2H), 1.39-1.52 (m, 2H), 1.52-1.69 (m, 2H), 1.72-1.89 (m, 2H), 2.43 (s, 3H), 2.57-2.663 (m, 1H), 3.85 (s, 3H), 4.78-5.03 (m, 1H), 5.03 (s, 2H), 6.76 (dd, J 8.3, 2.0, 1H), 6.93 (d, J 2.0, 1H), 7.22-7.31 (m, 5H), 7.32 (d, J 8.3, 1H), 7.48 (d, J 8.2, 2H), 7.58 (d, J 8.5, 1H), 7.76 (d, J 8.2, 2H), 7.80 (d, J 8.5, 1H), 7.97 (s, 1H), 11.48 (s, 1H).

### Step 5: Methyl 3-[(1R,2S) or (1S,2R)-2-fluorocyclohexyl]-2-{4-{[(4-methylphenyl)sulfonyl]oxy}-2-[(2S)-oxiran-2-ylmethoxy]phenyl}-1H-indole-6-carboxylate

A solution (0.1 M) of the preceding material in a 4:1 mixture of MeOH:dioxane was treated with 10% Pd/C (10% by weight) and then treated in a Parr reactor under 50 psi of hydrogen for 12 h. Additional 10% Pd/C (10% by weight) and 6 N HCl (2 eq) were added and treatment continued for a further 48 h. The mixture was filtered and the filtrate concentrated to give a residue that was taken up in DMF. This solution (0.1 M) was treated with CsF (3 eq), stirred for 0.5 h, then treated with solution (0.8 M) of (2S)-oxiran-2-ylmethyl 3-nitrobenzenesulfonate (1.3 eq) in DMF. The mixture was stirred for 12 h then diluted with EtOAc. The organic layer was washed with aqueous HCl (1 N), s.s. NaHCO₃) and brine. The dried organics were concentrated in vacuo and the residue was purified by flash chromatography to give the title compound (71%) as a solid; MS (ES⁺) m/z 594 (M+H)⁺.

### Step 6: Methyl (7S)-14-[(1R,2S) or (1S,2R)-2-fluorocyclohexyl]-7-hydroxy-3-{[(4-methylphenyl)sulfonyl]oxy}-7,8-dihydro-6H-indolo[1,2-e][1,5]benzoxazocine-11-carboxylate

A solution (0.02 M) of the preceding material in DMF was treated with NaHMDS (1.2 eq) for 1 h. The mixture was diluted with EtOAc and washed with aqueous HCl (1 N), s.s. NaHCO₃, and brine. The dried organics were concentrated to give a residue that was purified by flash chromatography to afford the title compound (60%) as a solid; MS (ES⁺) m/z 594 (M+H)⁺.

### Step 7: Methyl (7R)-7-azido-14-[(1R,2S) or (1S,2R)-2-fluorocyclohexyl]-3-hydroxy-7,8-dihydro-6H-indolo[1,2-e][1,5]benzoxazocine-11-carboxylate

A solution (0.17 M) of the preceding material in pyridine was treated with TsCl (2.5 eq) then stirred for 12 h. After dilution with EtOAc the organics were washed with aqueous HCl (1 N), s.s. NaHCO₃, and brine. The crude compound was taken up in THF and the resulting solution (0.03 M) was treated with TMSN₃ (3.5 eq) and tetrabutylammonium triphenyldifluorosilicate (3.5 eq) and then it was refluxed overnight. Further TMSN₃ (10 eq) was added over 5 days before being concentrated in vacuo. The residue was purified by flash chromatography to give the title compound as white powder (49%); MS (ES⁺) m/z 465 (M+H)⁺.

### Step 8: Methyl (7R)-7-azido-14-[(1R,2S) or (1S,2R)-2-fluorocyclohexyl]-3-(pyridin-3-ylmethoxy)-7,8-dihydro-6H-indolo[1,2-e][1,5]-benzoxazocine-11-carboxylate

A solution (0.04 M) of the preceding material in DMF was treated at 0° C. with NaH (60% by weight, 2.4 eq). After 10 min. the suspension was treated with 3-(bromomethyl)pyridine hydrobromide (1.2 eq) then the mixture was stirred for 1 h at 20° C. The reaction was quenched with H₂O and diluted with EtOAc then the organic phase was washed with and brine. The dried organics were concentrated to give a residue that was purified by flash chromatography to afford the title compound (83%) as a solid; MS (ES⁺) m/z 556 (M+H)⁺.

### Step 9: (7R)-7-(dimethylamino)-14-[(1R,2S) or (1S,2R)-2-fluorocyclohexyl]-3-(pyridin-3-ylmethoxy)-7,8-dihydro-6H-indolo[1,2-e][1,5]benzoxazocine-11-carboxylic acid

A solution (0.03 M) of the preceding material in CH₂Cl₂ was treated with PPh₃ (1.1 eq) then heated under reflux for 1 h. The volatiles were removed and the residue treated with a methanolic solution of ammonia (7 M). This solution (0.05 M) was heated under reflux for 5 days, then concentrated under reduced pressure to give a residue that was taken up in a 2:1 mixture of MeCN:MeOH. This solution (0.02 M) was treated with formaldehyde (5 eq), NaCNBH₃ (2.3 eq) and AcOH (1.1 eq) then stirred for 48 h. After dilution with EtOAc the organics were washed with s.s. NaHCO₃ and brine. The dried organic phase was concentrated and the residue was taken up in a 1:1 mixture of dioxane:H₂O. KOH (3 eq) was added and the mixture was stirred at 60° C. for 4 h, then cooled and quenched by addition of aqueous HCl (1 N). The mixture was purified by RP-HPLC to give the title compound (50%) as a solid. ¹H NMR (400 MHz, DMSO-d₆, 300 K) δ 1.04-1.20 (m, 1H), 1.38-1.70 (m, 4H), 1.72-1.88 (m, 1H), 1.92-2.10 (m, 1H), 2.20-2.30 (m, 1H), 2.81-2.92 (m, 1H), 2.99 (s, 3H), 3.85-3.92 (m, 1H), 4.05-4.22 (m, 2H), 4.38 (dd, J 6.0, 13.6, 1H), 5.00 (d, J 12.8, 1H), 5.01-5.25 (d, J 42.4, 1H), 526 (s, 2H), 6.93 (s, 1H), 7.00 (d, J 8.8, 1H), 7.40 (d, J 8.8, 1H), 7.60 (dd, J 5.0, 7.8, 1H), 7.72 (d, J 8.4, 1H), 7.94 (d, J 8.4, 1H), 8.06 (d, J 7.8, 1H), 8.34 (s, 1H), 8.66 (d, J 5.0, 1H), 8.78 (s, 1H); MS (ES⁺) m/z 544 (M+H)⁺

### EXAMPLE 10

### (7R and 7S)-7-[[2-dimethylamino)ethyl](methyl)amino]-14-[(1S,2R) or (1R,2S)-2-fluorocyclohexyl]-3-methoxy-7,8-dihydro-6H-indolo[1,2-c][1,5]benzoxazocine-11-carboxylic acid

### Step 1: Methyl 2-[2-(benzyloxy)-4-hydroxyphenyl]-3-[(1R,2S) or (1S,2R)-2-fluorocyclohexyl]-1H-indole-6-carboxylate

A solution (0.9 M) of methyl 2-(2-(benzyloxy)-4-{[(4-methylphenyl)sulfonyl]oxy}phenyl)-3-[(trans)-2-fluorocyclohexyl]-1H-indole-6-carboxylate (prepared as described in Example 9, Step 4) in MeOH was treated with NaOMe (5.0 eq) and was heated under reflux for 2 h. The mixture was cooled and diluted with aqueous HCl (1 N) then extracted with EtOAc. The EtOAc layer was washed with brine and dried. Removal of the volatiles gave a residue that was triturated with Et₂O to afford the title compound (99%) as a white solid. ¹H NMR (400 MHz, DMSO-d₆, 300K) δ 1.09-1.26 (m, 1H), 1.32-1.66 (m, 3H), 1.66-1.84 (m, 2H), 1.84-1.97 (m, 1H), 2.08-2.21 (m, 1H), 2.72-2.86 (m, 1H), 3.87 (s, 3H), 4.85-5.07 (m, 1H), 5.09 (s, 2H), 6.52 (dd, J 8.3, 2.0, 1H), 6.61 (d, J 2.0, 1H), 7.16 (d, J 8.3, 1H), 7.23-7.39 (m, 5H), 7.58 (dd, J 8.4, 1.4, 1H), 7.79 (d, J 8.4, 1H); 7.99 (d, J 1.4, 1H), 9.75 (s, 1H), 11.30 (s, 1H).

### Step 2: Methyl 2-[2-(benzyloxy)-4-methoxyphenyl]-3-[(1R,2S) or (1S,2R)-2-fluorocyclohexyl]-1H-indole-6-carboxylate

A solution (0.1 M) of the preceding material in DMF was cooled to 0° C. then treated with Cs₂CO₃ (1.05 eq) and MeI (1.05 eq). The mixture was stirred for 10 h, then diluted with EtOAc and washed with aqueous HCl (1 N) and brine. The dried organic layer was concentrated in vacuo to give a residue that was washed with Et₂O to afford the title compound (84%) as a pale solid; MS (ES⁺) m/z 488 (M+H)⁺.

### Step 3: Methyl 3-[(1R,2S) or (1S,2R)-2-fluorocyclohexyl]-2-(2-hydroxy-4-methoxyphenyl)-1H-indole-6-carboxylate

A solution (0.1 M) of the preceding material in a 3:1 mixture of dioxane/MeOH was treated with aqueous HCl (6 N, 0.3 eq) and 10% Pd/C (8% by weight). The mixture was stirred in a Parr reactor under 50 psi of hydrogen for 20 h. The mixture was filtered and the filtrate was concentrated in vacuo to give the title compound (99%) as a white solid; MS (ES⁺) m/z 398 (M+H)⁺.

### Step 4: Methyl 3-[(1R,2S) or (1S,2R)-2-fluorocyclohexyl]-2-{4-methoxy-2-[(2S)-oxiran-2-ylmethoxy]phenyl}-1H-indole-6-carboxylate

A solution (0.1 M) of the preceding material in DMF was treated with CsF (3.0 eq) and (2S)-oxiran-2-ylmethyl 3-nitrobenzenesulfonate (1.3 eq). The mixture was stirred at 20° C. for 12 h, then diluted with EtOAc and washed with H₂O and brine. The dried organic layer was concentrated in vacuo to give a residue that was purified by flash chromatography (CH₂Cl₂: EtOAc 98:2) to afford the title compound (88%) as a white solid; MS (ES⁺) m/z 454 (M+H)⁺.

### Step 5: Methyl 14-[(1R,2S) or (1S,2R)-2-fluorocyclohexyl]-3-methoxy-7-oxo-7,8-dihydro-6H-indolo[1,2-e][1,5]benzoxazocine-11-carboxylate

A solution (0.02 M) of the preceding material in DMF was treated with NaHMDS (1.2 eq). After 1 h the mixture was diluted with EtOAc and washed with aqueous HCl (1 N), s.s. NaHCO₃, and brine. The dried organic phase was concentrated in vacuo to give a residue that was purified by flash chromatography to afford methyl (7S)-14-[(1R,2S) or (1S,2R)-2-fluorocyclohexyl]-7-hydroxy-3-methoxy-7,8-dihydro-6H-indolo[1,2-e][1,5]benzoxazocine-11-carboxylate (30%); (ES⁺) m/z 454 (M+H)⁺.

A solution (0.06 M) of this material in CH₂Cl₂ was treated with DMP (1.1 eq) for 4 h then the reaction was quenched by addition of s.s. Na₂S₂O₄. The solution was diluted with EtOAc and then washed with a 1:1 mixture of s.s. Na₂S₂O₄ and s.s. NaHCO₃. The dried organic phase was concentrated under reduced pressure to give the title compound (90%) as a solid; MS (ES⁺) m/z 452 (M+H)⁺.

### Step 6: 7R and 7S-[[2-(dimethylamino)ethyl](methyl)amino]-14-[(1R,2S) or (1S,2R)-2-fluorocyclohexyl]-3-methoxy-7,8-dihydro-6H-indolo[1,2-e][1,5]benzoxazocine-11-carboxylic acid

A solution (0.05 M) of the preceding material in 1,2-DCE was treated with N,N-dimethylethane-1,2-diamine (5 eq), Na(OAc)₃BH (1.5 eq) and AcOH (2 eq). After 12 h the mixture was diluted with EtOAc and washed with s.s. NaHCO₃ and brine. The organic phases were dried and concentrated under reduced pressure to give a residue that was taken up in CH₂Cl₂. This solution (0.05 M) was treated with HCHO (4 eq), NaCNBH₃ (1.5 eq) and AcOH (1 eq) for 0.5 h then diluted with EtOAc and washed with s.s. NaHCO₃ and brine. The organic phases were dried and concentrated to give a residue that was dissolved in a 1:1 mixture of dioxane:H₂O (0.15 M). This solution (0.15 M) mixture was treated with aqueous KOH (5 N, 5 eq) then stirred at 70° C. for 1.5 h. The reaction was cooled and quenched with aqueous HCl (6 N) then diluted with DMSO and purified by RP-HPLC to give two diastereoisomers of the title compound. Isomer A (white solid, 40%) ¹H NMR (300 MHz, DMSO-d₆, 300 K) δ 1.15-1.40 (m, 2H), 1.41-1.61 (m, 1H), 1.70-1.88 (m, 2H), 1.92-2.15 (m, 3H), 2.41 (s, 3H), 2.80-2.90 (m, 1H), 2.84 (s, 6H), 3.06-3.23 (m, 4H), 3.30 (under water, m, 1H), 3.85 (s, 3H), 3.93 (d, J 10.4, 1H), 4.03 (m, 2H), 4.68 (d, J 13.5, 1H), 4.82-5.15 (dm, J_{HF} 49.3, 1H), 6.82 (d, J 2.4, 1H), 6.86 (dd, J 8.4, 2.4, 1H), 122 (d, J 8.4, 1H), 7.69 (d, J 8.4, 1H), 7.90 (d, J 8.4, 1H), 8.17 (s, 1H). [α]_{D}²⁰=+8.2 (c=0.33, MeOH); Analytical RP-HPLC (stationary phase: Xterra MS C18 5 μm 4.6×150 mm; mobile phase: isocratic 35% MeCN/H₂O (+0.1% TFA); flow rate 1 mL/min) retention time=17.5 min.; Isomer B (white solid, 16%). ¹H NMR (400 MHz, DMSO-d₆, 300 K) δ 1.04-1.18 (m, 1H), 1.35-1.71 (m, 4H), 1.72-1.85 (m, 1H), 1.94-2.10 (m, 1H), 2.38 (s, 3H), 2.83 (s, 6H), 2.83-2.95 (m, 1H), 3.05-3.25 (m, 4H), 3.30 (under water, m, 1H), 3.80-3.90 (m, 1H), 3.85 (s, 3H), 4.05-4.15 (m, 1H), 4.30 (d, J 8.0, 1H), 4.68 (d, J 8.0, 1H), 5.01-5.17 (dm, J_{HF} 48.8, 1H), 6.88 (s, 1H), 6.90 (d, J 8.6, 1H), 7.38 (d, J 8.6, 1H), 7.69 (d, J 8.4, 1H), 7.90 (d, J 8.4, 1H), 8.18 (s, 1H); (ES⁺) m/z 524 (M+H)⁺. [α]_{D}²⁰=−41.8 (c=0.33, MeOH); Analytical RP-HPLC (stationary phase: Xterra MS C18 5 μm 4.6×150 mm; mobile phase: isocratic 35% MeCN/H₂O (+0.1% TFA); flow rate 1 mL/min) retention time=19.8 min.

### EXAMPLE 11

### (2E)-3-{4-[({1-[({14-[(trans)-2-fluorocyclohexyl]-6-isopropyl-3-methoxy-5,6,7,8-tetrahydroindolo[2,1-a][2,5]benzodiazocin-11-yl}carbonyl)amino]cyclopentyl}carbonyl)amino]phenyl}acrylic acid

### Step 1: ethyl (2E)-3-(4-{[(1-aminocyclopentyl)carbonyl]amino}phenyl)acrylate trifluoroacetate

1-{[(benzyloxy)carbonyl]amino}cyclopentanecarboxylic acid was dissolved in DMF (0.2 M). HATU (1 eq) and triethylamine (3 eq) were added, followed by ethyl (2)-3-(4-aminophenyl)acrylate (0.95 eq). The resulting mixture was stirred for 48 h at 40° C. DMF was evaporated, the resulting oil taken up in EtOAc and the solution washed with aqueous HCl (1N), water, saturated aqueous NaHCO₃ and brine. Drying over NaHSO₄ and evaporation gave an orange solid, which was purified by flash chromatography on silica gel using PE/EtOAc (2.5:1, containing 1% EtOH) as the eluent. The resulting solid was dissolved at 0° C. with a 1:1 mixture of TFA:CH₂Cl₂, and the solution (0.1 M) was stirred for 2 h at RT. Evaporation gave a residue that was triturated with toluene to afford a solid that was used without further purification in the subsequent step. ¹H NMR (400 MHz, DMSO-d₆, 300 K) δ1.34 (t, J 6.9, 3H), 1.90-2.12 (m, 4H), 2.50-2.65 (m, 4H), 4.15 (q, J 6.9, 2H), 6.62 (d, J 16.0, 1H), 7.69 (d, J 16.0, 1H), 7.80 (br s, 2H), 8.35 (br s, 2H) 10.22 (s, 1H); MS (ES⁺) m/z 303 (M+H)⁺.

### Step 2: (2E)-3-{4-[({1-[({14-[(trans)-2-fluorocyclohexyl]-6-isopropyl-3-methoxy-5,6,7,8-tetrahydroindolo[2,1-a][2,5]benzodiazocin-11-yl}carbonyl)amino]cyclopentyl}carbonyl)amino]phenyl}acrylic acid

A solution (0.03 M) of the preceding material and 14-[trans-2-fluorocyclohexyl]-6-isopropyl-3-methoxy-5,6,7,8-tetrahydroindolo[2,1-a][2,5]benzodiazocine-11-carboxylic acid (1.0 eq, compound 160 in Table 1, prepared from methyl 1-(2-tert-butoxy-2-oxoethyl)-3-[(1R,2S) or (1S,2R)-2-fluorocyclohexyl]-2-(2-formyl-4-methoxyphenyl)-1H-indole-6-carboxylate (Example 4, Step 1) as described in Example 1, Steps 8-10 and 13) in CH₂Cl₂ was treated with HATU (1.2 eq) and DIPEA (3.0 eq) and stirred overnight at RT. The resulting residue was taken up in a 1:1 mixture of THF:H₂O and this solution (0.03 M) was treated with LiOH.H₂O (2 eq) then stirred at 50° C. for 12 h. The cooled solution was concentrated and acidified then purified by RP-HPLC to afford the title compound (9%) as a solid. ¹H NMR (300 MHz, DMSO-d₆, 300 K) δ 1.00-1.50 (m, 9H), 1.51-1.89 (m, 9H), 1.94-220 (m, 4H), 220-2.46 (m, 2H), 2.70-2.88 (m, 1H) 3.75-3.92 (m, 3H) 3.93 (s, 3H), 4.42-4.62 (m, 1H), 4.80-4.96 (m, 1H), 5.12 (dm J_{HF} 47.8, 1H), 6.43 (d, J 15.9, 1H), 7.25-7.36 (m, 2H), 7.54 (d, J 15.9, 1H), 7.60 (d, J 7.5, 2H), 7.66 (d, J 7.5, 2H), 7.71 (d, J 7.6, 1H), 7.97 (d, J 7.6, 1H), 8.18 (s, 1H), 8.40 (s, 1H), 9.32-9.60 (br s, 1H), 9.65-9.80 (br s, 1H), 11.40-13.00 (br s, 1H); MS (ES⁺) m/z 721 (M+H)⁺.

### EXAMPLE 12

### 7R and 7S-[2-(dimethylamino)ethyl]-14-[(1S,2R) or (1R,2S)-2-fluorocyclohexyl]-3-(pyridin-3-ylmethoxy)-7,8-dihydro-6H-indolo[1,2-e][1,5]benzoxazocine-11-carboxylic acid

### Step 1: 4-bromo-3-(methoxymethoxy)phenyl 4-methylbenzenesulfonate

A solution (0.85 M) of 4-bromo-3-hydroxyphenyl 4-methylbenzenesulfonate (prepared as described in Example 9, Step 1) in DMF was cooled to 0° C. then treated with NaH (60% in mineral oil, 1.2 eq). After 15 min, chloromethyl methyl ether (1.1 eq) was added and the reaction was stirred for 3 h before being diluted with EtOAc and washed with aqueous HCl (1 N), s.s NaHCO₃ and brine. The organic layer was dried and concentrated in vacuo. The residue was purified by flash chromatography to give the title compound as a colourless oil (95%); MS (ES⁺) m/z 409, 411 (M+Na)⁺.

### Step 2: (2-(methoxymethoxy)-4-{[(4-methylphenyl)sulfonyl]oxy}phenyl)boronic acid

A solution (0.1 M) of the preceding material in a 4:1 mixture of toluene:THF was treated with B(OiPr)₃ (1.5 eq). The solution was cooled to −78° C. then treated drop wise over 1 h with n-BuLi (1.5 eq). The solution was stirred at −78° C. for 2 h before warming to RT overnight. The reaction was quenched by addition of HCl (1 N) before diluting with EtOAc. The organic phase was separated, washed with s.s NaHCO₃ and brine, then dried and concentrated in vacuo to afford a pale yellow solid that was used directly in the subsequent step; MS (ES) m/z 351 (M−H)⁻.

### Step 3: methyl 3-[(1R,2S) or (1S,2R)-2-fluorocyclohexyl]-2-(2-(methoxymethoxy)-4-{[(4-methylphenyl)sulfonyl]oxy}phenyl)-1H-indole-6-carboxylate

A solution (0.2 M) of (−)-methyl 2-bromo-3-[(1R,2S) or (1S,2R)-2-fluorocyclohexyl]-1H-indole-6-carboxylate (prepared as described in Example 1, Step 5) in dioxane was treated with aqueous Na₂CO₃ (2N, 6.0 eq), (2-(methoxymethoxy)-4-{[(4-methylphenyl)sulfonyl]oxy}phenyl)boronic acid (2 eq) and bis(triphenylphosphine)palladium(II) dichloride (0.2 eq). The mixture was stirred at 90° C. for 2 h, then allowed to cool before being diluted with EtOAc, washed with aqueous HCl (1 N) and brine. The dried organic layer was concentrated in vacuo to give a residue that was purified by flash chromatography (EtOAc:PE 20:80) to afford the title compound (99%) as a pale yellow solid; MS (ES⁺) m/z 582 (M+H)⁺.

### Step 4: methyl 3-[(1R,2S) or (1S,2R)-2-fluorocyclohexyl]-2-(2-hydroxy-4-{[(4-methylphenyl)sulfonyl]oxy}phenyl)-1H-indole-6-carboxylate

A solution (0.05 M) of the preceding material in MeOH was treated with HCl (3 N, 2.0 eq). The mixture was stirred at 80° C. for 1 h, then allowed to cool before being diluted with EtOAc and washed with s.s. NaHCO₃ and brine. The dried organic layer was concentrated in vacuo to give a residue that was purified by flash chromatography (EtOAc:PE 25:75) to afford the title compound (89%) as a yellow solid; MS (ES⁺) m/z 538 (M+H)⁺.

### Step 5: methyl 14-[(1R,2S) or (1S,2R)-2-fluorocyclohexyl]-7-methylene-3-{[(4-methylphenyl)sulfonyl]oxy}-7,8-dihydro-6H-indolo[1,2-e][1,5]benzoxazocine-11-carboxylate

A solution (0.06 M) of the preceding material in DMF was treated with KOtBu (2.2 eq). After 15 min, 3-chloro-2-(chloromethyl)prop-1-ene (1.1 eq) was added and the reaction was stirred for 18 h before being diluted with EtOAc and washed with aqueous HCl (1 N) and brine. The organic layer was dried and concentrated in vacuo. The residue was purified by flash chromatography (EtOAc:PE 15:85) to give the title compound as a pale yellow gum (60%); MS (ES⁺) m/z 590 (M+H)⁺.

### Step 6: methyl 14-[(1R,2S) or (1S,2R)-2-fluorocyclohexyl]-7R and 7S-(hydroxymethyl)-3-{[(4-methylphenyl)sulfonyl]oxy}-7,8-dihydro-6H-indolo[1,2-e][1,5]benzoxazocine-11-carboxylate

A solution (0.07 M) of the preceding material in THF was cooled to 0° C. then treated dropwise with BH₃.DMS (2 M in THF, 3 eq). After warming to RT the solution was stirred for 2 h. The solution was cooled to 0° C. then treated dropwise with NaOH (1N) and HOOH (30 vol.) and stirred for 18 h. After dilution with EtOAc the organics were washed with s.s. NH₄Cl and brine. The organic layer was dried and concentrated in vacuo to give the title compound as a viscous oil (quantitative); MS (ES⁺) m/z 608 (M+H)⁺.

### Step 7: methyl 14-[(1R,2S) or (1S,2R)-2-fluorocyclohexyl]-3-{[(4-methylphenyl)sulfonyl]oxy}-7R and 7S-({[(4-methylphenyl)sulfonyl]oxy}methyl)-7,8-dihydro-6H-indolo[1,2-e][1,5]benzoxazocine-11-carboxylate

A solution (0.07 M) of the preceding material in pyridine/CH₂Cl₂ (1:1) was treated with TsCl (3 eq) then stirred for 72 h. After dilution with EtOAc the organics were washed with aqueous HCl (1 N), s.s. NaHCO₃, and brine. The organic layer was dried and concentrated in vacuo. The residue was purified by flash chromatography (EtOAc:PE 20:80) to give the title compound as a yellow oil (55%); MS (ES⁺) m/z 784 (M+Na)⁺.

### Step 8: methyl 7R and 7S-(cyanomethyl)-14-[(1R,2S or (1S,2R)-2-fluorocyclohexyl]-3-{[(4-methylphenyl)sulfonyl]oxy}-7,8-dihydro-6H-indolo[1,2-e][1,5]benzoxazocine-11-carboxylate

A solution (0.04 M) of the preceding material in DMF was treated with NaCN (1.2 eq) then stirred for 18 h. After dilution with EtOAc the organics were washed with s.s. NaHCO₃, and brine. The organic layer was dried and concentrated in vacuo. The residue was purified by flash chromatography (EtOAc:PE 25:75) to give the title compound as a colourless oil (55%); MS (ES⁺) m/z 617 (M+H)⁺.

### Step 9: methyl 7R and 7S-(cyanomethyl)-14-[(1R,2S) or (1S,2R)-2-fluorocyclohexyl]-3-hydroxy-7,8-dihydro-6H-indolo[1,2-e][1,5]benzoxazocine-11-carboxylate

A solution (0.03 M) of the preceding material in MeOH was treated with NaOMe (5 eq) and stirred at 80° C. for 1.5 h before being allowed to cool. AcOH was added to neutralize and the solution concentrated in vacuo to give the title compound as a viscous oil (quantitative); MS (ES⁺) m/z 485 (M+Na)⁺.

### Step 10: methyl 7R and 7S-(2-aminoethyl)-14-[(1R,2S) or (1S,2R)-2-fluorocyclohexyl]-3-hydroxy-7,8-dihydro-6H-indolo[1,2-e][1,5]benzoxazocine-11-carboxylate

A solution (0.04 M) of the preceding material in EtOH, acidified by addition of AcOH, was treated with 10% Pd/C (10% by weight) and stirred under a hydrogen atmosphere for 12 h. The hydrogen atmosphere was replaced with nitrogen, the mixture was filtered and the filtrate concentrated in vacuo to give the title compound (99%) as a viscous oil; MS (ES⁺) m/z 467 (M+H)⁺.

### Step 11: methyl 7R and 7S-[2-(dimethylamino)ethyl]-14-[(1R,2S) or (1S,2R)-2-fluorocyclohexyl]-3-hydroxy-7,8-dihydro-6H-indolo[1,2-e][1,5]benzoxazocine-11-carboxylate

A solution (0.02 M) of the preceding material in CH₂Cl₂ was treated with HCHO (37% aqueous, 6 eq) and stirred for 15 min before adding NaCNBH₃ (4 eq). The solution was stirred for 90 min then diluted with EtOAc and washed with s.s. NaHCO₃ and brine. The organics were dried and concentrated in vacuo to give an oil which was used directly without further purification; MS (ES⁺) m/z 495 (M+H)⁺.

### Step 12: methyl 7R and 7S-[2-(dimethylamino)ethyl]-14-[(1R,2S) or (1S,2R)-2-fluorocyclohexyl]-3-(pyridin-3-ylmethoxy)-7,8-dihydro-6H-indolo[1,2-e][1,5]benzoxazocine-11-carboxylate

A solution (0.07 M) of the preceding material in DMF was treated with NaH (60% in mineral oil, 3 eq). After 15 min, 3-(chloromethyl)pyridine hydrochloride (2 eq) was added and the reaction was stirred for 18 h before being diluted with EtOAc and washed with s.s NH₄Cl and brine. The organic layer was dried and concentrated in vacuo. The residue was purified by SCX cation column eluting with aminonia in methanol to give the title compound as a brown oil (48%); MS (ES⁺) m/z 586 (M+H)⁺.

### Step 13: 7R and 7S-[2-(dimethylamino)ethyl]-14-[(1R,2S) or (1S,2R)-2-fluorocyclohexyl]-3-(pyridin-3-ylmethoxy)-7,8-dihydro-6H-indolo[1,2-e][1,5]benzoxazocine-11-carboxylic acid

A solution (0.007 M) of the preceding material in MeOH was treated with aqueous NaOH (1N, 140 eq) and heated at 70° C. for 3 h before being allowed to cool. The solution was acidified with HCl (3 N) and the volatiles were removed in vacuo. This mixture was purified by RP-HPLC to afford the trifluoroacetate salt of the title compound (20%) that was characterised as a 1:1* mixture of isomers by ¹H NMR; ¹H NMR (400 MHz, DMSO-d₆+TFA, 300 K) δ 1.11-2.12 (m, 11H), 2.65-2.67 (m, 1H), 2.84 (s, 6H), 324-3.37 (m, 2H), 3.60-3.66 (m, 1H), 3.79-4.01 (m, 2H), 4.55-4.64 (m, 1H), 4.87-5.10 (m, 1H), 5.38 (s, 2H), 6.82-6.95 (m, 2H), 7.19 and 7.39* (d, J and J* 8.6, 1H), 7.69-7.70 (m, 1H), 7.86-7.89 (m, 1H), 7.99-8.00 (m, 1H), 8.14-8.16 (m, 1H), 8.51-8.53 (m, 1H), 8.85-8.86 (m, 1H), 8.98 (s, 1H); MS (ES⁺) m/z 572 (M+H)⁺.

### EXAMPLE 13

### 7R and 7S-[2-(dimethylamino)ethyl]-14-[(1R,2S) or (1S,2R)-2-fluorocyclohexyl]-3-methoxy-7,8-dihydro-6H-indolo[1,2-e][1,5]benzoxazocine-11-carboxylic acid

### Step 1: methyl 7R and 7S-(cyanomethyl)-14-[(1R,2S) or (1S,2R)-2-fluorocyclohexyl]-3-methoxy-7,8-dihydro-6H-indolo[1,2-e][1,5]benzoxazocine-11-carboxylate

A solution (0.1 M) of methyl 7-(cyanomethyl)-14-[(1R,2S) or (1S,2R)-2-fluorocyclohexyl]-3-hydroxy-7,8-dihydro-6H-indolo[1,2-e][1,5]benzoxazocine-11-carboxylate (prepared as described in Example 12, Step 9) in DMF was treated with Cs₂CO₃ (1.5 eq) and MeI (1.5 eq). The reaction was stirred for 72 h then diluted with EtOAc and washed with aqueous HCl (1 N) and brine. The organic phases were dried and concentrated in vacuo to give an oil which was used directly without further purification; MS (ES⁺) m/z All (M+H)⁺.

### Step 2: methyl 7R and 7S-[2-(dimethylamino)ethyl]-14-[(1R,2S) or (1S,2R)-2-fluorocyclohexyl]-3-methoxy-7,8-dihydro-6H-indolo[1,2-e][1,5]benzoxazocine-11-carboxylate

The preceding material was treated in analogous fashion to that described in Example 12, Steps 10 and 11 to furnish the title compound (28%) as a viscous oil; MS (ES⁺) m/z 509 (M+H)⁺.

### Step 3: 7R and 7S-[2-(dimethylamino)ethyl]-14-[(1R,2S) or (1S,2R)-2-fluorocyclohexyl]-3-methoxy-7,8-dihydro-6H-indolo[1,2-e][1,5]benzoxazocine-11-carboxylic acid

The preceding material was treated in analogous fashion to that described in Example 12, Step 13 to afford the trifluoroacetate salt of the title compound (10%); ¹H NMR (400 MHz, DMSO-d₆+TFA, 300 K) δ 1.15-2.08 (m, 11H), 2.65-2.67 (m, 1H), 2.83 (s, 6H), 3.61-3.64 (m, 1H), 3.83 (s, 3H), 3.80-4.06 (m, 3H), 4.55-4.67 (m, 1H), 4.80-5.23 (m, 2H), 6.67 (d, J 2.6, 1H), 6.78 (dd, J 8.6, 2.6, 1H), 7.15 (d, J 8.6, 1H), 7.68 (d, J 8.3, 1H), 7.90 (d, J 8.3, 1H), 8.16 (s, 1H); MS (ES⁺) m/z 495 (M+H)⁺.

### EXAMPLE 14

### 7R and 7S-[(dimethylamino)methyl]-14-[(1R,2S) or (1S,2R)-2-fluorocyclohexyl]-3-(pyridazin-3-ylmethoxy)-7,8-dihydro-6H-indolo[1,2-e][1,5]benzoxazocine-11-carboxylic acid

### Step 1: methyl 7R and 7S-[(dimethylamino)methyl]-14-[(1R,2S) or (1S,2R)-2-fluorocyclohexyl]-3-{[(4-methylphenyl)sulfonyl]oxy}-7,8-dihydro-6H-indolo[1,2-e][1,5]benzoxazocine-11-carboxylate

A solution (0.06 M) of methyl 14-[(1R,2S) or (1S,2R)-2-fluorocyclohexyl]-3-{[(4-methylphenyl)sulfonyl]oxy}-7R and 7S-({[(4-methylphenyl)sulfonyl]oxy}methyl)-7,8-dihydro-6H-indolo[1,2-e][1,5]benzoxazocine-11-carboxylate (prepared as described in Example 12, Step 7) in THF was treated with dimethylamine (35 eq) and the reaction heated in a sealed tube at 80° C. for 36 h before being allowed to cool. The mixture was concentrated in vacuo to give an oil which was used directly without further purification; MS (ES⁺) m/z 635 (M+H)⁺.

### Step 2: methyl 7R and 7S-[(dimethylamino)methyl]-14-[(1R,2S) or (1S,2R)-2-fluorocyclohexyl]-3-hydroxy-7,8-dihydro-6H-indolo[1,2-e][1,5]benzoxazocine-11-carboxylate

A solution (0.04 M) of the preceding material in MeOH was treated with NaOMe (5 eq) and stirred at 80° C. for 1.5 h before being allowed to cool. The methanol was removed in vacuo and the residue dissolved in EtOAc and washed with aqueous s.s. NH₄Cl and brine. The organic layer was dried and concentrated in vacuo to give the title compound as a pale yellow gum (88%); MS (ES⁻) m/z 479 (M−H)⁻.

### Step 3: methyl 7R and 7S-[(dimethylamino)methyl]-14-[(1R,2S) or (1S,2R)-2-fluorocyclohexyl]-3-(pyridazin-3-ylmethoxy)-7,8-dihydro-6H-indolo[1,2-e][1,5]benzoxazocine-11-carboxylate

A solution (0.1 M) of the preceding material in DMF was treated with NaH (60% in mineral oil, 2.2 eq). After 20 min, 3-(chloromethyl)pyridazine (1.2 eq, prepared from 3-methylpyridazine as described in international patent application WO 98/50385) was added and the reaction was stirred for 2 h before being diluted with EtOAc and washed with s.s NH₄Cl and brine. The organic layer was dried and concentrated in vacuo to give the title compound as a brown oil (96%); MS (ES⁺) m/z 573 (M+H)⁺.

### Step 4: 7R and 7S-[(dimethylamino)methyl]-14-[(1R,2S) or (1S,2R)-2-fluorocyclohexyl]-3-(pyridazin-3-ylmethoxy)-7,8-dihydro-6H-indolo[1,2-e][1,5]benzoxazocine-11-carboxylic acid

The preceding material was treated in analogous fashion to that described in Example 12, Step 13 to afford the trifluoroacetate salt of the title compound (20%) that was characterised as a 1:1* mixture of isomers by ¹H NMR; ¹H NMR (400 MHz, DMSO-d₆+TFA, 300 K) δ 1.10-224 (m, 10H), 2.78-2.84 (m, 1H), 2.88 (s, 6H), 3.22-3.32 (m, 1H), 3.64-3.95 (m, 3H), 4.74-5.07 (m, 2H), 5.49 (s, 2H), 6.80-6.86 (m, 1H), 6.88-6.95 (m, 1H), 7.16 and 7.37* (d, J and J* 8.6, 1H), 7.70 (d, J 8.3, 1H), 7.90 (m, 2H), 7.98 (d, J 8.3, 1H), 8.27 (s, 1H), 9.31 (dd, J 4.8, 1.5, 1H); MS (ES⁺) m/z 559 (M+H)⁺.

The following tables contain further examples:

TABLE 1

| Example | | | General |
|---|---|---|---|
| no. | Name | (M + H)⁺ | method |
| 101 | 6-[2-(dimethylamino)ethyl]-14-[trans-2-fluorocyclohexyl]-7-oxo- | 478 | A |
| | 5,6,7,8-tetrahydroindolo[2,1-a][2,5]benzodiazocine-11-carboxylic | | |
| | acid | | |
| 102 | 14-[trans-2-fluorocyclohexyl]-6-methyl-5,6,7,8- | 407 | A |
| | tetrahydroindolo[2,1-a][2,5]benzodiazocine-11-carboxylic acid | | |
| 103 | 6-[2-(dimethylamino)ethyl]-14-[trans-2-fluorocyclohexyl]-3- | 508 | A |
| | methoxy-7-oxo-5,6,7,8-tetrahydroindolo[2,1- | | |
| | a][2,5]benzodiazocine-11-carboxylic acid | | |
| 104 | 14-[trans-2-fluorocyclohexyl]-3-methoxy-6-methyl-5,6,7,8- | 436 | A |
| | tetrahydroindolo[2,1-a][2,5]benzodiazocine-11-carboxylic acid | | |
| 105 | 5-[trans-2-(dimethylamino)ethyl]-13-[(-2-fluorocyclohexyl]-6,7- | 450 | A/C |
| | dihydro-5H-indolo[1,2-d][1,4]benzodiazepine-10-carboxylic acid | | |
| 106 | 5-[2-(dimethylamino)-2-oxoethyl]-13-[trans-2-fluorocyclohexyl]-6- | 478 | A |
| | oxo-6,7-dihydro-5H-indolo[1,2-d][1,4]benzodiazepine-10- | | |
| | carboxylic acid | | |
| 107 | 6-[2-(dimethylamino)ethyl]-14-[trans-2-fluorocyclohexyl]-3- | 478 | A |
| | methyl-5,6,7,8-tetrahydroindolo[2,1-a][2,5]benzodiazocine-11- | | |
| | carboxylic acid | | |
| 108 | 3-(benzyloxy)-14-[trans-2-fluorocyclohexyl]-6-methyl-5,6,7,8- | 513 | A |
| | tetrahydroindolo[2,1-a][2,5]benzodiazocine-11-carboxylic acid | | |
| 109 | 3-(benzyloxy)-6-[2-(dimethylamino)ethyl]-14-[trans-2- | 570 | A |
| | fluorocyclohexyl]-5,6,7,8-tetrahydroindolo[2,1- | | |
| | a][2,5]benzodiazocine-11-carboxylic acid | | |
| 110 | 6-[2-(dimethylamino)ethyl]-14-[trans-2-fluorocyclohexyl]-N,3- | 491 | A |
| | dimethyl-5,6,7,8-tetrahydroindolo[2,1-a][2,5]benzodiazocine-11- | | |
| | carboxamide | | |
| 111 | 6-(N,N-dimethylglycyl)-14-[trans-2-fluorocyclohexyl]-3-methoxy- | 508 | A/C |
| | 5,6,7,8-tetrahydroindolo[2,1-a][2,5]benzodiazocine-11-carboxylic | | |
| | acid | | |
| 112 | 6-(N,N-dimethylglycyl)-14-[trans-2-fluorocyclohexyl]-3-methoxy- | 470 | A |
| | 5,6,7,8-tetrahydroindolo[2,1-a][2,5]benzodiazocine-11-carboxylic | | |
| | acid | | |
| 113 | 3-chloro-6-[2-(dimethylamino)ethyl]-14-[trans-2- | 498 | A |
| | fluorocyclohexyl]-5,6,7,8-tetrahydroindolo[2,1- | | |
| | a][2,5]benzodiazocine-11-carboxylic acid | | |
| 114 | 6-(N,N-dimethylglycyl)-14-[trans-2-fluorocyclohexyl]-5,6,7,8- | 478 | A/C |
| | tetrahydroindolo[2,1-a][2,5]benzodiazocine-11-carboxylic acid | | |
| 115 | (7S)-7-[2(dimethylamino)ethoxy]-14-[trans-2-fluorocyclohexyl]- | 481 | B |
| | 7,8-dihydro-6H-indolo[1,2-e][1,5]benzoxazocine-11-carboxylic | | |
| | acid | | |
| 116 | (7R)-7-[2-(dimethylamino)ethoxy]-14-[trans-2-fluorocyclohexyl]- | 481 | B/C |
| | 7,8-dihydro-6H-indolo[1,2-e][1,5]benzoxazocine-11-carboxylic | | |
| | acid | | |
| 117 | 3-(benzyloxy)-6-[2-(dimethylamino)ethyl]-N-(ethylsulfonyl)-14- | 661 | A/E |
| | [trans-2-fluorocyclohexyl]-5,6,7,8-tetrahydroindolo[2,1- | | |
| | a][2,5]benzodiazocine-11-carboxamide | | |
| 118 | 3-(benzyloxy)-6-[2-(dimethylamino)ethyl]-N-(ethylsulfonyl)-14- | 676 | A/E |
| | [trans-2-fluorocyclohexyl]-5,6,7,8-tetrahydroindolo[2,1- | | |
| | a][2,5]benzodiazocine-11-carboxamide | | |
| 119 | 6-[2-(dimethylamino)ethyl]-14-[trans-2-fluorocyclohexyl]-3- | 571 | A/D |
| | (pyridin-3-ylmethoxy)-5,6,7,8-tetrahydroindolo[2,1- | | |
| | a][2,5]benzodiazocine-11-carboxylic acid | | |
| 120 | 3-[2-(dimethylamino)ethoxy]-14-[trans-2-fluorocyclohexyl]-6- | 494 | A/D |
| | methyl-5,6,7,8-tetrahydroindolo[2,1-a][2,5]benzodiazocine-11- | | |
| | carboxylic acid | | |
| 121 | 6-[2-(dimethylamino)ethyl]-14-[trans-2-fluorocyclohexyl]-3-[(1- | 575 | A/D |
| | methyl-1H-1,2,4-triazol-3-yl)methoxy]-5,6,7,8- | | |
| | tetrahydroindolo[2,1-a][2,5]benzodiazocine-11-carboxylic acid | | |
| 122 | 6-[2-(dimethylamino)ethyl]-14-[trans-2-fluorocyclohexyl]-3-[(1- | 496 | A/C |
| | methyl-1H-1,2,4-triazol-3-yl)methoxy]-5,6,7,8- | | |
| | tetrahydroindolo[2,1-a][2,5]benzodiazocine-11-carboxylic acid | | |
| 123 | 3-fluoro-13-[trans-2-fluorocyclohexyl]-6-oxo-6,7-dihydro-5H- | 411 | A |
| | indolo[1,2-d][1,4]benzodiazepine-10-carboxylic acid | | |
| 124 | 3-fluoro-13-[trans-2-fluorocyclohexyl]-6-oxo-6,7-dihydro-5H- | 468 | A/C |
| | indolo[1,2-d][1,4]benzodiazepine-10-carboxylic acid | | |
| 125 | 3-fluoro-13-[trans-2-fluorocyclohexyl]-6-oxo-6,7-dihydro-5H- | 411 | A/C |
| | indolo[1,2-d][1,4]benzodiazepine-10-carboxylic acid | | |
| 126 | 3-fluoro-13-[trans-2-fluorocyclohexyl]-6,7-dihydro-5H-indolo[1,2- | 397 | A |
| | d][1,4]benzodiazepine-10-carboxylic acid | | |
| 127 | 6-(N,N-dimethylglycyl)-14-[trans-2-fluorocyclohexyl]-3-(pyridin- | 585 | A/C |
| | 3-ylmethoxy)-5,6,7,8-tetrahydroindolo[2,1-a][2,5]benzodiazocine- | | |
| | 11-carboxylic acid | | |
| 128 | 6-(N,N-dimethylglycyl)-14-[trans-2-fluorocyclohexyl]-3-(pyridin- | 585 | A/C |
| | 2-ylmethoxy)-5,6,7,8-tetrahydroindolo[2,1-a][2,5]benzodiazocine- | | |
| | 11-carboxylic acid | | |
| 129 | 6-(N,N-dimethylglycyl)-14-[trans-2-fluorocyclohexyl]-3-(pyridin- | 585 | A/C |
| | 4-ylmethoxy)-5,6,7,8-tetrahydroindolo[2,1-a][2,5]benzodiazocine- | | |
| | 11-carboxylic acid | | |
| 130 | 6-(N,N-dimethylglycyl)-3-ethoxy-14-[trans-2-fluorocyclohexyl]- | 522 | A/C/D |
| | 5,6,7,8-tetrahydroindolo[2,1-a][2,5]benzodiazocine-11-carboxylic | | |
| | acid | | |
| 131 | 3-ethoxy-14-[trans-2-fluorocyclohexyl]-6-methyl-5,6,7,8- | 451 | A/D |
| | tetrahydroindolo[2,1-a][2,5]benzodiazocine-11-carboxylic acid | | |
| 132 | 6-[2-(dimethylamino)ethyl]-3-ethoxy-14-[trans-2- | 508 | A |
| | fluorocyclohexyl]-5,6,7,8-tetrahydroindolo[2,1- | | |
| | a][2,5]benzodiazocine-11-carboxylic acid | | |
| 133 | 14-[trans-2-fluorocyclohexyl]-6-isopropyl-3-propoxy-5,6,7,8- | 493 | A |
| | tetrahydroindolo[2,1-a][2,5]benzodiazocine-11-carboxylic acid | | |
| 134 | 6-[2-(dimethylamino)ethyl]-14-[trans-2-fluorocyclohexyl]-3- | 522 | A |
| | propoxy-5,6,7,8-tetrahydroindolo[2,1-a][2,5]benzodiazocine-11- | | |
| | carboxylic acid | | |
| 135 | 14-[trans-2-fluorocyclohexyl]-3-propoxy-6-(2-pyrrolidin-1- | 548 | A |
| | ylethyl)-5,6,7,8-tetrahydroindolo[2,1-a][2,5]benzodiazocine-11- | | |
| | carboxylic acid | | |
| 136 | 14-[trans-2-fluorocyclohexyl]-6-(2-morpholin-4-ylethyl)-3- | 564 | A |
| | propoxy-5,6,7,8-tetrahydroindolo[2,1-a][2,5]benzodiazocine-11- | | |
| | carboxylic acid | | |
| 137 | 14-[trans-2-fluorocyclohexyl]-6-methyl-3-(pyridin-2-ylmethoxy)- | 514 | A/D |
| | 5,6,7,8-tetrahydroindolo[2,1-a][2,5]benzodiazocine-11-carboxylic | | |
| | acid | | |
| 138 | 6-[2-(diethylamino)ethyl]-14-[trans-2-fluorocyclohexyl]-3- | 599 | A/D |
| | (pyridin-2-ylmethoxy)-5,6,7,8-tetrahydroindolo[2,1- | | |
| | a][2,5]benzodiazocine-11-carboxylic acid | | |
| 139 | 14-[trans-2-fluorocyclohexyl]-3-(pyridin-2-ylmethoxy)-6-(2- | 597 | A/D |
| | pyrrolidin-1-ylethyl)-5,6,7,8-tetrahydroindolo[2,1- | | |
| | a][2,5]benzodiazocine-11-carboxylic acid | | |
| 140 | 14-[trans-2-fluorocyclohexyl]-6-methyl-3-(pyridin-3-ylmethoxy)- | 514 | A/D |
| | 5,6,7,8-tetrahydroindolo[2,1-a][2,5]benzodiazocine-11-carboxylic | | |
| | acid | | |
| 141 | 14-[trans-2-fluorocyclohexyl]-6-isopropyl-3-(pyridin-3- | 597 | A/D |
| | ylmethoxy)-5,6,7,8-tetrahydroindolo[2,1-a][2,5]benzodiazocine-11- | | |
| | carboxylic acid | | |
| 142 | 14-[trans-2-fluorocyclohexyl]-6-methyl-3-(pyridin-4-ylmethoxy)- | 514 | A/D |
| | 5,6,7,8-tetrahydroindolo[2,1-a][2,5]benzodiazocine-11-carboxylic | | |
| | acid | | |
| 143 | 6-[2-(dimethylamino)ethyl]-14-[trans-2-fluorocyclohexyl]-3- | 571 | A/D |
| | (pyridin-4-ylmethoxy)-5,6,7,8-tetrahydroindolo[2,1- | | |
| | a][2,5]benzodiazocine-11-carboxylic acid | | |
| 144 | 14-[trans-2-fluorocyclohexyl]-6-(2-morpholin-4-ylethyl)-3- | 613 | A/D |
| | (pyridin-4-ylmethoxy)-5,6,7,8-tetrahydroindolo[2,1- | | |
| | a][2,5]benzodiazocine-11-carboxylic acid | | |
| 145 | 3-ethoxy-14-[trans-2-fluorocyclohexyl]-6-isopropyl-5,6,7,8- | 479 | A |
| | tetrahydroindolo[2,1-a][2,5]benzodiazocine-11-carboxylic acid | | |
| 146 | 3-ethoxy-14-[trans-2-fluorocyclohexyl]-6-(2-pyrrolidin-1-ylethyl)- | 534 | A |
| | 5,6,7,8-tetrahydroindolo[2,1-a][2,5]benzodiazocine-11-carboxylic | | |
| | acid | | |
| 147 | 3-ethoxy-14-[trans-2-fluorocyclohexyl]-6-(2-morpholin-4-ylethyl)- | 550 | A |
| | 5,6,7,8-tetrahydroindolo[2,1-a][2,5]benzodiazocine-11-carboxylic | | |
| | acid | | |
| 148 | 14-[trans-2-fluorocyclohexyl]-6-methyl-3-propoxy-5,6,7,8- | 565 | A |
| | tetrahydroindolo[2,1-a][2,5]benzodiazocine-11-carboxylic acid | | |
| 149 | 3-(cyclopropylmethoxy)-6-[2-(dimethylamino)ethyl]-14-[trans-2- | 534 | A |
| | fluorocyclohexyl]-5,6,7,8-tetrahydroindolo[2,1- | | |
| | a][2,5]benzodiazocine-11-carboxylic acid | | |
| 150 | 3-(cyclopropylmethoxy)-14-[trans-2-fluorocyclohexyl]-6-(2- | 560 | A |
| | pyrrolidin-1-ylethyl)-5,6,7,8-tetrahydroindolo[2,1- | | |
| | a][2,5]benzodiazocine-11-carboxylic acid | | |
| 151 | 3-(cyclopropylmethoxy)-14-[trans-2-fluorocyclohexyl]-6-(2- | 576 | A |
| | morpholin-4-ylethyl)-5,6,7,8-tetrahydroindolo[2,1- | | |
| | a][2,5]benzodiazocine-11-carboxylic acid | | |
| 152 | 14-[trans-2-fluorocyclohexyl]-6-(2-morpholin-4-ylethyl)-3- | 613 | A/D |
| | (pyridin-2-ylmethoxy)-5,6,7,8-tetrahydroindolo[2,1- | | |
| | a][2,5]benzodiazocine-11-carboxylic acid | | |
| 153 | 6-[2-(diethylamino)ethyl]-14-[trans-2-fluorocyclohexyl]-3- | 599 | A/D |
| | (pyridin-3-ylmethoxy)-5,6,7,8-tetrahydroindolo[2,1- | | |
| | a][2,5]benzodiazocine-11-carboxylic acid | | |
| 154 | 14-[trans-2-fluorocyclohexyl]-6-(2-morpholin-4-ylethyl)-3- | 613 | A/D |
| | (pyridin-3-ylmethoxy)-5,6,7,8-tetrahydroindolo[2,1- | | |
| | a][2,5]benzodiazocine-11-carboxylic acid | | |
| 155 | 6-[2-(diethylamino)ethyl]-14-[trans-2-fluorocyclohexyl]-3- | 599 | A/D |
| | (pyridin-4-ylmethoxy)-5,6,7,8-tetrahydroindolo[2,1- | | |
| | a][2,5]benzodiazocine-11-carboxylic acid | | |
| 156 | 14-[trans-2-fluorocyclohexyl]-3-(pyridin-4-ylmethoxy)-6-(2- | 597 | A/D |
| | pyrrolidin-1-ylethyl)-5,6,7,8-tetrahydroindolo[2,1- | | |
| | a][2,5]benzodiazocine-11-carboxylic acid | | |
| 157 | 14-[trans-2-fluorocyclohexyl]-6-isopropyl-5,6,7,8- | 435 | A |
| | tetrahydroindolo[2,1-a][2,5]benzodiazocine-11-carboxylic acid | | |
| 158 | 14-[trans-2-fluorocyclohexyl]-6-(2-pyrrolidin-1-ylethyl)-5,6,7,8- | 490 | A |
| | tetrahydroindolo[2,1-a][2,5]benzodiazocine-11-carboxylic acid | | |
| 159 | 14-[trans-2-fluorocyclohexyl]-6-(2-morpholin-4-ylethyl)-5,6,7,8- | 506 | A |
| | tetrahydroindolo[2,1-a][2,5]benzodiazocine-11-carboxylic acid | | |
| 160 | 14-[trans-2-fluorocyclohexyl]-6-isopropyl-3-methoxy-5,6,7,8- | 465 | A |
| | tetrahydroindolo[2,1-a][2,5]benzodiazocine-11-carboxylic acid | | |
| 161 | 6-[2-(diethylamino)ethyl]-14-[trans-2-fluorocyclohexyl]-3- | 522 | A |
| | methoxy-5,6,7,8-tetrahydroindolo[2,1-a][2,5]benzodiazocine-11- | | |
| | carboxylic acid | | |
| 162 | 14-[trans-2-fluorocyclohexyl]-3-methoxy-6-(2-pyrrolidin-1- | 520 | A |
| | ylethyl)-5,6,7,8-tetrahydroindolo[2,1-a][2,5]benzodiazocine-11- | | |
| | carboxylic acid | | |
| 163 | 14-[trans-2-fluorocyclohexyl]-3-methoxy-6-(2-morpholin-4- | 536 | A |
| | ylethyl)-5,6,7,8-tetrahydroindolo[2,1-a][2,5]benzodiazocine-11- | | |
| | carboxylic acid | | |
| 164 | 3-fluoro-14-[trans-2-fluorocyclohexyl]-6-methyl-5,6,7,8- | 525 | A |
| | tetrahydroindolo[2,1-a][2,5]benzodiazocine-11-carboxylic acid | | |
| 165 | 3-fluoro-14-[trans-2-fluorocyclohexyl]-6-isopropyl-5,6,7,8- | 453 | A |
| | tetrahydroindolo[2,1-a][2,5]benzodiazocine-11-carboxylic acid | | |
| 166 | 6-[2-(dimethylamino)ethyl]-3-fluoro-14-[trans-2- | 482 | A |
| | fluorocyclohexyl]-5,6,7,8-tetrahydroindolo[2,1- | | |
| | a][2,5]benzodiazocine-11-carboxylic acid | | |
| 167 | 6-[2-(diethylamino)ethyl]-3-fluoro-14-[trans-2-fluorocyclohexyl]- | 510 | A |
| | 5,6,7,8-tetrahydroindolo[2,1-a][2,5]benzodiazocine-11-carboxylic | | |
| | acid | | |
| 168 | 3-fluoro-14-[trans-2-fluorocyclohexyl]-6-(2-pyrrolidin-1-ylethyl)- | 508 | A |
| | 5,6,7,8-tetrahydroindolo[2,1-a][2,5]benzodiazocine-11-carboxylic | | |
| | acid | | |
| 169 | 3-fluoro-14-[trans-2-fluorocyclohexyl]-6-(2-morpholin-4-ylethyl)- | 524 | A |
| | 5,6,7,8-tetrahydroindolo[2,1-a][2,5]benzodiazocine-11-carboxylic | | |
| | acid | | |
| 170 | 3-chloro-6-[2-(diethylamino)ethyl]-14-[trans-2-fluorocyclohexyl]- | 526 | A |
| | 5,6,7,8-tetrahydroindolo[2,1-a][2,5]benzodiazocine-11-carboxylic | | |
| | acid | | |
| 171 | 3-chloro-14-[trans-2-fluorocyclohexyl]-6-(2-pyrrolidin-1-ylethyl)- | 524 | A |
| | 5,6,7,8-tetrahydroindolo[2,1-a][2,5]benzodiazocine-11-carboxylic | | |
| | acid | | |
| 172 | 3-chloro-14-[trans-2-fluorocyclohexyl]-6-(2-morpholin-4-ylethyl)- | 540 | A |
| | 5,6,7,8-tetrahydroindolo[2,1-a][2,5]benzodiazocine-11-carboxylic | | |
| | acid | | |
| 173 | 2-fluoro-14-[trans-2-fluorocyclohexyl]-6-isopropyl-5,6,7,8- | 453 | A |
| | tetrahydroindolo[2,1-a][2,5]benzodiazocine-11-carboxylic acid | | |
| 174 | 6-[2-(dimethylamino)ethyl]-2-fluoro-14-[trans-2- | 582 | A |
| | fluorocyclohexyl]-5,6,7,8-tetrahydroindolo[2,1- | | |
| | a][2,5]benzodiazocine-11-carboxylic acid | | |
| 175 | 6-[2-(diethylamino)ethyl]-2-fluoro-14-[trans-2-fluorocyclohexyl]- | 510 | A |
| | 5,6,7,8-tetrahydroindolo[2,1-a][2,5]benzodiazocine-11-carboxylic | | |
| | acid | | |
| 176 | 2-fluoro-14-[trans-2-fluorocyclohexyl]-6-(2-pyrrolidin-1-ylethyl)- | 508 | A |
| | 5,6,7,8-tetrahydroindolo[2,1-a][2,5]benzodiazocine-11-carboxylic | | |
| | acid | | |
| 177 | 2-fluoro-14-[trans-2-fluorocyclohexyl]-6-(2-morpholin-4-ylethyl)- | 524 | A |
| | 5,6,7,8-tetrahydroindolo[2,1-a][2,5]benzodiazocine-11-carboxylic | | |
| | acid | | |
| 178 | 3-(benzyloxy)-14-[trans-2-fluorocyclohexyl]-6-isopropyl-5,6,7,8- | 541 | A |
| | tetrahydroindolo[2,1-a][2,5]benzodiazocine-11-carboxylic acid | | |
| 179 | 3-(benzyloxy)-6-[2-(diethylamino)ethyl]-14-[trans-2- | 598 | A |
| | fluorocyclohexyl]-5,6,7,8-tetrahydroindolo[2,1- | | |
| | a][2,5]benzodiazocine-11-carboxylic acid | | |
| 180 | 3-(benzyloxy)-14-[trans-2-fluorocyclohexyl]-6-(2-pyrrolidin-1- | 596 | A |
| | ylethyl)-5,6,7,8-tetrahydroindolo[2,1-a][2,5]benzodiazocine-11- | | |
| | carboxylic acid | | |

TABLE 2

| Example | | | General |
|---|---|---|---|
| no. | Name | (M + H)⁺ | method |
| 201 | 6-[2-(dimethylamino)ethyl]-4-fluoro-14-[trans-2- | 582 | A |
| | fluorocyclohexyl]-5,6,7,8-tetrahydroindolo[2,1 - | | |
| | a][2,5]benzodiazocine-11-carboxylic acid | | |
| 202 | 6-[2-(diethylamino)ethyl]-4-fluoro-14-[trans-2-fluorocyclohexyl]- | 510 | A |
| | 5,6,7,8-tetrahydroindolo[2,1-a][2,5]benzodiazocine-11-carboxylic | | |
| | acid | | |
| 203 | 4-fluoro-14-[trans-2-fluorocyclohexyl]-6-(2-pyrrolidin-1-ylethyl)- | 508 | A |
| | 5,6,7,8-tetrahydroindolo[2,1-a][2,5]benzodiazocine-11-carboxylic | | |
| | acid | | |
| 204 | 4-fluoro-14-[trans-2-fluorocyclohexyl]-6-(2-morpholin-4-ylethyl)- | 524 | A |
| | 5,6,7,8-tetrahydroindolo[2,1-a][2,5]benzodiazocine-11-carboxylic | | |
| | acid | | |
| 205 | 6-[2-(dimethylamino)ethyl]-14-[trans-2-fluorocyclohexyl]-7-oxo-3- | 585 | A/D |
| | (pyridin-2-ylmethoxy)-5,6,7,8-tetrahydroindolo[2,1- | | |
| | a][2,5]benzodiazocine-11-carboxylic acid | | |
| 206 | 3-chloro-6-[2-(dimethylamino)ethyl]-14-[trans-2- | 513 | A |
| | fluorocyclohexyl]-7-oxo-5,6,7,8-tetrahydroindolo[2,1- | | |
| | a][2,5]benzodiazocine-11-carboxylic acid | | |
| 207 | 6-[2-(dimethylamino)ethyl]-2-fluoro-14-[trans-2- | 496 | A |
| | fluorocyclohexyl]-7-oxo-5,6,7,8-tetrahydroindolo[2,1- | | |
| | a][2,5]benzodiazocine-11-carboxylic acid | | |
| 208 | 14-[trans-2-fluorocyclohexyl]-3-methoxy-6-(pyridin-4-ylmethyl)- | 514 | A/C |
| | 5,6,7,8-tetrahydroindolo[2,1-a][2,5]benzodiazocine-11-carboxylic | | |
| | acid | | |
| 209 | 14-[trans-2-fluorocyclohexyl]-3-methoxy-6-(pyridin-3-ylmethyl)- | 514 | A/C |
| | 5,6,7,8-tetrahydroindolo[2,1-a][2,5]benzodiazocine-11-carboxylic | | |
| | acid | | |
| 210 | 6-[2-(dimethylamino)ethyl]-14-[(1R,2S OR 1S,2R))-2- | 557 | A/D |
| | fluorocyclohexyl]-3-(pyridin-2-yloxy)-5,6,7,8-tetrahydroindolo[2,1- | | |
| | a][2,5]benzodiazocine-11-carboxylic acid | | |
| 211 | 3-chloro-6-(N,N-dimethylglycyl)-14-[trans-2-fluorocyclohexyl]- | 512 | A/C |
| | 5,6,7,8-tetrahydroindolo[2,1-a][2,5]benzodiazocine-11-carboxylic | | |
| | acid | | |
| 212 | 6-[2-(dimethylamino)ethyl]-14-[trans-2-fluorocyclohexyl]-3- | 571 | A/D |
| | (pyridin-2-ylmethoxy)-5,6,7,8-tetrahydroindolo[2,1- | | |
| | a][2,5]benzodiazocine-11-carboxylicacid | | |
| 213 | 6-[2-(diethylamino)ethyl]-3-ethoxy-14-[trans-2-fluorocyclohexyl]- | 536 | A |
| | 5,6,7,8-tetrahydroindolo[2,1-a][2,5]benzodiazocine-11-carboxylic | | |
| | acid | | |
| 214 | 5-[2-(dimethylamino)ethyl]-13-[trans-2-fluorocyclohexyl]-3- | 480 | A/D |
| | methoxy-6,7-dihydro-5H-indolo[1,2-d][1,4]benzodiazepine-10- | | |
| | carboxylic acid | | |
| 215 | 13-[trans-2-fluorocyclohexyl]-3-methoxy-5-methyl-6,7-dihydro- | 423 | A/D |
| | 5H-indolo[1,2-d][1,4]benzodiazepine-10-carboxylic acid | | |
| 216 | 14-[trans-2-fluorocyclohexyl]-3-methoxy-6-(1-methyl-L-prolyl)- | 534 | A/C |
| | 5,6,7,8-tetrahydroindolo[2,1-a][2,5]benzodiazocine-11-carboxylic | | |
| | acid | | |
| 217 | 6-[2-(dimethylamino)ethyl]-14-[trans-2-fluorocyclohexyl]-3- | 558 | A/D |
| | (pyrimidin-2-yloxy)-5,6,7,8-tetrahydroindolo[2,1- | | |
| | a][2,5]benzodiazocine-11-carboxylic acid | | |
| 218 | 5-[2-(dimethylamino)-2-oxoethyl]-13-[trans-2-fluorocyclohexyl]-3- | 508 | A/C |
| | methoxy-6-oxo-6,7-dihydro-5H-indolo[1,2-d][1,4]benzodiazepine- | | |
| | 10-carboxylic acid | | |
| 219 | (7R)-7-(dimethylamino)-14-[(1R,2R)-2-fluorocyclohexyl]-7,8- | 437 | B |
| | dihydro-6H-indolo[1,2-e][1,5]benzoxazocine-11-carboxylic acid | | |
| 220 | 14-[trans-2-fluorocyclohexyl]-6-isopropyl-3-(pyridin-2-yloxy)- | 528 | A/D |
| | 5,6,7,8-tetrahydroindolo[2,1-a][2,5]benzodiazocine-11-carboxylic | | |
| | acid | | |
| 221 | 6-ethyl-14-[(1R,2S OR 1S,2R)-2-fluorocyclohexyl]-3-(pyridazin-3- | 529 | A/D |
| | ylmethoxy)-5,6,7,8-tetrahydroindolo[2,1-a][2,5]benzodiazocine-11- | | |
| | carboxylic acid | | |
| 222 | 6-cyclopropyl-14-[(1R,2S OR 1S,2R)-2-fluorocyclohexyl]-3- | 541 | A/D |
| | (pyridazin-3-ylmethoxy)-5,6,7,8-tetrahydroindolo[2,1- | | |
| | a][2,5]benzodiazocine-11-carboxylic acid | | |
| 223 | 14-[(1R,2S OR 1S,2R)-2-fluorocyclohexyl]-6-propyl-3-(pyridazin- | 543 | A/D |
| | 3-ylmethoxy)-5,6,7,8-tetrahydroindolo[2,1-a][2,5]benzodiazocine- | | |
| | 11-carboxylic acid | | |
| 224 | 14-[(trans-2-fluorocyclohexyl]-6-isopropyl-3-[(1-oxidopyridin-2- | 544 | A/D |
| | yl)oxy]-5,6,7,8-tetrahydroindolo[2,1-a][2,5]benzodiazocine-11- | | |
| | carboxylic acid | | |
| 225 | 3-(benzyloxy)-5-[2-(dimethylamino)ethyl]-13-[trans-2- | 556 | A/C |
| | fluorocyclohexyl]-6,7-dihydro-5H-indolo[1,2- | | |
| | d][1,4]benzodiazepine-10-carboxylic acid | | |
| 226 | 6-ethyl-14-[(1R,2S OR 1S,2R)-2-fluorocyclohexyl]-3-(pyridin-3- | 528 | A/D |
| | ylmethoxy)-5,6,7,8-tetrahydroindolo[2,1-a][2,5]benzodiazocine-11- | | |
| | carboxylic acid | | |
| 227 | 6-ethyl-14-[(1R,2S OR 1S,2R)-2-fluorocyclohexyl]-3-(pyridin-4- | 528 | A/D |
| | ylmethoxy)-5,6,7,8-tetrahydroindolo[2,1-a][2,5]benzodiazocine-11- | | |
| | carboxylic acid | | |
| 228 | 6-ethyl-14-[(1R,2S OR 1S,2R)-2-fluorocyclohexyl]-3-(pyrimidin-2- | 529 | A/D |
| | ylmethoxy)-5,6,7,8-tetrahydroindolo[2,1-a][2,5]benzodiazocine-11- | | |
| | carboxylic acid | | |
| 229 | 14-[(1R,2S) OR (1S,2R)-2-fluorocyclohexyl]-6-isopropyl-3- | 542 | A/D |
| | (pyridin-4-ylmethoxy)-5,6,7,8-tetrahydroindolo[2,1- | | |
| | a][2,5]benzodiazocine-11-carboxylic acid | | |
| 230 | 14-[(1R,2S OR 1S,2R)-2-fluorocyclohexyl]-6-isopropyl-3-(pyrazin- | 529 | A/D |
| | 2-yloxy)-5,6,7,8-tetrahydroindolo[2,1-a][2,5]benzodiazocine-11- | | |
| | carboxylic acid | | |
| 231 | 6-ethyl-14-[(1R,2S OR 1S,2R))-2-fluorocyclohexyl]-3-(pyrazin-2- | 529 | A/D |
| | ylmethoxy)-5,6,7,8-tetrahydroindolo[2,1-a][2,5]benzodiazocine-11- | | |
| | carboxylic acid | | |
| 232 | 14-[(1R,2S OR 1S,2R))-2-fluorocyclohexyl]-6-propyl-3-(1,3- | 534 | A/D |
| | thiazol-2-yloxy)-5,6,7,8-tetrahydroindolo[2,1- | | |
| | a][2,5]benzodiazocine-11-carboxylic acid | | |
| 233 | (7R)-7-(dimethylamino)-14-[(1R,2S) or (1S,2R)-2- | 544 | B |
| | fluorocyclohexyl]-3-(pyridin-2-ylmethoxy)-7,8-dihydro-6H- | | |
| | indolo[1,2-e][1,5]benzoxazocine-11-carboxylic acid | | |
| 234 | indolo[2,1-a][2,5]benzodiazocine-11-carboxylic acid, 14-[(1R,2S) | 573 | A/D |
| | or (1S,2R)-2-fluorocyclohexyl]-5,6,7,8-tetrahydro-3-[(6-methoxy- | | |
| | 3-pyridazinyl)methoxy]-6-(1-methylethyl)- | | |
| 235 | 14-[(1R,2S) or (1S,2R)-2-fluorocyclohexyl]-6-isopropyl-3- | 543 | A/D |
| | (pyrimidin-4-ylmethoxy)-5,6,7,8-tetrahydroindolo[2,1- | | |
| | a][2,5]benzodiazocine-11-carboxylic acid | | |
| 236 | 14-[(1R,2S) or (1S,2R)-2-fluorocyclohexyl]-3-(pyrimidin-4- | 572 | A/D |
| | ylmethoxy)-6-(tetrahydrofuran-3-yl)-5,6,7,8-tetrahydroindolo[2,1- | | |
| | a][2,5]benzodiazocine-11-carboxylic acid | | |
| 237 | 14-[(1R,2S) or (1S,2R)-2-fluorocyclohexyl]-3-(pyridin-3- | 570 | A/D |
| | ylmethoxy)-6-(tetrahydrofuran-3-yl)-5,6,7,8-tetrahydroindolo[2,1- | | |
| | a][2,5]benzodiazocine-11-carboxylic acid | | |

TABLE 3

| Example | | | General |
|---|---|---|---|
| no. | Name | (M + H)⁺ | method |
| 301 | 7-{[[2-(dimethylamino)ethyl](methyl)amino]methyl}-14-[(1R,2S) | 615 | D |
| | or (1S,2R)-2-fluorocyclohexyl]-3-(pyridin-3-ylmethoxy)-7,8- | | |
| | dihydro-6H-indolo[1,2-e][1,5]benzoxazocine-11-carboxylic acid | | |
| 302 | 7-{[[2-(dimethylamino)ethyl](methyl)amino]methyl}-14-[(1R,2S) | 615 | D |
| | or (1S,2R)-2-fluorocyclohexyl]-3-(pyridin-2-ylmethoxy)-7,8- | | |
| | dihydro-6H-indolo[1,2-e][1,5]benzoxazocine-11-carboxylic acid | | |
| 303 | 7-{[[2-(dimethylamino)ethyl](methyl)amino]methyl}-14-[(1R,2S) | 616 | D |
| | or (1S,2R)-2-fluorocyclohexyl]-3-(pyridazin-3-ylmethoxy)-7,8- | | |
| | dihydro-6H-indolo[1,2-e][1,5]benzoxazocine-11-carboxylic acid | | |
| 304 | 7-{[[2-(dimethylamino)ethyl](methyl)amino]methyl}-14-[(1R,2S) | 629 | D |
| | or (1S,2R)-2-fluorocyclohexyl]-3-(1-pyridin-3-ylethoxy)-7,8- | | |
| | dihydro-6H-indolo[1,2-e][1,5]benzoxazocine-11-carboxylic acid | | |
| 305 | 7-{[[2-(dimethylamino)ethyl](methyl)amino]methyl}-14-[(1R,2S) | 629 | D |
| | or (1S,2R)-2-fluorocyclohexyl]-3-(1-pyridin-2-ylethoxy)-7,8- | | |
| | dihydro-6H-indolo[1,2-e][1,5]benzoxazocine-11-carboxylic acid | | |
| 306 | 7-{[[2-(dimethylamino)ethyl](methyl)amino]methyl}-14-[(1R,2S) | 630 | D |
| | or (1S,2R)-2-fluorocyclohexyl]-3-(1-pyridazin-3-ylethoxy)-7,8- | | |
| | dihydro-6H-indolo[1,2-e][1,5]benzoxazocine-11-carboxylic acid | | |
| 307 | 7-{[[2-(dimethylamino)ethyl](methyl)amino]methyl}-14-[(1R,2S) | 601 | D |
| | or (1S,2R)-2-fluorocyclohexyl]-3-(pyridin-2-yloxy)-7,8-dihydro- | | |
| | 6H-indolo[1,2-e][1,5]benzoxazocine-11-carboxylic acid | | |
| 308 | 7-{[[2-(dimethylamino)ethyl](methyl)amino]methyl}-14-[(1R,2S) | 538 | D |
| | or (1S,2R)-2-fluorocyclohexyl]-3-methoxy-7,8-dihydro-6H- | | |
| | indolo[1,2-e][1,5]benzoxazocine-11-carboxylic acid | | |
| 309 | 7-[(dimethylamino)methyl]-14-[(1R,2S) or (1S,2R)-2- | 558 | D |
| | fluorocyclohexyl]-3-(pyridin-3-ylmethoxy)-7,8-dihydro-6H- | | |
| | indolo[1,2-e][1,5]benzoxazocine-11-carboxylic acid | | |
| 310 | 7-[(dimethylamino)methyl]-14-[(1R,2S) or (1S,2R)-2- | 558 | D |
| | fluorocyclohexyl]-3-(pyridin-2-ylmethoxy)-7,8-dihydro-6H- | | |
| | indolo[1,2-e][1,5]benzoxazocine-11-carboxylic acid | | |
| 311 | 7-[(dimethylamino)methyl]-14-[(1R,2S) or (1S,2R)-2- | 572 | D |
| | fluorocyclohexyl]-3-(1-pyridin-3-ylethoxy)-7,8-dihydro-6H- | | |
| | indolo[1,2-e][1,5]benzoxazocine-11-carboxylic acid | | |
| 312 | 7-[(dimethylamino)methyl]-14-[(1R,2S) or (1S,2R)-2- | 572 | D |
| | fluorocyclohexyl]-3-(1-pyridin-2-ylethoxy)-7,8-dihydro-6H- | | |
| | indolo[1,2-e][1,5]benzoxazocine-11-carboxylic acid | | |
| 313 | 7-[(dimethylamino)methyl]-14-[(1R,2S) or (1S,2R)-2- | 573 | D |
| | fluorocyclohexyl]-3-(1-pyridazin-3-ylethoxy)-7,8-dihydro-6H- | | |
| | indolo[1,2-e][1,5]benzoxazocine-11-carboxylic acid | | |
| 314 | 7-[(dimethylamino)methyl]-14-[(1R,2S) or (1S,2R)-2- | 544 | D |
| | fluorocyclohexyl]-3-(pyridin-2-yloxy)-7,8-dihydro-6H-indolo[1,2- | | |
| | e][1,5]benzoxazocine-11-carboxylic acid | | |
| 315 | 7-[(dimethylamino)methyl]-14-[(1R,2S) or (1S,2R)-2- | 481 | D |
| | fluorocyclohexyl]-3-methoxy-7,8-dihydro-6H-indolo[1,2- | | |
| | e][1,5]benzoxazocine-11-carboxylic acid | | |
| 316 | 7-[2-(dimethylamino)ethyl]-14-[(1R,2S) or (1S,2R)-2- | 572 | D |
| | fluorocyclohexyl]-3-(pyridin-2-ylmethoxy)-7,8-dihydro-6H- | | |
| | indolo[1,2-e][1,5]benzoxazocine-11-carboxylic acid | | |
| 317 | 7-[2-(dimethylamino)ethyl]-14-[(1R,2S) or (1S,2R)-2- | 573 | D |
| | fluorocyclohexyl]-3-(pyridazin-3-ylmethoxy)-7,8-dihydro-6H- | | |
| | indolo[1,2-e][1,5]benzoxazocine-11-carboxylic acid | | |
| 318 | 7-[2-(dimethylamino)ethyl]-14-[(1R,2S) or (1S,2R)-2- | 586 | D |
| | fluorocyclohexyl]-3-(1-pyridin-3-ylethoxy)-7,8-dihydro-6H- | | |
| | indolo[1,2-e][1,5]benzoxazocine-11-carboxylic acid | | |
| 319 | 7-[2-(dimethylamino)ethyl]-14-[(1R,2S) or (1S,2R)-2- | 586 | D |
| | fluorocyclohexyl]-3-(1-pyridin-2-ylethoxy)-7,8-dihydro-6H- | | |
| | indolo[1,2-e][1,5]benzoxazocine-11-carboxylic acid | | |
| 320 | 7-[2-(dimethylamino)ethyl]-14-[(1R,2S) or (1S,2R)-2- | 587 | D |
| | fluorocyclohexyl]-3-(1-pyridazin-3-ylethoxy)-7,8-dihydro-6H- | | |
| | indolo[1,2-e][1,5]benzoxazocine-11-carboxylic acid | | |
| 321 | 7-[2-(dimethylamino)ethyl]-14-[(1R,2S) or (1S,2R)-2- | 558 | D |
| | fluorocyclohexyl]-3-(pyridin-2-yloxy)-7,8-dihydro-6H-indolo[1,2- | | |
| | e][1,5]benzoxazocine-11-carboxylic acid | | |
| 322 | 7-[2-(diethylamino)ethyl]-14-[(1R,2S) or (1S,2R)-2- | 600 | D |
| | fluorocyclohexyl]-3-(pyridin-3-ylmethoxy)-7,8-dihydro-6H- | | |
| | indolo[1,2-e][1,5]benzoxazocine-11-carboxylic acid | | |
| 323 | 7-[2-(diethylamino)ethyl]-14-[(1R,2S) or (1S,2R)-2- | 600 | D |
| | fluorocyclohexyl]-3-(pyridin-2-ylmethoxy)-7,8-dihydro-6H- | | |
| | indolo[1,2-e][1,5]benzoxazocine-11-carboxylic acid | | |
| 324 | 7-[2-(diethylamino)ethyl]-14-[(1R,2S) or (1S,2R)-2- | 601 | D |
| | fluorocyclohexyl]-3-(pyridazin-3-ylmethoxy)-7,8-dihydro-6H- | | |
| | indolo[1,2-e][1,5]benzoxazocine-11-carboxylic acid | | |
| 325 | 7-[2-(diethylamino)ethyl]-14-[(1R,2S) or (1S,2R)-2- | 614 | D |
| | fluorocyclohexyl]-3-(1-pyridin-3-ylethoxy)-7,8-dihydro-6H- | | |
| | indolo[1,2-e][1,5]benzoxazocine-11-carboxylic acid | | |
| 326 | 7-[2-(diethylamino)ethyl]-14-[(1R,2S) or (1S,2R)-2- | 614 | D |
| | fluorocyclohexyl]-3-(1-pyridin-2-ylethoxy)-7,8-dihydro-6H- | | |
| | indolo[1,2-e][1,5]benzoxazocine-11-carboxylic acid | | |
| 327 | 7-[2-(diethylamino)ethyl]-14-[(1R,2S) or (1S,2R)-2- | 615 | D |
| | fluorocyclohexyl]-3-(1-pyridazin-3-ylethoxy)-7,8-dihydro-6H- | | |
| | indolo[1,2-e][1,5]benzoxazocine-11-carboxylic acid | | |
| 328 | 7-[2-(diethylamino)ethyl]-14-[(1R,2S) or (1S,2R)-2- | 586 | D |
| | fluorocyclohexyl]-3-(pyridin-2-yloxy)-7,8-dihydro-6H-indolo[1,2- | | |
| | e][1,5]benzoxazocine-11-carboxylic acid | | |
| 329 | 7-[2-(diethylamino)ethyl]-14-[(1R,2S) or (1S,2R)-2- | 523 | D |
| | fluorocyclohexyl]-3-methoxy-7,8-dihydro-6H-indolo[1,2- | | |
| | e][1,5]benzoxazocine-11-carboxylic acid | | |
| 330 | 3-(benzyloxy)-7-{[[2-(dimethylamino)ethyl](methyl)amino] | 614 | D |
| | methyl}-14-[(1R,2S) or (1S,2R)-2-fluorocyclohexyl]-7,8-dihydro- | | |
| | 6H-indolo[1,2-e][1,5]benzoxazocine-11-carboxylic acid | | |
| 331 | 7-{[[2-(dimethylamino)ethyl](methyl)amino]methyl}-14-[(1R,2S) | 628 | D |
| | or (1S,2R)-2-fluorocyclohexyl]-3-(1-phenylethoxy)-7,8-dihydro- | | |
| | 6H-indolo[1,2-e][1,5]benzoxazocine-11-carboxylic acid | | |
| 332 | 3-(benzyloxy)-7-[(dimethylamino)methyl]-14-[(1R,2S) or (1S,2R)- | 557 | D |
| | 2-fluorocyclohexyl]-7,8-dihydro-6H-indolo[1,2- | | |
| | e][1,5]benzoxazocine-11-carboxylic acid | | |
| 333 | 7-[(dimethylamino)methyl]-14-[(1R,2S) or (1S,2R)-2- | 571 | D |
| | fluorocyclohexyl]-3-(1-phenylethoxy)-7,8-dihydro-6H-indolo[1,2- | | |
| | e][1,5]benzoxazocine-11-carboxylic acid | | |
| 334 | 3-(benzyloxy)-7-[2-(dimethylamino)ethyl]-14-[(1R,2S) or (1S,2R)- | 571 | D |
| | 2-fluorocyclohexyl]-7,8-dihydro-6H-indolo[1,2- | | |
| | e][1,5]benzoxazocine-11-carboxylic acid | | |
| 335 | 7-[2-(dimethylamino)ethyl]-14-[(1R,2S) or (1S,2R)-2- | 585 | D |
| | fluorocyclohexyl]-3-(1-phenylethoxy)-7,8-dihydro-6H-indolo[1,2- | | |
| | e][1,5]benzoxazocine-11-carboxylic acid | | |
| 336 | 3-(benzyloxy)-7-[2-(diethylamino)ethyl]-14-[(1R,2S) or (1S,2R)-2- | 599 | D |
| | fluorocyclohexyl]-7,8-dihydro-6H-indolo[1,2- | | |
| | e][1,5]benzoxazocine-11-carboxylic acid | | |
| 337 | 7-[2-(diethylamino)ethyl]-14-[(1R,2S) or (1S,2R)-2- | 613 | D |
| | fluorocyclohexyl]-3-(1-phenylethoxy)-7,8-dihydro-6H-indolo[1,2- | | |
| | e][1,5]benzoxazocine-11-carboxylic acid | | |

1. A compound of the formula (I): 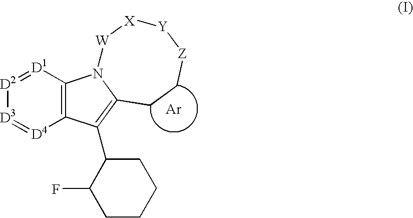
wherein
Ar is a moiety containing at least one aromatic ring and possesses 5-, 6-, 9- or 10-ring atoms optionally containing 1, 2 or 3 heteroatoms independently selected from N, O and S, which ring is optionally substituted at any substitutable position by groups Q¹ and Q²;
Q¹ is halogen, hydroxy, C₁₋₆alkyl, C₁₋₆alkoxy, aryl, heteroaryl, CONR^{c}R^{d}, (CH₂)₀₋₃NR^{c}R^{d}, O(CH₂)₀₋₃C₃₋₈cycloalkyl, O(CH₂)₁₋₃NR^{c}R^{d}, O(CH₂)₀₋₃CONR^{c}R^{d}, O(CH₂)₀₋₃aryl, OCH(CH₃)aryl, O(CH₂)₀₋₃heteroaryl, OCH(CH₃)heteroaryl or OCHR^{e}R^{f};
R^{c} and R^{d} are each independently selected from hydrogen, C₁₋₄alkyl and C(O)C₁₋₄alkyl;
or R^{c}, R^{d} and the nitrogen atom to which they are attached form a heteroaliphatic ring of 4 to 7 ring atoms, where said ring is optionally substituted by halogen, hydroxy, C₁₋₄alkyl or C₁₋₄alkoxy;
R^{e} and R^{f} are each independently selected from hydrogen and C₁₋₄alkoxy;
or R^{e} and R^{f} are linked by a heteroatom selected from N, O and S to form a heteroaliphatic ring of 4 to 7 ring atoms, where said ring is optionally substituted by halogen, hydroxy, C₁₋₄alkyl or C₁₋₄alkoxy;
and wherein said C₁₋₄alkyl, C₁₋₄alkoxy and aryl groups are optionally substituted by halogen or hydroxy;
Q² is halogen, hydroxy, C₁₋₄alkyl or C₁₋₄alkoxy, where said C₁₋₄alkyl and C₁₋₄alkoxy groups are optionally substituted by halogen or hydroxy;
or Q¹ and Q² may be linked by a bond or a heteroatom selected from N, O and S to form a ring of 4 to 7 atoms, where said ring is optionally substituted by halogen, hydroxy, C₁₋₄alkyl or C₁₋₄alkoxy;
D¹ is N or CR^{a};
D² is Nor CR¹;
D³ is N or CR²;
D⁴ is N or CR^{b};
with the proviso that D² and D³ are not both N;
R^{a} and R^{b} are each independently selected from hydrogen, fluorine, chlorine, C₁₋₄alkyl, C₂₋₄alkenyl or C₁₋₄alkoxy, where said C₁₋₄alkyl, C₂₋₄alkenyl and C₁₋₄alkoxy groups are optionally substituted by hydroxy or fluorine;
one of R¹ or R² is hydrogen, halogen, C₁₋₄alkyl, C₁₋₄alkoxy, CN, CO₂H, CO₂C₁₋₄alkyl, aryl, heteroaryl or C(O)NR³R⁴, where said C₁₋₄alkyl, C₁₋₄alkoxy, aryl and heteroaryl groups are optionally substituted by hydroxy or fluorine;
R³ is hydrogen or C₁₋₄alkyl;
R⁴ is hydrogen, C₁₋₄alkyl, C₂₋₄alkenyl, (CH₂)₀₋₃R⁵, SO₂R⁶ or -L-CO₂R²⁰;
R⁵ is NR^{h}Rⁱ, OR^{h}, aryl, heteroaryl or Het;
R^{h} and Rⁱ are each independently selected from hydrogen and C₁₋₄alkyl;
Het is a heteroaliphatic ring of 4 to 7 ring atoms, which ring may contain 1, 2 or 3 heteroatoms selected from N, O or S or a group S(O), S(O)₂, NH or NC₁₋₄alkyl;
R⁶ is C₁₋₄alkyl, C₂₋₄alkenyl or (CH₂)₀₋₃R⁷;
R⁷ is aryl, heteroaryl, C₁₋₄alkyl, C₃₋₈cycloalkyl, CO₂R⁸, Het or NR^{m}Rⁿ, wherein Het is as hereinbefore defined, R^{m} and Rⁿ are each independently selected from hydrogen, C₁₋₄alkyl and CO₂(CH₂)₀₋₃aryl, and R⁸ is hydrogen or C₁₋₆alkyl,
and wherein R⁷ is optionally substituted by halogen, C₁₋₄alkyl or NR^{o}R^{p}, wherein R^{o} and R^{p} are each independently selected from hydrogen and C₁₋₄alkyl;
and where R⁴ is optionally substituted by hydroxy, fluorine, chlorine, C₁₋₄alkyl, ═O, CO₂H or CO₂C₁₋₄alkyl;
or R³, R⁴ and the nitrogen atom to which they are attached form a heteroaliphatic ring of 4 to 7 ring atoms, where said ring is optionally substituted by halogen, hydroxy, ═O, C₁₋₄alkyl or C₁₋₄alkoxy;
the other of R¹ and R² is hydrogen, fluorine, chlorine, C₁₋₄alkyl, C₂₋₄alkenyl or C₁₋₄alkoxy, where said C₁₋₄alkyl, C₂₋₄alkenyl and C₁₋₄alkoxy groups are optionally substituted by hydroxy or fluorine;
R²⁰ is hydrogen or C₁₋₄alkyl;
L is 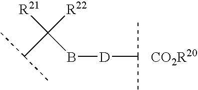
wherein R²¹ and R²² are independently selected from hydrogen, halogen, C₁₋₄alkyl, C₂₋₄alkenyl or C₁₋₄alkoxy;
or R²¹ and R²² are linked to form a C₃₋₈cycloalkyl group;
B is aryl, heteroaryl or CONR²³R²⁴, optionally substituted by halogen, C₁₋₄alkyl, C₂₋₄alkenyl or C₁₋₄alkoxy;
R²³ is hydrogen or C₁₋₆alkyl;
or R²³ is linked to R²¹ and/or R²² to form a 5- to 10-membered ring, where said ring may be saturated, partially saturated or unsaturated, and where said ring is optionally substituted by halogen, C₁₋₄alkyl, C₂₋₄alkenyl, C₂₋₄alkynyl or C₁₋₄alkoxy;
R²⁴ is aryl or heteroaryl;
or R²³, R²⁴ and the nitrogen atom to which they are attached form a 5- to 10-membered mono- or bi-cyclic ring system, where said ring may be saturated, partially saturated or unsaturated, and where said ring is optionally substituted by halogen, C₁₋₄alkyl, C₂₋₄alkenyl, C₂₋₄alkynyl or C₁₋₄alkoxy;
D is a bond, C₁₋₆alkylene, C₂₋₆alkenylene, C₂₋₆alkynylene, aryl or heteroaryl, where said aryl or heteroaryl is optionally substituted by halogen, C₁₋₄alkyl or C₂₋₄alkenyl;
W and Z are independently selected from a bond, C═O, O, S, S(O), S(O)₂, —(CR¹⁰R¹¹)—(CR¹²R¹³)₀₋₁— and NR¹⁰;
X and Y are independently selected from a bond, C═O, O, —CR¹⁴R¹⁵—, —CR¹⁴(OR¹⁵)— and NR¹⁴;
and none, one or two of W, X, Y and Z are a bond;
R¹⁰, R¹¹, R¹², R¹³, R¹⁴ and R¹⁵ are each independently selected from hydrogen, hydroxy, C₁₋₆alkyl, C₂₋₆alkenyl, C₁₋₆alkoxy, C(O)C₁₋₆alkyl, (CH₂)₀₋₃C₃₋₈cycloalkyl, (CH₂)₀₋₃heteroaryl, (CH₂)₀₋₃Het, (CH₂)₀₋₃C(O)(CH₂)₀₋₃Het, (CH₂)₀₋₃NR¹⁶R¹⁷, (CH₂)₀₋₃C(O)(CH₂)₀₋₃NR¹⁶R¹⁷ and NHC(O)(CH₂)₀₋₃NR¹⁶R¹⁷;
R¹⁶ and R¹⁷ are independently selected from hydrogen, C₁₋₆alkyl and (CH₂)₀₋₄NR¹⁸R¹⁹; or R¹⁶, R¹⁷ and the nitrogen atom to which they are attached form a heteroaliphatic ring of 4 to 7 ring atoms, which ring may optionally contain 1 or 2 more heteroatoms selected from O or S or a group S(O), S(O)₂, NH or NC₁₋₄alkyl, and which ring is optionally substituted by halogen, hydroxy, C₁₋₄alkyl or C₁₋₄alkoxy;
R¹⁸ and R¹⁹ are independently selected from hydrogen and C₁₋₆alkyl;
or R¹⁸, R¹⁹ and the nitrogen atom to which they are attached form a heteroaliphatic ring of 4 to 7 ring atoms, which ring may optionally contain 1 or 2 more heteroatoms selected from O or S or a group S(O), S(O)₂, NH or NC₁₋₄alkyl, and which ring is optionally substituted by halogen, hydroxy, C₁₋₄alkyl or C₁₋₄alkoxy;
and pharmaceutically acceptable salts thereof.

2. A compound as claimed in claim 1 of the formula (Io): 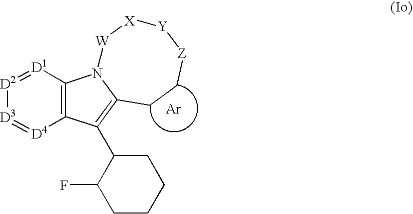
wherein
Ar is a moiety containing at least one aromatic ring and possesses 5-, 6-, 9- or 10-ring atoms optionally containing 1, 2 or 3 heteroatoms independently selected from N, O and S, which ring is optionally substituted at any substitutable position by groups Q¹ and Q²;
Q¹ is halogen, hydroxy, C₁₋₆alkyl, C₁₋₆alkoxy, aryl, heteroaryl, CONR^{c}R^{d}, (CH₂)₀₋₃NR^{c}R^{d}, O(CH₂)₀₋₃C₃₋₈cycloalkyl, O(CH₂)₁₋₃NR^{c}R^{d}, O(CH₂)₀₋₃CONR^{c}R^{d}, O(CH₂)₀₋₃aryl, O(CH₂)₀₋₃heteroaryl, OCHR^{e}R^{f};
R^{c} and R^{d} are each independently selected from hydrogen, C₁₋₄alkyl and C(O)C₁₋₄alkyl;
or R^{c}, R^{d} and the nitrogen atom to which they are attached form a heteroaliphatic ring of 4 to 7 ring atoms, where said ring is optionally substituted by halogen, hydroxy, C₁₋₄alkyl or C₁₋₄alkoxy;
R^{e} and R^{f} are each independently selected from hydrogen and C₁₋₄alkoxy;
or R^{e} and R^{f} are linked by a heteroatom selected from N, O and S to form a heteroaliphatic ring of 4 to 7 ring atoms, where said ring is optionally substituted by halogen, hydroxy, C₁₋₄alkyl or C₁₋₄alkoxy;
and wherein said C₁₋₄alkyl, C₁₋₄alkoxy and aryl groups are optionally substituted by halogen or hydroxy;
Q² is halogen, hydroxy, C₁₋₄alkyl or C₁₋₄alkoxy, where said C₁₋₄alkyl and C₁₋₄alkoxy groups are optionally substituted by halogen or hydroxy;
or Q¹ and Q² may be linked by a bond or a heteroatom selected from N, O and S to form a ring of 4 to 7 atoms, where said ring is optionally substituted by halogen, hydroxy, C₁₋₄alkyl or C₁₋₄alkoxy;
D¹ is N or CR^{a};
D² is Nor CR¹;
D³ is N or CR²;
D⁴ is N or CR^{b};
with the proviso that D² and D³ are not both N;
R^{a} and R^{b} are each independently selected from hydrogen, fluorine, chlorine, C₁₋₄alkyl, C₂₋₄alkenyl or C₁₋₄alkoxy, where said C₁₋₄alkyl, C₂₋₄alkenyl and C₁₋₄alkoxy groups are optionally substituted by hydroxy or fluorine;
one of R¹ or R² is hydrogen, halogen, C₁₋₄alkyl, C₁₋₄alkoxy, CN, CO₂H, CO₂C₁₋₄alkyl, aryl, heteroaryl or C(O)NR³R⁴, where said C₁₋₄alkyl, C₁₋₄alkoxy, aryl and heteroaryl groups are optionally substituted by hydroxy or fluorine;
R³ is hydrogen or C₁₋₄alkyl;
R⁴ is hydrogen, C₁₋₄alkyl, C₂₋₄alkenyl, (CH₂)₀₋₃R⁵ or SO₂R⁶;
R⁵ and R⁶ are as defined in claim 1;
and where R⁴ is optionally substituted by hydroxy, fluorine, chlorine, C₁₋₄alkyl, ═O, CO₂H or CO₂C₁₋₄alkyl;
or R³, R⁴ and the nitrogen atom to which they are attached form a heteroaliphatic ring of 4 to 7 ring atoms, where said ring is optionally substituted by halogen, hydroxy, ═O, C₁₋₄alkyl or C₁₋₄alkoxy;
the other of R¹ and R² is hydrogen, fluorine, chlorine, C₁₋₄alkyl, C₁₋₄alkenyl or C₁₋₄alkoxy, where said C₁₋₄alkyl, C₂₋₄alkenyl and C₁₋₄alkoxy groups are optionally substituted by hydroxy or fluorine;
W, X, Y and Z are as defined in claim 1;
and pharmaceutically acceptable salts thereof.

3. A compound as claimed in claim 1 wherein Ar is a five- or six-membered aromatic ring optionally containing 1, 2 or 3 heteroatoms independently selected from N, O and S, and which ring is optionally substituted by groups Q¹ and Q² as defined in claim 1.

4. A compound as claimed in claim 1 wherein D¹ is CR^{a} where R^{a} is as defined in claim 1.

5. A compound as claimed in claim 1 wherein D⁴ is CR^{b} where R^{b} is as defined in claim 1.

6. A compound as claimed in claim 1 wherein D² is CR¹ where R¹ is as defined in claim 1.

7. A compound as claimed in wherein D³ is CR² where R² is as defined in claim 1.

8. A compound as claimed in claim 1 wherein W is a bond, C═O, —(CR¹⁰R¹¹)—(CR¹²R¹³)₀₋₁— or NR¹⁰ where R¹⁰, R¹¹, R¹² and R¹³ are as defined in claim 1.

9. A compound as claimed in claim 1 wherein Z is a bond, C═O, O, —(CR¹⁰R¹¹)—(CR¹²R¹³)₀₋₁— or NR¹⁰ where R¹⁰, R¹¹, R¹² and R¹³ are as defined in claim 1.

10. A compound as claimed in claim 1 wherein X is C═O, —CR¹⁴R¹⁵— or —CR¹⁴(OR¹⁵)— where R¹⁴ and R¹⁵ are as defined in claim 1.

11. A compound as claimed in claim 1 wherein Y is —CR¹⁴R¹⁵— or NR¹⁴ where R¹⁴ and R¹⁵ are as defined in claim 1.

12. A compound as claimed in claim 1 that has the following relative stereochemical configuration:

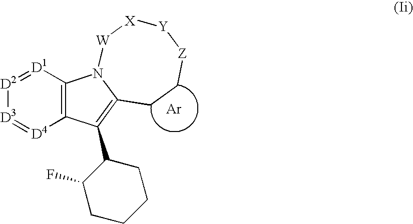

13. A compound as claimed in claim 1 of formula (Ia) and pharmaceutically acceptable salts thereof: 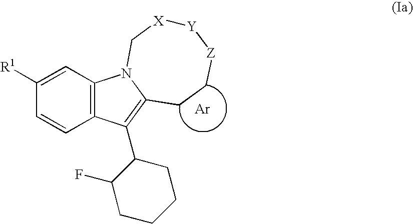
wherein Ar, R¹, X, Y and Z are as defined in claim 1.

14. A compound as claimed in claim 13 of formula (Ia) that has the following relative stereochemical configuration:

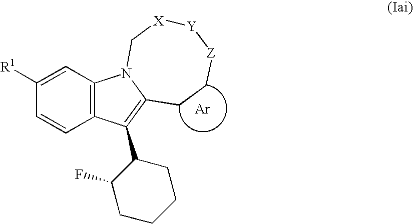

15. A compound as claimed in claim 1 selected from:
14-[(1R,2S) or (1S,2R)-2-fluorocyclohexyl]-6-isopropyl-3-(pyridin-2-ylmethoxy)-5,6,7,8-tetrahydroindolo[2,1-a][2,5]benzodiazocine-11-carboxylic acid;
7-[[2-(dimethylamino)ethyl](methyl)amino]-14-[(1S,2S) or (1S,2R)-2-fluorocyclohexyl]-7,8-dihydro-6H-indolo[1,2-e][1,5]benzoxazocine-11-carboxylic acid;
14-[(1R,2S) or (1S,2R)-2-fluorocyclohexyl]-6-isopropyl-3-(pyridin-2-yloxy)-5,6,7,8-tetrahydroindolo[2,1-a][2,5]benzodiazocine-11-carboxylic acid;
6-[2-(dimethylamino)ethyl]-14-[(1S,2R)-2-fluorocyclohexyl]-3-methoxy-5,6,7,8-tetrahydroindolo[2,1-a][2,5]benzodiazocine-11-carboxylic acid; and
6-[2-(dimethylamino)ethyl]-14-[(1R,2S)-2-fluorocyclohexyl]-3-methoxy-5,6,7,8-tetrahydroindolo[2,1-a][2,5]benzodiazocine-11-carboxylic acid;
6-[2-(dimethylaminonio)ethyl]-14-[trans-2-fluorocyclohexyl]-11-(1H-tetrazol-5-yl)-5,6,7,8-tetrahydroindolo[2,1-a][2,5]benzodiazocin-6-ium bis(trifluoroacetate);
6-ethyl-14-[(1R,2S) or (1S,2R)-2-fluorocyclohexyl]-3-(pyridin-2-ylmethoxy)-5,6,7,8-tetrahydroindolo[2,1-a][2,5]benzodiazocine-11-carboxylic acid;
14-[(1R,2S) or (1S,2R)-2-fluorocyclohexyl]-6-isopropyl-3-(pyridin-3-ylmethoxy)-5,6,7,8-tetrahydroindolo[2,1-a][2,5]benzodiazocine-11-carboxylic acid;
14-[(1R,2S)-2-fluorocyclohexyl]-6-isopropyl-3-(pyridazin-3-ylmethoxy)-5,6,7,8-tetrahydroindolo[2,1-a][2,5]benzodiazocine-11-carboxylic acid;
(7R)-7-(dimethylamino)-14-[(1R,2S) or (1S,2R)-2-fluorocyclohexyl]-3-(pyridin-3-ylmethoxy)-7,8-dihydro-6H-indolo[1,2-e][1,5]benzoxazocine-11-carboxylic acid;
(7R and 7S)-7-[[2-(dimethylamino)ethyl](methyl)amino]-14-[(1S,2R) or (1R,2S)-2-fluorocyclohexyl]-3-methoxy-7,8-dihydro-6H-indolo[1,2-e][1,5]benzoxazocine-11-carboxylic acid;
(2E)-3-{4-[({1-[({14-[(trans)-2-fluorocyclohexyl]-6-isopropyl-3-methoxy-5,6,7,8-tetrahydroindolo[2,1-a][2,5]benzodiazocin-11-yl}carbonyl)amino]cyclopentyl}carbonyl)amino]phenyl}acrylic acid;
7R and 7S-[2-(dimethylamino)ethyl]-14-[(1S,2R) or (1R,2S)-2-fluorocyclohexyl]-3-(pyridin-3-ylmethoxy)-7,8-dihydro-6H-indolo[1,2-e][1,5]benzoxazocine-11-carboxylic acid;
7R and 7S-[2-(dimethylamino)ethyl]-14-[(1R,2S) or (1S,2R)-2-fluorocyclohexyl]-3-methoxy-7,8-dihydro-6H-indolo[1,2-e][1,5]benzoxazocine-11-carboxylic acid;
7R and 7S-[(dimethylamino)methyl]-14-[(1R,2S) or (1S,2R)-2-fluorocyclohexyl]-3-(pyridazin-3-ylmethoxy)-7,8-dihydro-6H-indolo[1,2-e][1,5]benzoxazocine-11-carboxylic acid;
6-[2-(dimethylamino)ethyl]-14-[trans-2-fluorocyclohexyl]-7-oxo-5,6,7,8-tetrahydroindolo[2,1-a][2,5]benzodiazocine-11-carboxylic acid;
14-[trans-2-fluorocyclohexyl]-6-methyl-5,6,7,8-tetrahydroindolo[2,1-a][2,5]benzodiazocine-11-carboxylic acid;
6-[2-(dimethylamino)ethyl]-14-[trans-2-fluorocyclohexyl]-3-methoxy-7-oxo-5,6,7,8-tetrahydroindolo[2,1-a][2,5]benzodiazocine-11-carboxylic acid;
14-[trans-2-fluorocyclohexyl]-3-methoxy-6-methyl-5,6,7,8-tetrahydroindolo[2,1-a][2,5]benzodiazocine-11-carboxylic acid;
5-[trans-2-(dimethylamino)ethyl]-13-[(trans-2-fluorocyclohexyl]-6,7-dihydro-5H-indolo[1,2-d][1,4]benzodiazepine-10-carboxylic acid;
5-[2-(dimethylamino)-2-oxoethyl]-13-[trans-2-fluorocyclohexyl]-6-oxo-6,7-dihydro-5H-indolo[1,2-d][1,4]benzodiazepine-10-carboxylic acid;
6-[2-(dimethylamino)ethyl]-14-[trans-2-fluorocyclohexyl]-3-methyl-5,6,7,8-tetrahydroindolo[2,1-a][2,5]benzodiazocine-11-carboxylic acid;
3-(benzyloxy)-14-[trans-2-fluorocyclohexyl]-6-methyl-5,6,7,8-tetrahydroindolo[2,1-a][2,5]benzodiazocine-11-carboxylic acid;
3-(benzyloxy)-6-[2-(dimethylamino)ethyl]-14-[trans-2-fluorocyclohexyl]-5,6,7,8-tetrahydroindolo[2,1-a][2,5]benzodiazocine-11-carboxylic acid;
6-[2-(dimethylamino)ethyl]-14-[trans-2-fluorocyclohexyl]-N,3-dimethyl-5,6,7,8-tetrahydroindolo[2,1-a][2,5]benzodiazocine-11-carboxamide;
6-(N,N-dimethylglycyl)-14-[trans-2-fluorocyclohexyl]-3-methoxy-5,6,7,8-tetrahydroindolo[2,1-a][2,5]benzodiazocine-11-carboxylic acid;
6-(N,N-dimethylglycyl)-14-[trans-2-fluorocyclohexyl]-3-methoxy-5,6,7,8-tetrahydroindolo[2,1-a][2,5]benzodiazocine-11-carboxylic acid;
3-chloro-6-[2-(dimethylamino)ethyl]-14-[trans-2-fluorocyclohexyl]-5,6,7,8-tetrahydroindolo[2,1-a][2,5]benzodiazocine-11-carboxylic acid;
6-(N,N-dimethylglycyl)-14-[trans-2-fluorocyclohexyl]-5,6,7,8-tetrahydroindolo[2,1-a][2,5]benzodiazocine-11-carboxylic acid;
(7S)-7-[2-(dimethylamino)ethoxy]-14-[trans-2-fluorocyclohexyl]-7,8-dihydro-6H-indolo[1,2-e][1,5]benzoxazocine-11-carboxylic acid;
(7R)-7-[2-(dimethylamino)ethoxy]-14-[trans-2-fluorocyclohexyl]-7,8-dihydro-6H-indolo[1,2-e][1,5]benzoxazocine-11-carboxylic acid;
3-(benzyloxy)-6-[2-(dimethylamino)ethyl]-N-(ethylsulfonyl)-14-[trans-2-fluorocyclohexyl]-5,6,7,8-tetrahydroindolo[2,1-a][2,5]benzodiazocine-11-carboxamide;
3-(benzyloxy)-6-[2-(dimethylamino)ethyl]-N-(ethylsulfonyl)-14-[trans-2-fluorocyclohexyl]-5,6,7,8-tetrahydroindolo[2,1-a][2,5]benzodiazocine-11-carboxamide;
6-[2-(dimethylamino)ethyl]-14-[trans-2-fluorocyclohexyl]-3-(pyridin-3-ylmethoxy)-5,6,7,8-tetrahydroindolo[2,1-a][2,5]benzodiazocine-11-carboxylic acid;
3-[2-(dimethylamino)ethoxy]-14-[trans-2-fluorocyclohexyl]-6-methyl-5,6,7,8-tetrahydroindolo[2,1-a][2,5]benzodiazocine-11-carboxylic acid;
6-[2-(dimethylamino)ethyl]-14-[trans-2-fluorocyclohexyl]-3-[(1-methyl-1H-1,2,4-triazol-3-yl)methoxy]-5,6,7,8-tetrahydroindolo[2,1-a][2,5]benzodiazocine-11-carboxylic acid;
6-[2-(dimethylamino)ethyl]-14-[trans-2-fluorocyclohexyl]-3-[(1-methyl-1H-1,2,4-triazol-3-yl)methoxy]-5,6,7,8-tetrahydroindolo[2,1-a][2,5]benzodiazocine-11-carboxylic acid;
3-fluoro-13-[trans-2-fluorocyclohexyl]-6-oxo-6,7-dihydro-5H-indolo[1,2-d][1,4]benzodiazepine-10-carboxylic acid;
3-fluoro-13-[trans-2-fluorocyclohexyl]-6-oxo-6,7-dihydro-5H-indolo[1,2-d][1,4]benzodiazepine-10-carboxylic acid;
3-fluoro-13-[trans-2-fluorocyclohexyl]-6-oxo-6,7-dihydro-5H-indolo[1,2-d][1,4]benzodiazepine-10-carboxylic acid;
3-fluoro-13-[trans-2-fluorocyclohexyl]-6,7-dihydro-5H-indolo[1,2-d][1,4]benzodiazepine-10-carboxylic acid;
6-(N,N-dimethylglycyl)-14-[trans-2-fluorocyclohexyl]-3-(pyridin-3-ylmethoxy)-5,6,7,8-tetrahydroindolo[2,1-a][2,5]benzodiazocine-11-carboxylic acid;
6-(N,N-dimethylglycyl)-14-[trans-2-fluorocyclohexyl]-3-(pyridin-2-ylmethoxy)-5,6,7,8-tetrahydroindolo[2,1-a][2,5]benzodiazocine-11-carboxylic acid;
6-(N,N-dimethylglycyl)-14-[trans-2-fluorocyclohexyl]-3-(pyridin-4-ylmethoxy)-5,6,7,8-tetrahydroindolo[2,1-a][2,5]benzodiazocine-11-carboxylic acid;
6-(N,N-dimethylglycyl)-3-ethoxy-14-[trans-2-fluorocyclohexyl]-5,6,7,8-tetrahydroindolo[2,1-a][2,5]benzodiazocine-11-carboxylic acid;
3-ethoxy-14-[trans-2-fluorocyclohexyl]-6-methyl-5,6,7,8-tetrahydroindolo[2,1-a][2,5]benzodiazocine-11-carboxylic acid;
6-[2-(dimethylamino)ethyl]-3-ethoxy-14-[trans-2-fluorocyclohexyl]-5,6,7,8-tetrahydroindolo[2,1-a][2,5]benzodiazocine-11-carboxylic acid;
14-[trans-2-fluorocyclohexyl]-6-isopropyl-3-propoxy-5,6,7,8-tetrahydroindolo[2,1-a][2,5]benzodiazocine-11-carboxylic acid;
6-[2-(dimethylamino)ethyl]-14-[trans-2-fluorocyclohexyl]-3-propoxy-5,6,7,8-tetrahydroindolo[2,1-a][2,5]benzodiazocine-11-carboxylic acid;
14-[trans-2-fluorocyclohexyl]-3-propoxy-6-(2-pyrrolidin-1-ylethyl)-5,6,7,8-tetrahydroindolo[2,1-a][2,5]benzodiazocine-11-carboxylic acid;
14-[trans-2-fluorocyclohexyl]-6-(2-morpholin-4-ylethyl)-3-propoxy-5,6,7,8-tetrahydroindolo[2,1-a][2,5]benzodiazocine-11-carboxylic acid;
14-[trans-fluorocyclohexyl]-6-methyl-3-(pyridin-2-ylmethoxy)-5,6,7,8-tetrahydroindolo[2,1-a][2,5]benzodiazocine-11-carboxylic acid;
6-[2-(diethylamino)ethyl]-14-[trans-2-fluorocyclohexyl]-3-(pyridin-2-ylmethoxy)-5,6,7,8-tetrahydroindolo[2,1-a][2,5]benzodiazocine-11-carboxylic acid;
14-[trans-2-fluorocyclohexyl]-3-(pyridin-2-ylmethoxy)-6-(2-pyrrolidin-1-ylethyl)-5,6,7,8-tetrahydroindolo[2,1-a][2,5]benzodiazocine-11-carboxylic acid;
14-[trans-2-fluorocyclohexyl]-6-methyl-3-(pyridin-3-ylmethoxy)-5,6,7,8-tetrahydroindolo[2,1-a][2,5]benzodiazocine-11-carboxylic acid;
14-[trans-2-fluorocyclohexyl]-6-isopropyl-3-(pyridin-3-ylmethoxy)-5,6,7,8-tetrahydroindolo[2,1-a][2,5]benzodiazocine-11-carboxylic acid;
14-[trans-2-fluorocyclohexyl]-6-methyl-3-(pyridin-4-ylmethoxy)-5,6,7,8-tetrahydroindolo[2,1-a][2,5]benzodiazocine-11-carboxylic acid;
6-[2-(dimethylamino)ethyl]-14-[trans-2-fluorocyclohexyl]-3-(pyridin-4-ylmethoxy)-5,6,7,8-tetrahydroindolo[2,1-a][2,5]benzodiazocine-11-carboxylic acid;
14-[trans-2-fluorocyclohexyl]-6-(2-morpholin-4-ylethyl)-3-(pyridin-4-ylmethoxy)-5,6,7,8-tetrahydroindolo[2,1-a][2,5]benzodiazocine-11-carboxylic acid;
3-ethoxy-14-[trans-2-fluorocyclohexyl]-6-isopropyl-5,6,7,8-tetrahydroindolo[2,1-a][2,5]benzodiazocine-11-carboxylic acid;
3-ethoxy-14-[trans-2-fluorocyclohexyl]-6-(2-pyrrolidin-1-ylethyl)-5,6,7,8-tetrahydroindolo[2,1-a][2,5]benzodiazocine-11-carboxylic acid;
3-ethoxy-14-[trans-2-fluorocyclohexyl]-6-(2-morpholin-4-ylethyl)-5,6,7,8-tetrahydroindolo[2,1-a][2,5]benzodiazocine-11-carboxylic acid;
14-[trans-2-fluorocyclohexyl]-6-methyl-3-propoxy-5,6,7,8-tetrahydroindolo[2,1-a][2,5]benzodiazocine-11-carboxylic acid;
3-(cyclopropylmethoxy)-6-[2-(dimethylamino)ethyl]-14-[trans-2-fluorocyclohexyl]-5,6,7,8-tetrahydroindolo[2,1-a][2,5]benzodiazocine-11-carboxylic acid;
3-(cyclopropylmethoxy)-14-[trans-2-fluorocyclohexyl]-6-(2-pyrrolidin-1-ylethyl)-5,6,7,8-tetrahydroindolo[2,1-a][2,5]benzodiazocine-11-carboxylic acid;
3-(cyclopropylmethoxy)-14-[trans-2-fluorocyclohexyl]-6-(2-morpholin-4-ylethyl)-5,6,7,8-tetrahydroindolo[2,1-a][2,5]benzodiazocine-11-carboxylic acid;
14-[trans-2-fluorocyclohexyl]-6-(2-morpholin-4-ylethyl)-3-(pyridin-2-ylmethoxy)-5,6,7,8-tetrahydroindolo[2,1-a][2,5]benzodiazocine-11-carboxylic acid;
6-[2-(diethylamino)ethyl]-14-[trans-2-fluorocyclohexyl]-3-(pyridin-3-ylmethoxy)-5,6,7,8-tetrahydroindolo[2,1-a][2,5]benzodiazocine-11-carboxylic acid;
14-[trans-2-fluorocyclohexyl]-6-(2-morpholin-4-ylethyl)-3-(pyridin-3-ylmethoxy)-5,6,7,8-tetrahydroindolo[2,1-a][2,5]benzodiazocine-11-carboxylic acid;
6-[2-(diethylamino)ethyl]-14-[trans-2-fluorocyclohexyl]-3-(pyridin-4-ylmethoxy)-5,6,7,8-tetrahydroindolo[2,1-a][2,5]benzodiazocine-11-carboxylic acid;
14-[trans-2-fluorocyclohexyl]-3-(pyridin-4-ylmethoxy)-6-(2-pyrrolidin-1-ylethyl)-5,6,7,8-tetrahydroindolo[2,1-a][2,5]benzodiazocine-11-carboxylic acid;
14-[trans-2-fluorocyclohexyl]-6-isopropyl-5,6,7,8-tetrahydroindolo[2,1-a][2,5]benzodiazocine-11-carboxylic acid;
14-[trans-2-fluorocyclohexyl]-6-(2-pyrrolidin-1-ylethyl)-5,6,7,8-tetrahydroindolo[2,1-a][2,5]benzodiazocine-11-carboxylic acid;
14-[trans-2-fluorocyclohexyl]-6-(2-morpholin-4-ylethyl)-5,6,7,8-tetrahydroindolo[2,1-a][2,5]benzodiazocine-11-carboxylic acid;
14-[trans-2-fluorocyclohexyl]-6-isopropyl-3-methoxy-5,6,7,8-tetrahydroindolo[2,1-<z][2,5]benzodiazocine-11-carboxylic acid;
6-[2-(diethylamino)ethyl]-14-[trans-2-fluorocyclohexyl]-3-methoxy-5,6,7,8-tetrahydroindolo[2,1-a][2,5]benzodiazocine-11-carboxylic acid;
14-[trans-2-fluorocyclohexyl]-3-methoxy-6-(2-pyrrolidin-1-ylethyl)-5,6,7,8-tetrahydroindolo[2,1-a][2,5]benzodiazocine-11-carboxylic acid;
14-[trans-2-fluorocyclohexyl]-3-methoxy-6-(2-morpholin-4-ylethyl)-5,6,7,8-tetrahydroindolo[2,1-a][2,5]benzodiazocine-11-carboxylic acid;
3-fluoro-14-[trans-2-fluorocyclohexyl]-6-methyl-5,6,7,8-tetrahydroindolo[2,1-a][2,5]benzodiazocine-11-carboxylic acid;
3-fluoro-14-[trans-2-fluorocyclohexyl]-6-isopropyl-5,6,7,8-tetrahydroindolo[2,1-a][2,5]benzodiazocine-11-carboxylic acid;
6-[2-(dimethylamino)ethyl]-3-fluoro-14-[trans-2-fluorocyclohexyl]-5,6,7,8-tetrahydroindolo[2,1-a][2,5]benzodiazocine-11-carboxylic acid;
6-[2-(diethylamino)ethyl]-3-fluoro-14-[trans-2-fluorocyclohexyl]-5,6,7,8-tetrahydroindolo[2,1-a][2,5]benzodiazocine-11-carboxylic acid;
3-fluoro-14-[trans-2-fluorocyclohexyl]-6-(2-pyrrolidin-1-ylethyl)-5,6,7,8-tetrahydroindolo[2,1-a][2,5]benzodiazocine-11-carboxylic acid;
3-fluoro-14-[trans-2-fluorocyclohexyl]-6-(2-morpholin-4-ylethyl)-5,6,7,8-tetrahydroindolo[2,1-a][2,5]benzodiazocine-11-carboxylic acid;
3-chloro-6-[2-(diethylamino)ethyl]-14-[trans-2-fluorocyclohexyl]-5,6,7,8-tetrahydroindolo[2,1-a][2,5]benzodiazocine-11-carboxylic acid;
3-chloro-14-[trans-2-fluorocyclohexyl]-6-(2-pyrrolidin-1-ylethyl)-5,6,7,8-tetrahydroindolo[2,1-a][2,5]benzodiazocine-11-carboxylic acid;
3-chloro-14-[trans-2-fluorocyclohexyl]-6-(2-morpholin-4-ylethyl)-5,6,7,8-tetrahydroindolo[2,1-a][2,5]benzodiazocine-11-carboxylic acid;
2-fluoro-14-[trans-2-fluorocyclohexyl]-6-isopropyl-5,6,7,8-tetrahydroindolo[2,1-a][2,5]benzodiazocine-11-carboxylic acid;
6-[2-(dimethylamino)ethyl]-2-fluoro-14-[trans-2-fluorocyclohexyl]-5,6,7,8-tetrahydroindolo[2,1-a][2,5]benzodiazocine-11-carboxylic acid;
6-[2-(diethylamino)ethyl]-2-fluoro-14-[trans-2-fluorocyclohexyl]-5,6,7,8-tetrahydroindolo[2,1-a][2,5]benzodiazocine-11-carboxylic acid;
2-fluoro-14-[trans-2-fluorocyclohexyl]-6-(2-pyrrolidin-1-ylethyl)-5,6,7,8-tetrahydroindolo[2,1-a][2,5]benzodiazocine-11-carboxylic acid;
2-fluoro-14-[trans-2-fluorocyclohexyl]-6-(2-morpholin-4-ylethyl)-5,6,7,8-tetrahydroindolo[2,1-a][2,5]benzodiazocine-11-carboxylic acid;
3-(benzyloxy)-14-[trans-2-fluorocyclohexyl]-6-isopropyl-5,6,7,8-tetrahydroindolo[2,1-a][2,5]benzodiazocine-11-carboxylic acid;
3-(benzyloxy)-6-[2-(diethylamino)ethyl]-14-[trans-2-fluorocyclohexyl]-5,6,7,8-tetrahydroindolo[2,1-a][2,5]benzodiazocine-11-carboxylic acid;
3-(benzyloxy)-14-[trans-2-fluorocyclohexyl]-6-(2-pyrrolidin-1-ylethyl)-5,6,7,8-tetrahydroindolo[2,1-a][2,5]benzodiazocine-11-carboxylic acid;
6-[2-(dimethylamino)ethyl]-4-fluoro-14-[trans-2-fluorocyclohexyl]-5,6,7,8-tetrahydroindolo[2,1-a][2,5]benzodiazocine-11-carboxylic acid;
6-[2-(diethylamino)ethyl]-4-fluoro-14-[trans-2-fluorocyclohexyl]-5,6,7,8-tetrahydroindolo[2,1-a][2,5]benzodiazocine-11-carboxylic acid;
4-fluoro-14-[trans-2-fluorocyclohexyl]-6-(2-pyrrolidin-1-ylethyl)-5,6,7,8-tetrahydroindolo[2,1-a][2,5]benzodiazocine-11-carboxylic acid;
4-fluoro-14-[trans-2-fluorocyclohexyl]-6-(2-morpholin-4-ylethyl)-5,6,7,8-tetrahydroindolo[2,1-a][2,5]benzodiazocine-11-carboxylic acid;
6-[2-(dimethylamino)ethyl]-14-[trans-2-fluorocyclohexyl]-7-oxo-3-(pyridin-2-ylmethoxy)-5,6,7,8-tetrahydroindolo[2,1-a][2,5]benzodiazocine-11-carboxylic acid;
3-chloro-6-[2-(dimethylamino)ethyl]-14-[trans-2-fluorocyclohexyl]-7-oxo-5,6,7,8-tetrahydroindolo[2,1-a][2,5]benzodiazocine-11-carboxylic acid;
6-[2-(dimethylamino)ethyl]-2-fluoro-14-[trans-2-fluorocyclohexyl]-7-oxo-5,6,7,8-tetrahydroindolo[2,1-a][2,5]benzodiazocine-11-carboxylic acid;
14-[trans-2-fluorocyclohexyl]-3-methoxy-6-(pyridin-4-ylmethyl)-5,6,7,8-tetrahydroindolo[2,1-a][2,5]benzodiazocine-11-carboxylic acid;
14-[trans-2-fluorocyclohexyl]-3-methoxy-6-(pyridin-3-ylmethyl)-5,6,7,8-tetrahydroindolo[2,1-a][2,5]benzodiazocine-11-carboxylic acid;
6-[2-(dimethylamino)ethyl]-14-[(1R,2S or 1S,2R))-2-fluorocyclohexyl]-3-(pyridin-2-yloxy)-5,6,7,8-tetrahydroindolo[2,1-a][2,5]benzodiazocine-11-carboxylic acid;
3-chloro-6-(N,N-dimethylglycyl)-14-[trans-2-fluorocyclohexyl]-5,6,7,8-tetrahydroindolo[2,1-a][2,5]benzodiazocine-11-carboxylic acid;
6-[2-(dimethylamino)ethyl]-14-[trans-2-fluorocyclohexyl]-3-(pyridin-2-ylmethoxy)-5,6,7,8-tetrahydroindolo[2,1-a][2,5]benzodiazocine-11-carboxylic acid;
6-[2-(diethylamino)ethyl]-3-ethoxy-14-[trans-2-fluorocyclohexyl]-5,6,7,8-tetrahydroindolo[2,1-a][2,5]benzodiazocine-11-carboxylic acid;
5-[2-(dimethylamino)ethyl]-13-[trans-2-fluorocyclohexyl]-3-methoxy-6,7-dihydro-5H-indolo[1,2-d][1,4]benzodiazepine-10-carboxylic acid;
13-[trans-2-fluorocyclohexyl]-3-methoxy-5-methyl-6,7-dihydro-5H-indolo[1,2-d][1,4]benzodiazepine-10-carboxylic acid;
14-[trans-2-fluorocyclohexyl]-3-methoxy-6-(1-methyl-L-prolyl)-5,6,7,8-tetrahydroindolo[2,1-a][2,5]benzodiazocine-11-carboxylic acid;
6-[2-(dimethylamino)ethyl]-14-[trans-2-fluorocyclohexyl]-3-(pyrimidin-2-yloxy)-5,6,7,8-tetrahydroindolo[2,1-a][2,5]benzodiazocine-11-carboxylic acid;
5-[2-(dimethylamino)-2-oxoethyl]-13-[trans-2-fluorocyclohexyl]-3-methoxy-6-oxo-6,7-dihydro-5H-indolo[1,2-d][1,4]benzodiazepine-10-carboxylic acid;
(7R)-7-(dimethylamino)-14-[(1R,2R)-2-fluorocyclohexyl]-7,8-dihydro-6H-indolo[1,2-e][1,5]benzoxazocine-11-carboxylic acid;
14-[trans-2-fluorocyclohexyl]-6-isopropyl-3-(pyridin-2-yloxy)-5,6,7,8-tetrahydroindolo[2,1-a][2,5]benzodiazocine-11-carboxylic acid;
6-ethyl-14-[(1R,2S or 1S,2R)-2-fluorocyclohexyl]-3-(pyridazin-3-ylmethoxy)-5,6,7,8-tetrahydroindolo[2,1-a][2,5]benzodiazocine-11-carboxylic acid;
6-cyclopropyl-14-[(1R,2S or 1S,2R)-2-fluorocyclohexyl]-3-(pyridazin-3-ylmethoxy)-5,6,7,8-tetrahydroindolo[2,1-a][2,5]benzodiazocine-11-carboxylic acid;
14-[(1R,2S or 1S,2R)-2-fluorocyclohexyl]-6-isopropyl-3-(pyridazin-4-ylmethoxy)-5,6,7,8-tetrahydroindolo[2,1-a][2,5]benzodiazocine-11-carboxylic acid;
14-[(trans-2-fluorocyclohexyl]-6-isopropyl-3-[(1-oxidopyridin-2-yl)oxy]-5,6,7,8-tetrahydroindolo[2,1-a][2,5]benzodiazocine-11-carboxylic acid;
3-(benzyloxy)-5-[2-(dimethylamino)ethyl]-13-[trans-2-fluorocyclohexyl]-6,7-dihydro-5H-indolo[1,2-d][1,4]benzodiazepine-10-carboxylic acid;
6-ethyl-14-[(1R,2S or 1S,2R)-2-fluorocyclohexyl]-3-(pyridin-3-ylmethoxy)-5,6,7,8-tetrahydroindolo[2,1-a][2,5]benzodiazocine-11-carboxylic acid;
6-ethyl-14-[(1R,2S, or 1S,2R)-2-fluorocyclohexyl]-(pyridin-4-ylmethoxy)-5,6,7,8-tetrahydroindolo[2,1-a][2,5]benzodiazocine-11-carboxylic acid;
6-ethyl-14-[(1R,2S or 1S,2R)-2-fluorocyclohexyl]-3-(pyrimidin-2-ylmethoxy)-5,6,7,8-tetrahydroindolo[2,1-a][2,5]benzodiazocine-11-carboxylic acid;
14-[(1R,2S) or (1S,2R)-2-fluorocyclohexyl]-6-isopropyl-3-(pyridin-4-ylmethoxy)-5,6,7,8-tetrahydroindolo[2,1-a][2,5]benzodiazocine-11-carboxylic acid;
14-[(1R,2S or 1S,2R)-2-fluorocyclohexyl]-6-isopropyl-3-(pyrazin-4-yloxy)-5,6,7,8-tetrahydroindolo[2,1-a][2,5]benzodiazocine-11-carboxylic acid;
6-ethyl-14-[(1R,2S or 1S,2R)-2-fluorocyclohexyl]-3-(pyrazin-2-ylmethoxy)-5,6,7,8-tetrahydroindolo[2,1-a][2,5]benzodiazocine-11-carboxylic acid;
14-[(1R,2S or 1S,2R))-fluorocyclohexyl]-6-propyl-3-(1,3-thiazol-2-yloxy)-5,6,7,8-tetrahydroindolo[2,1-a][2,5]benzodiazocine-11-carboxylic acid;
(7R)-7-(dimethylamino)-14-[(1R,2S) or (1S,2R)-2-fluorocyclohexyl]-3-(pyridin-2-ylmethoxy)-7,8-dihydro-6H-indolo[1,2-e][1,5]benzoxazocine-11-carboxylic acid;
indolo[2,1-a][2,5]benzodiazocine-11-carboxylic acid, 14-[(1R,2S) or (1S,2R)-2-fluorocyclohexyl]-5,6,7,8-tetrahydro-3-[(6-methoxy-3-pyridazinyl)methoxy]-6-(1-methylethyl)-;
14-[(1R,2S) or (1S,2R)-2-fluorocyclohexyl]-6-isopropyl-3-(pyrimidin-4-ylmethoxy)-5,6,7,8-tetrahydroindolo[2,1-a][2,5]benzodiazocine-11-carboxylic acid;
4-[(1R,2S) or (1S,2R)-2-fluorocyclohexyl]-3-(pyrimidin-4-ylmethoxy)-6-(tetrahydrofuran-3-yl)-5,6,7,8-tetrahydroindolo[2,1-a][2,5]benzodiazocine-11-carboxylic acid;
14-[(1R,2S) or (1S,2R)-2-fluorocyclohexyl]-3-(pyridin-3-ylmethoxy)-6-(tetrahydrofuran-3-yl)-5,6,7,8-tetrahydroindolo[2,1-a][2,5]benzodiazocine-11-carboxylic acid;
7-{[[2-(dimethylamino)ethyl](methyl)amino]methyl}-14-[(1R,2S) or (1S,2R)-2-fluorocyclohexyl]-3-(pyridin-3-ylmethoxy)-7,8-dihydro-6H-indolo[1,2-e][1,5]benzoxazocine-11-carboxylic acid;
7-{[[2-(dimethylamino)ethyl](methyl)amino]methyl}-14-[(1R,2S) or (1S,2R)-2-fluorocyclohexyl]-3-(pyridin-2-ylmethoxy)-7,8-dihydro-6H-indolo[1,2-e][1,5]benzoxazocine-11-carboxylic acid;
7-{[[2-(dimethylamino)ethyl](methyl)amino]methyl}-14-[(1R,2S) or (1S,2R)-2-fluorocyclohexyl]-3-(pyridazin-3-ylmethoxy)-7,8-dihydro-6H-indolo[1,2-e][1,5]benzoxazocine-11-carboxylic acid;
7-{[[2-(dimethylamino)ethyl](methyl)amino]methyl}-14-[(1R,2S) or (1S,2R)-2-fluorocyclohexyl]-3-(1-pyridin-3-ylethoxy)-7,8-dihydro-6H-indolo[1,2-e][1,5]benzoxazocine-11-carboxylic acid;
7-{[[2-(dimethylamino)ethyl](methyl)amino]methyl}-14-[(1R,2S) or (1S,2R)-2-fluorocyclohexyl]-3-(1-pyridin-2-ylethoxy)-7,8-dihydro-6H-indolo[1,2-e][1,5]benzoxazocine-11-carboxylic acid;
7-{[[2-(dimethylamino)ethyl](methyl)amino]methyl}-14-[(1R,2S) or (1S,2R)-2-fluorocyclohexyl]-3-(1-pyridazin-3-ylethoxy)-7,8-dihydro-6H-indolo[1,2-e][1,5]benzoxazocine-11-carboxylic acid;
7-{[[2-(dimethylamino)ethyl](methyl)amino]methyl}-14-[(1R,2S) or (1S,2R)-2-fluorocyclohexyl]-3-(pyridin-2-yloxy)-7,8-dihydro-6H-indolo[1,2-e][1,5]benzoxazocine-11-carboxylic acid;
7-{[[2-(dimethylamino)ethyl](methyl)amino]methyl}-14-[(1R,2S) or (1S,2R)-2-fluorocyclohexyl]-3-methoxy-7,8-dihydro-6H-indolo[1,2-e][1,5]benzoxazocine-11-carboxylic acid;
7-[(dimethylamino)methyl]-14-[(1R,2S) or (1S,2R)-2-fluorocyclohexyl]-3-(pyridin-5-ylmethoxy)-7,8-dihydro-6H-indolo[1,2-e][1,5]benzoxazocine-11-carboxylic acid;
7-[(dimethylamino)methyl]-14-[(1R,2S) or (1S,2R)-2-fluorocyclohexyl]-3-(pyridin-2-ylmethoxy)-7,8-dihydro-6H-indolo[1,2-e][1,5]benzoxazocine-11-carboxylic acid;
7-[(dimethylamino)methyl]-14-[(1R,2S) or (1S,2R)-2-fluorocyclohexyl]-3-(1-pyridin-3-ylethoxy)-7,8-dihydro-6H-indolo[1,2-e][1,5]benzoxazocine-11-carboxylic acid;
7-[(dimethylamino)methyl]-14-[(1R,2S) or (1S,2R)-2-fluorocyclohexyl]-3-(1-pyridin-2-ylethoxy)-7,8-dihydro-6H-indolo[1,2-e][1,5]benzoxazocine-11-carboxylic acid;
7-[(dimethylamino)methyl]-14-[(1R,2S) or (1S,2R)-2-fluorocyclohexyl]-3-(1-pyridazin-3-ylethoxy)-7,8-dihydro-6H-indolo[1,2-e][1,5]benzoxazocine-11-carboxylic acid;
7-[(dimethylamino)methyl]-14-[(1R,2S) or (1S,2R)-2-fluorocyclohexyl]-3-(pyridin-2-yloxy)-7,8-dihydro-6H-indolo[1,2-e][1,5]benzoxazocine-11-carboxylic acid;
7-[(dimethylamino)methyl]-14-[(1R,2S) or (1S,2R)-2-fluorocyclohexyl]-3-methoxy-7,8-dihydro-6H-indolo[1,2-e][1,5]benzoxazocine-11-carboxylic acid;
7-[2-(dimethylamino)ethyl]-14-[(1R,2S) or (1S,2R)-2-fluorocyclohexyl]-3-(pyridin-2-ylmethoxy)-7,8-dihydro-6H-indolo[1,2-e][1,5]benzoxazocine-11-carboxylic acid;
7-[2-(dimethylamino)ethyl]-14-[(1R,2S) or (1S,2R)-2-fluorocyclohexyl]-3-(pyridazin-3-ylmethoxy)-7,8-dihydro-6H-indolo[1,2-e][1,5]benzoxazocine-11-carboxylic acid;
7-[2-(dimethylamino)ethyl]-14-[(1R,2S) or (1S,2R)-2-fluorocyclohexyl]-3-(1-pyridin-3-ylethoxy)-7,8-dihydro-6H-indolo[1,2-e][1,5]benzoxazocine-11-carboxylic acid;
7-[2-(dimethylamino)ethyl]-14-[(1R,2S) or (1S,2R)-2-fluorocyclohexyl]-3-(1-pyridin-2-ylethoxy)-7,8-dihydro-6H-indolo[1,2-e][1,5]benzoxazocine-11-carboxylic acid;
7-[2-(dimethylamino)ethyl]-14-[(1R,2S) or (1S,2R)-2-fluorocyclohexyl]-3-(1-pyridazin-3-ylethoxy)-7,8-dihydro-6H-indolo[1,2-e][1,5]benzoxazocine-11-carboxylic acid;
7-[2-(dimethylamino)ethyl]-14-[(1R,2S) or (1S,2R)-2-fluorocyclohexyl]-3-(pyridin-2-yloxy)-7,8-dihydro-6H-indolo[1,2-e][1,5]benzoxazocine-11-carboxylic acid;
7-[2-(diethylamino)ethyl]-14-[(1R,2S) or (1S,2R)-2-fluorocyclohexyl]-3-(pyridin-3-ylmethoxy)-7,8-dihydro-6H-indolo[1,2-e][1,5]benzoxazocine-11-carboxylic acid;
7-[2-(diethylamino)ethyl]-14-[(1R,2S) or (1S,2R)-2-fluorocyclohexyl]-3-(pyridin-2-ylmethoxy)-7,8-dihydro-6H-indolo[1,2-e][1,5]benzoxazocine-11-carboxylic acid;
7-[2-(diethylamino)ethyl]-14-[(1R,2S) or (1S,2R)-2-fluorocyclohexyl]-3-(pyridazin-3-ylmethoxy)-7,8-dihydro-6H-indolo[1,2-e][1,5]benzoxazocine-11-carboxylic acid;
7-[2-(diethylamino)ethyl]-14-[(1R,2S) or (1S,2R)-2-fluorocyclohexyl]-3-(1-pyridin-3-ylethoxy)-7,8-dihydro-6H-indolo[1,2-e][1,5]benzoxazocine-11-carboxylic acid;
7-[2-(diethylamino)ethyl]-14-[(1R,2S) or (1S,2R)-2-fluorocyclohexyl]-3-(1-pyridin-2-ylethoxy)-7,8-dihydro-6H-indolo[1,2-e][1,5]benzoxazocine-11-carboxylic acid;
7-[2-(diethylamino)ethyl]-14-[(1R,2S) or (1S,2R)-fluorocyclohexyl]-3-(1-pyridazin-5-ylethoxy)-7,8-dihydro-6H-indolo[1,2-e][1,5]benzoxazocine-11-carboxylic acid;
7-[2-(diethylamino)ethyl]-14-[(1R,2S) or (1S,2R)-2-fluorocyclohexyl]-3-(pyridin-2-yloxy)-7,8-dihydro-6H-indolo[1,2-e][1,5]benzoxazocine-11-carboxylic acid;
7-[2-(diethylamino)ethyl]-14-[(1R,2S) or (1S,2R)-2-fluorocyclohexyl]-3-methoxy-7,8-dihydro-6H-indolo[1,2-e][1,5]benzoxazocine-11-carboxylic acid;
3-(benzyloxy)-7-{[[2<dimethylamino)ethyl](methyl)amino]methyl}-14-[(1R,2S) or (1S,2R)-2-fluorocyclohexyl]-7,8-dihydro-6H-indolo[1,2-e][1,5]benzoxazocine-11-carboxylic acid;
7-{[[2-(dimethylamino)ethyl](methyl)amino]methyl}-14-[(1R,2S) or (1S,2R)-2-fluorocyclohexyl]-3-(1-phenylethoxy)-7,8-dihydro-6H-indolo[1,2-e][1,5]benzoxazocine-11-carboxylic acid;
3-(benzyloxy)-7-[(dimethylamino)methyl]-14-[(1R,2S) or (1S,2R)-2-fluorocyclohexyl]-7,8-dihydro-6H-indolo[1,2-e][1,5]benzoxazocine-11-carboxylic acid;
7-[(dimethylamino)methyl]-14-[(1R,2S) or (1S,2R)-2-fluorocyclohexyl]-3-(1-phenylethoxy)-7,8-dihydro-6H-indolo[1,2-e][1,5]benzoxazocine-11-carboxylic acid;
3-(benzyloxy)-7-[2-(dimethylamino)ethyl]-14-[(1R,2S) or (1S,2R)-2-fluorocyclohexyl]-7,8-dihydro-6H-indolo[1,2-e][1,5]benzoxazocine-11-carboxylic acid;
7-[2-(dimethylamino)ethyl]-14-[(1R,2S) or (1S,2R)-2-fluorocyclohexyl]-3-(1-phenylethoxy)-7,8-dihydro-6H-indolo[1,2-e][1,5]benzoxazocine-11-carboxylic acid;
3-(benzyloxy)-7-[2-(diethylamino)ethyl]-14-[(1R,2S) or (1S,2R)-2-fluorocyclohexyl]-7,8-dihydro-6H-indolo[1,2-e][1,5]benzoxazocine-11-carboxylic acid;
7-[2-(diethylamino)ethyl]-14-[(1R,2S) or (1S,2R)-2-fluorocyclohexyl]-3-(1-phenylethoxy)-7,8-dihydro-6H-indolo[1,2-e][1,5]benzoxazocine-11-carboxylic acid;
or a pharmaceutically acceptable salts salt thereof.

16. (canceled)

17. (canceled)

18. A pharmaceutical composition comprising a compound as claimed in claim 1, or a pharmaceutically acceptable salt thereof, in association with a pharmaceutically acceptable carrier.

19. The pharmaceutical composition as claimed in claim 18 further comprising one or more other agents for the treatment of viral infections such as an antiviral agent, or an immunomodulatory agent such as α-, β- or γ-interferon.

20. A method of inhibiting hepatitis C virus polymerase and/or of treating or preventing an illness due to hepatitis C virus, the method involving administering to a human or animal subject suffering from the condition a therapeutically or prophylactically effective amount of a pharmaceutical composition as claimed in claim 18.

21. (canceled)

22. A process for the preparation of a compound as claimed in claim 1:
(a) where Y is NR¹⁴, by internal ring closure of a compound of formula (II): 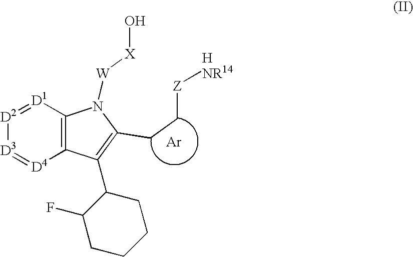
wherein R¹⁴, D¹, D², D³, D⁴, Ar, W, X and Z are defined in claim 1;
(b) where Y is NR¹⁴ and Z is —CH₂—, by reduction and internal ring closure of a compound of formula (III): 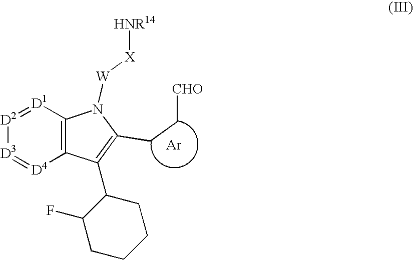
wherein R¹⁴, D¹, D², D³, D⁴, Ar, W and X are as defined in claim 1; or
(c) by internal ring closure of a compound of formula (IV): 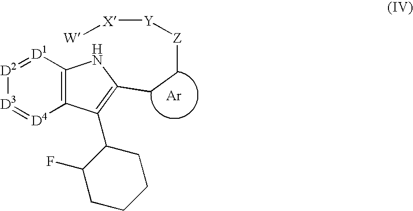
wherein R¹, R², A, Ar, Y and Z are as defined in claim 1, and X′ is X as defined in claim 1 or is converted to X during or after the cyclisation reaction, and W′ is W as defined in claim 1 or is converted to W during or after the cyclisation reaction.
